(19) United States
(12) Patent Application Publication
(10) Pub. No.: US 2022/0145264 A1
(43) Pub. Date: May 12, 2022

(54) CULTURE MEDIUM FOR MAMMALIAN EXPANDED POTENTIAL STEM CELLS, COMPOSITION, AND METHODS THEREOF

(71) Applicant: THE UNIVERSITY OF HONG KONG, Hong Kong (CN)

(72) Inventors: Pengtao LIU, Hong Kong (CN); Xuefei GAO, Hong Kong (CN); Degong RUAN, Hong Kong (CN)

(21) Appl. No.: 17/435,943

(22) PCT Filed: Mar. 27, 2020
(86) PCT No.: PCT/CN2020/081594

§ 371 Date: Sep. 2, 2021

Related U.S. Application Data
(60) Provisional application No. 62/829,904, filed on Apr. 5, 2019.

(51) Int. Cl. C12N 5/074; C12N 5/0735

(52) U.S. Cl. CPC C12N 5/0696; C12N 5/0606; C12N 2506/1307; C12N 2501/235; C12N 2500/36; C12N 2500/44; C12N 2500/38; C12N 2500/46; C12N 2501/16

(57) ABSTRACT

A culture medium is provided for establishing expanded potential stem cell (EPSC) lines for mammals. Methods are provided using the medium for the in vitro conversion and maintenance of cells, including pluripotent cells into EPSCs.

### 1. FIELD

A culture medium is provided for establishing expanded potential stem cell (EPSC) lines for mammals. Methods are provided using the medium for the in vitro conversion and maintenance of cells, including pluripotent cells into EPSCs.

### 2. BACKGROUND

Mammalian embryonic development begins when a sperm and an egg fuse to form a zygote, which undergoes a fixed number of divisions. Up to the 8 cells (8C) stage, an embryo has the capacity to differentiate to all lineages in the embryo proper and extraembryonic tissues and are considered totipotent (Ishiuchi et al 2013). Subsequent cell divisions produce two of the earliest lineages: the trophectoderm epithelium (TE) cells which are restricted to the trophoblast lineage and are essential for the formation of the placenta, and the inner cell mass (ICM) which are pluripotent and give rise to all cell types of the embryo proper, as well as to extra-embryonic endoderm and mesoderm, and embryonic stem (ES) cells (Gardner 1985, Rossant et al 2009, Yamanaka et al 2006).

Although ES cells are capable of differentiating into all germ cell layers of the embryo when returned to the blastocyst environment, they are generally unable to contribute to the trophoblast lineage. Conversely, trophoblast stem cells, which are derived from the trophectoderm can efficiently differentiate into trophoblasts in vitro and in vivo. However, they are unable to differentiate into all germ cell layers of the embryo.

Human embryonic stem cells have been reported to differentiate to trophoblasts in vitro under certain conditions, but there is debate as to whether these in vitro differentiated trophoblasts are bona fide trophoblasts (see, Roberts R M et al 2014) When cultured in vitro, human embryonic stem cells show distinct molecular and biological characteristics that are different from the paradigmatic embryonic stem cells. The terminology ‘naïve’ (or ‘ground state’) and ‘primed’ was introduced to describe the observed differences.

Recently, several researchers have reported alternative conditions for inducing a more ‘naïve’ pluripotent state in conventional human embryonic stem cells, for example, by culturing in a mix of inhibitors (summarised in Theunissen et al 2014). However, although cells produced by these methods display some characteristics which are comparable to naive cells, there are also significant differences.

Despite these findings, it remains unclear whether it is possible to experimentally generate and maintain bona fide pluripotent stem cells from important mammalian animal species, in particular large farm animals. The need remains for improved human pluripotent stem cells for studying human development, biology, and regenerative medicine remains.

### 3. SUMMARY

Provided herein is a culture medium for establishing expanded potential stem cell (EPSC) lines which resemble naïve or ground state embryonic stem cells, but are also able to differentiate into placenta trophoblasts and the embryo proper.

In one embodiment of the present disclosure is a porcine stem cell culture medium, comprising a basal medium comprising SRC inhibitor, Vitamin C supplement, LIF protein, and ACTIVIN protein. In certain embodiments, the basal medium is DMEM/F-12. In certain embodiments, the basal medium is DMEM. In certain embodiments, the SRC inhibitor is WH-4-023 and XAV939. In certain embodiments, the medium further comprises N2 supplement, B27 supplement, Glutamine Penicillin-Streptomycin, NEAA, 2-mercaptoethanol, CHIR99021, and FBS.

In one embodiment of the present disclosure is a porcine stem cell culture medium, comprising a basal medium comprising SRC inhibitor, Vitamin C supplement, and LIF protein. In certain embodiments, the basal medium is DMEM/F-12. In certain embodiments, the basal medium is DMEM. In certain embodiments, the SRC inhibitor is A-419259 and XAV939. In certain embodiments, the medium further comprises N2 supplement, B27 supplement, Glutamine Penicillin-Streptomycin, NEAA, 2-mercaptoethanol, and CHIR99021.

In one embodiment of the present disclosure is a porcine stem cell culture medium, comprising a basal medium comprising ITS-X 200, Vitamin C supplement, Bovine Albumin Fraction V, Trace elements B, Trace elements C, Reduced glutathione, Defined lipids, SRC inhibitor, endo-IWR-1, SRK inhibitor, and Chiron 99021. In certain embodiments, the basal medium is DMEM/F-12. In certain embodiments, the basal medium is DMEM. In certain embodiments, the SRC inhibitor is XAV939. In certain embodiments, the SRK inhibitor is A-419259. In certain embodiments, the medium further comprises Neurobasal medium, Penicillin-Streptomycin-Glutamine, NEAA, Sodium Pyruvate, 2-Mercaptoethanol, N2, B27, Human Lif protein.

In one embodiment of the present disclosure is a porcine stem cell culture medium, comprising a basal medium comprising ITS-X 200, Vitamin C supplement, Bovine Albumin Fraction V, Trace elements B, Trace elements C, Reduced glutathione, SRC inhibitor, endo-IWR-1, Chiron 99021, Human Lif protein, and Activin A. In certain embodiments, the basal medium is DMEM/F-12. In certain embodiments, the basal medium is DMEM. In certain embodiments, the SRC inhibitor is WH-4-023 and XAV939. In certain embodiments, the medium further comprises Neurobasal medium, Penicillin-Streptomycin-Glutamine, NEAA, Sodium Pyruvate, 2-Mercaptoethanol, N2, and B27.

One embodiment of the present disclosure is a method for producing a population of porcine expanded potential stem cells (EPSCs) which comprises: (i) Providing a population of pluripotent cells, and (ii) Culturing the population in the stem cell disclosed herein.

### 4. Brief Description of the Drawings

FIG. 1. Derivation and characterization of porcine EPSCs. a. Left: Schematic diagram of establishment of the porcine (Sus Scrofa) EPSC^{Emb} lines from German Landrace day-5 in vivo derived blastocysts on STO feeder cells in pEPSCM, and of pEPSC^{iPS} lines by reprogramming German Landrace PFFs and China TAIHU OCT4-Tdtomato knock-in reporter (POT) PFFs. Right panels: images of established EPSC lines, and a fluorescence image of Td-tomato expression in POT-pEPSC^{iPS}. Three EPSC^{Emb} lines (Male: K3 and K5; Female K1) and three pEPSC^{iPS} lines (#10, #11) were extensively tested in this study. These EPSC lines behaved similarly in gene expression and differentiation. b. Bisulphite sequencing analysis of CpG sites in the OCT4 and NANOG promoter regions in PFFs, pEPSC^{iPS} and pEPSC^{Emb}. c. Gene expression in embryoid bodies (EBs, day 7) of pEPSCs^{Emb}. Genes of both embryonic and extra-embryonic cell lineages were examined in RT-qPCR. Relative expression levels are shown with normalization to GAPDH. Data are mean±s.d. (n=3). *p<0.01 compared to pEPSCs^{Emb}. d. Tissue composition of pEPSC^{Emb} teratoma sections (H&E staining): Examples of glandular epithelium derived from endoderm (i), cartilage derived from mesoderm (ii), immature neural tissue derived from ectoderm, which forms well defined neural tubes (iii), and large multinucleated cells reminiscent of trophoblasts (arrows in iv). e. PL-1 and KRT7 positive cells in pEPSC^{Emb} teratoma sections as revealed by immunostaining. f. Schematic diagram of day 25-27 porcine chimeric conceptuses. The circles mark the areas where cryo-sections for immunofluorescence staining in g. were taken: i, central nervous system; ii, fetal liver. g. Detection of pEPSC descendants in the brain (H2BmCherry⁺SOX2⁺) and the liver (H2BmCherry⁺AFP⁺) in chimera ^{#}16. H2B-mCherry and SOX2 are nuclear localised whereas AFP is a cytoplasmic protein. Boxed areas are shown in higher magnification. Arrows indicate representative cells that are donor cell descendants (mCherry⁺). DAPI stains nuclei. Additional chimera analyses are presented in Extended Data FIG. 5e-5f.

FIG. 2. In vitro generation of PGC-like cells from pEPSCs^{Emb}. a. Induction of pPGCLC by transiently expressing SOX17 in NANOS3-H2BmCherry reporter pEPSCs. The presence of H2BmCherry⁺TNAP⁺ cells in embryoid bodies (EBs) was analysed by FACS. b. RT-qPCR analysis of PGC genes in day 3 EBs following pPGCLC induction. Relative expression levels are shown with normalization to GAPDH. Data are mean±s.d. (n=3). * p<0.01 compared with non-transfected EBs. c. Immunofluorescence analysis of PGC factors in the sections of day 3-4 EBs of pPGCLC induction. The H2BmCherry⁺ cells co-expressed NANOG, OCT4, BLIMPL TFAP2C and SOX17. DAPI stains nuclei. Experiments were performed at least three times. d. RNAseq analysis (Heat map) of sorted H2BmCherry⁺ of pPGCLC induction shows expression of genes associated with PGCs, pluripotency or somatic lineages (mesoderm, endoderm, and gonadal somatic cells). e. Pair-wise gene expression comparison between pEPSCs^{Emb} and pPGCLCs. Key up-regulated (red) and down-regulated (blue) genes are highlighted. f. Bar plot shows expression of genes related to DNA methylation in pPGCLCs and the parental pEPSCs^{Emb}. Data are from RNAseq of sorted H2BmCherry⁺ of pPGCLC induction. Each sample has two biological replicates, and the bar plot displays the average expression of the two replicates.

FIG. 3. Establishment of human EPSCs. a. Images of the established H1-EPSCs or M1-EPSCs (passage 25). b. Principal component analysis (PCA) of bulk RNA-seq gene expression data of human, porcine and mouse EPSCs, human primed and naïve ESCs, PFFs. pEPSC^{Par}: EPSC lines from parthenogenetic embryos; E14 and AB2-EPSCs are mouse EPSCs. c. Pair-wise comparison of gene expression between H1-ESCs and H1-EPSCs, showing the highly expressed genes (>8 folds) in hEPSCs (total 76, red dots) and representative histone genes (blue dots). d. Heatmap showing expression of selected histone genes in H1-ESCs, H1-EPSCs, iPSC-EPSCs and human naïve (5i) ESCs, and human preimplantation embryos. RNAseq data of human primed and naïve ESCs were obtained from ref 42, whereas embryo cell data were from ref 44. e. RT-qPCR analysis of expression of four histone 1 cluster genes in seven human ESC or iPSC lines cultured in the three conditions: FGF (primed), 5i (naïve) and EPSCM (EPSC). Hipsci iPSC lines were obtained from the Hipsc project at the Wellcome Trust Sanger Institute (http://www.hipsci.org): ^{#}1, HPSI1113i-bima_1; ^{#}2, HPSI1113i-qolg_3; ^{#}3, HPSI1113i-oaaz_2; ^{#}4, HPSI1113i-uofv_1. Relative expression levels are shown with normalization to GAPDH. Data are mean±s.d. (n=3). * p<0.01 compared with the FGF condition cultured cells. ^{#}p<0.01 compared with 5i condition cultured cells. Experiments were performed at least three times. f. Violin plots show scRNAseq expression of pluripotency genes in pEPSCs^{Emb} (top panel) and human H1-EPSCs (lower panel). g. PCA of global gene expression pattern (by scRNAseq) of pEPSCs^{Emb} (left panel) and H1-EPSCs (right panel). h. PCA and comparison of gene expression from scRNAseq of human H1-EPSCs and human preimplantation embryos (ref 46. See Methods for details). i. ChIP-seq analysis of H3K27me3 and H3K4me3 marks at pluripotency gene loci in pEPSCs^{Emb} and human H1-EPSCs.

FIG. 4. Trophoblast differentiation potential of human EPSCs. a. Left panel: diagram of hEPSCs to trophoblast under TGFβ inhibition. See Methods for more details. Right panel: flow cytometry analysis of differentiation of the CDX2-H2B-Venus reporter EPSCs to trophoblasts. The CDX2-H2B-Venus reporter EPSCs were also cultured in conventional FGF-containing hESCs medium or 5i-naïve medium and were subsequently subjected to the same differentiation conditions and examined in flow cytometry. Cells were collected 4 days after TGFβ inhibition. b. The dynamic changes in the expression of trophoblast genes during hEPSC differentiation at several time points were assayed by RT-qPCR. Relative expression levels are shown with normalization to GAPDH. Data are mean±s.d. (n=3). *p<0.01 compared with H1-ESC cells. ^{#}p<0.01 compared with H1-5i cells. Experiments were performed at least three times. c. tSNE analysis of RNA-seq data of the differentiated cells from H1-ESCs, H1-EPSCs, or iPSC-EPSCs treated with the TGFβ inhibitor SB431542. RNAs were sampled at Day 0-12 during differentiation. The differentiation trajectory of H1-EPSCs and hiPSC-EPSCs is distinct from that of H1-ESCs. d. Phase-contrast images of primary TSC colonies formed from individual hEPSCs (left) and of TSCs at passage 7 (right). e. Expression of trophoblast transcription factors GATA3 and TFAP2C, and KRT7 in EPSC-TSCs detected by immunostaining. Nuclei were stained with DAPI. Similar results were obtained with four independent EPSC-TSC lines. f. Expression of SDC1 in syncytiotrophoblasts differentiated from EPSC-TSCs as detected by immunofluorescence. DAPI stains the nucleus. g. Flow cytometry detection of HLA-G in hESCs, hEPSCs, hTSCs generated in this study, and cells differentiated from hTSCs following the EVT protocol (ref 53). The choriocarcinoma cells JEG-3, which are representatives for extravillous trophoblasts, express HLA-G, and JAR that are representative for villous trophoblast cells so do not express any HLA molecules (Apps, R., et al. Immunology 2009), were used as the positive and negative control, respectively. h. Confocal images of immunostaining for SDC1- or KRT7-positive cells in lesions formed from injected hTSCs in immunocompromised mice. DAPI stains the nucleus. Experiments were performed at least three times.

### 4.1 Extended Data Figures

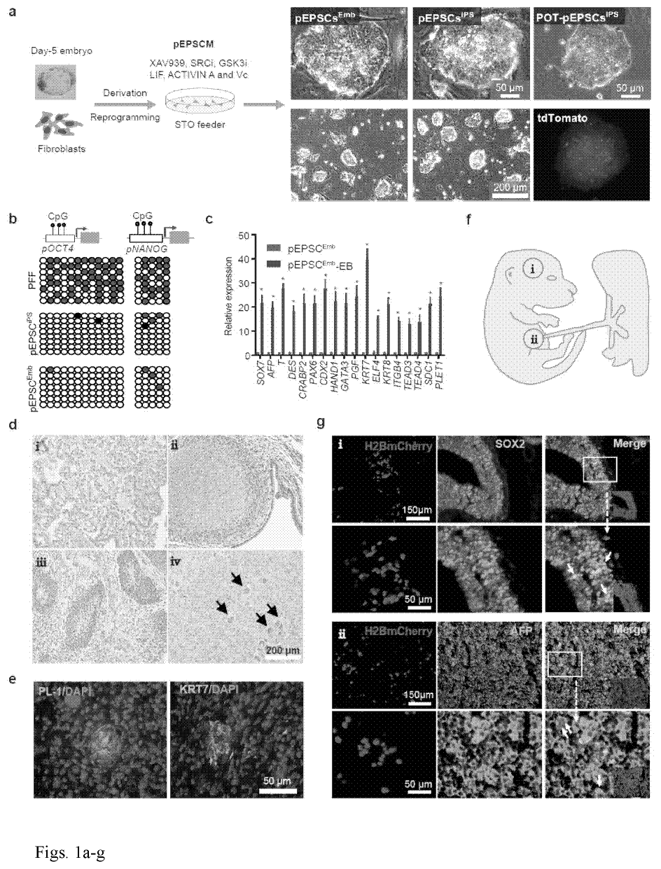

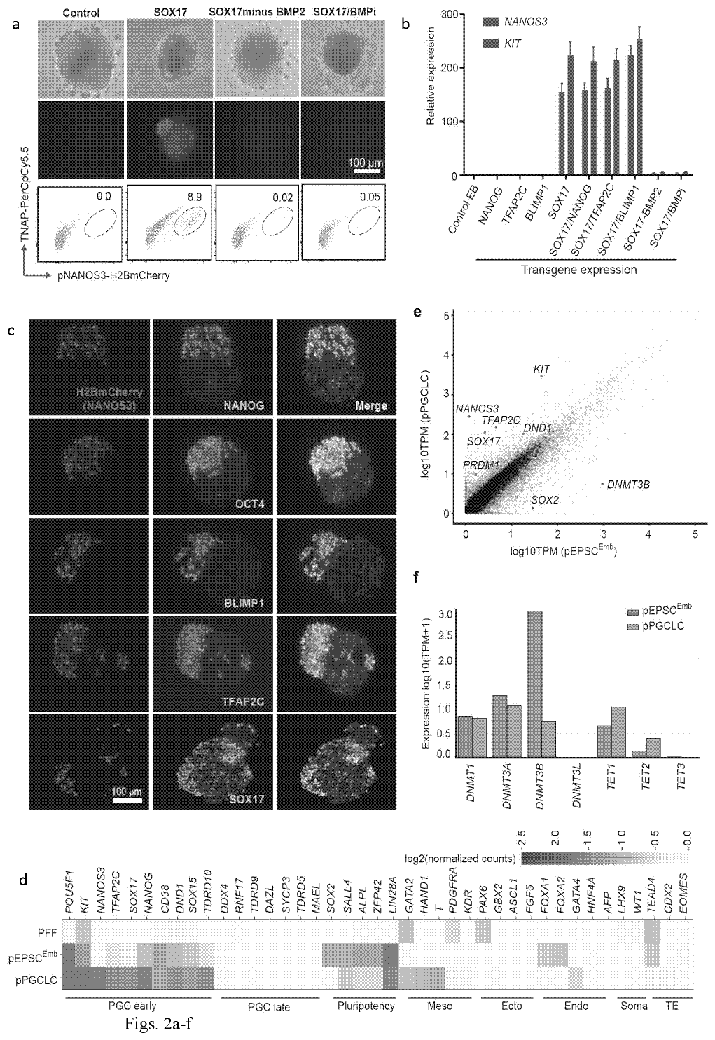

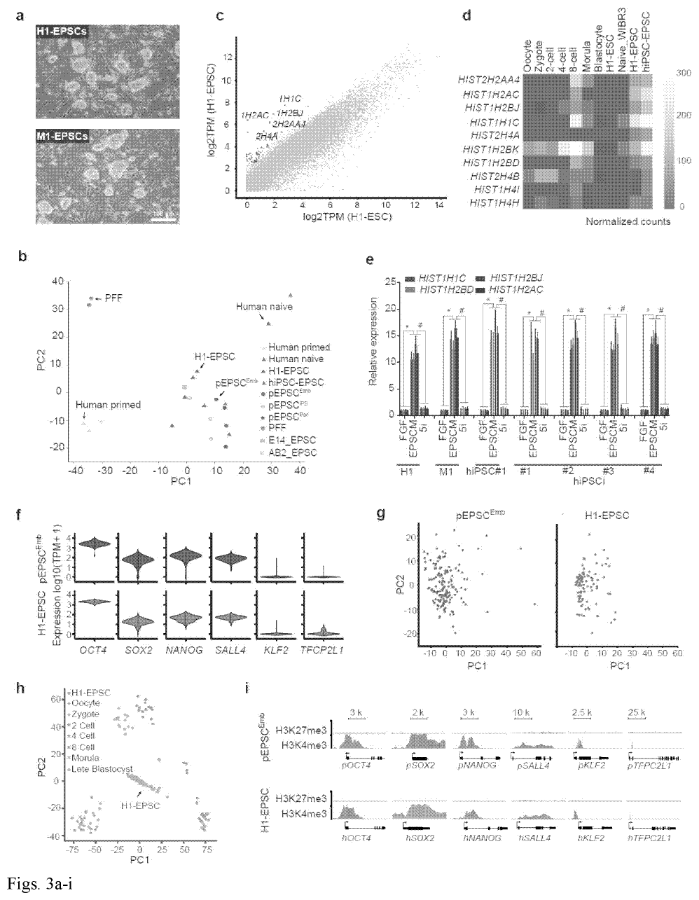

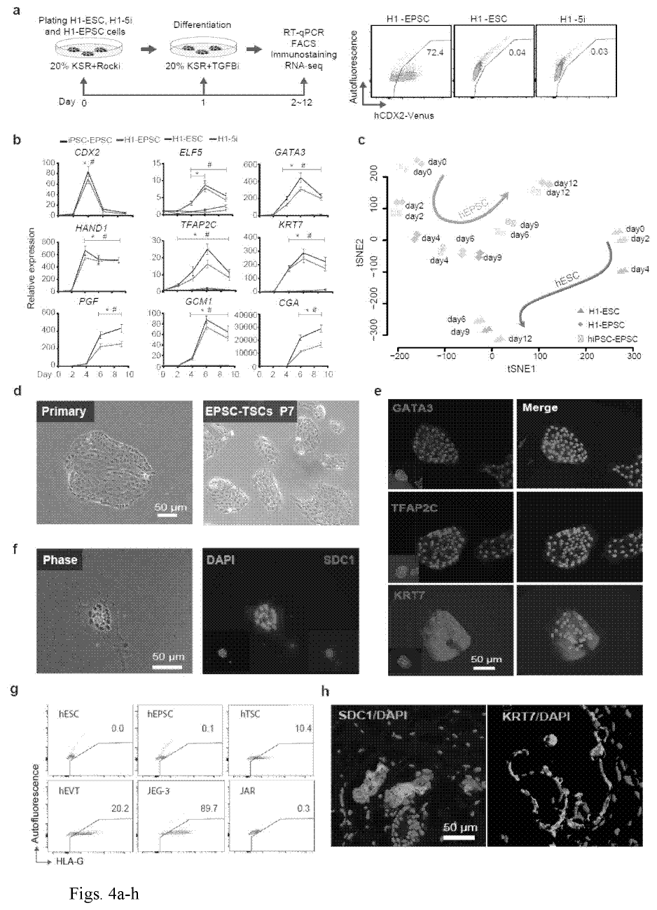

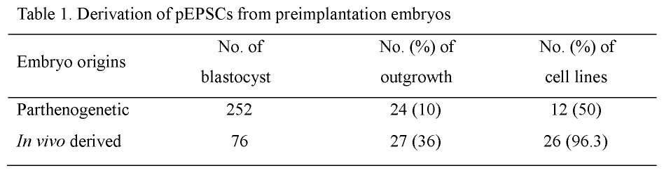

Extended Data FIG. 1. Establishment of new Dox-dependent porcine iPSC lines for screening culture conditions. a. Doxycycline (Dox)-inducible expression of Yamanaka factors OCT4, MYC, SOX2 and KLF4, together with LIN28, NANOG, LRH1 and RARG in wild type German Landrace PFFs. cDNAs were cloned into piggyBac (PB) vectors and transfected into PFFs with a plasmid expressing the PB transposase for stable integration of the expression cassette into the porcine genome. pOMSK: Porcine origin 4 Yamanaka factors OCT4, MYC, SOX2 and KLF4; pN+hLIN: porcine NANOG and human LIN28; hRL: human RARG and LR111. After 8-10 days of Dox induction, primary colonies appeared. Those colonies were single-cell passaged in the presence of Dox in M15 (15% fetal calf serum). b. Co-expression of LIN28, NANOG, LR111 and RARG substantially increased the number of reprogrammed colonies. *p<0.01. Data are mean±s.d. (n=4): the 8-factor induced colonies from 250,000 PFFs in comparison to those of using the 4 Yamanaka factors. c. Reprogramming of the porcine OCT4-tdTomato knock-in reporter (POT) TAIHU PFFs to iPSCs. After 8 days of Dox induction, primary colonies appeared, which were tdTomato⁺ under fluorescence microscope. The primary colonies were picked and expanded in the presence of Dox. Shown on the images are passage 3 cells of bright field and fluorescence. d. The iPSCs lines expressed key pluripotency genes in RT-qPCR analysis. The iPSC lines ^{#}1 and ^{#}2, and iPSC ^{#}3 and ^{#}4 were from wild type German Landrace and TAIHU POT PFFs, respectively. Gene expression in porcine blastocysts was used as the control. e. RT-qPCR analysis of expression of the exogenous reprogramming factors in iPSCs either in the presence of Dox or 3 days after its removal. f. Differentiation of iPSC cells once Dox had been removed from the culture medium. The images show cells 3 days after Dox removal. The POT iPSCs became Td-tomato negative. g. RT-qPCR analysis of the expression of endogenous pluripotency genes in iPSCs cultured with or without Dox. h. Expression of lineage genes in porcine iPSCs 5-6 days after DOX removal. Gene expression was measured by RT-qPCR. Relative expression levels are shown with normalization to GAPDH. Data are mean±s.d. (n=3). Experiments were performed at least three times.

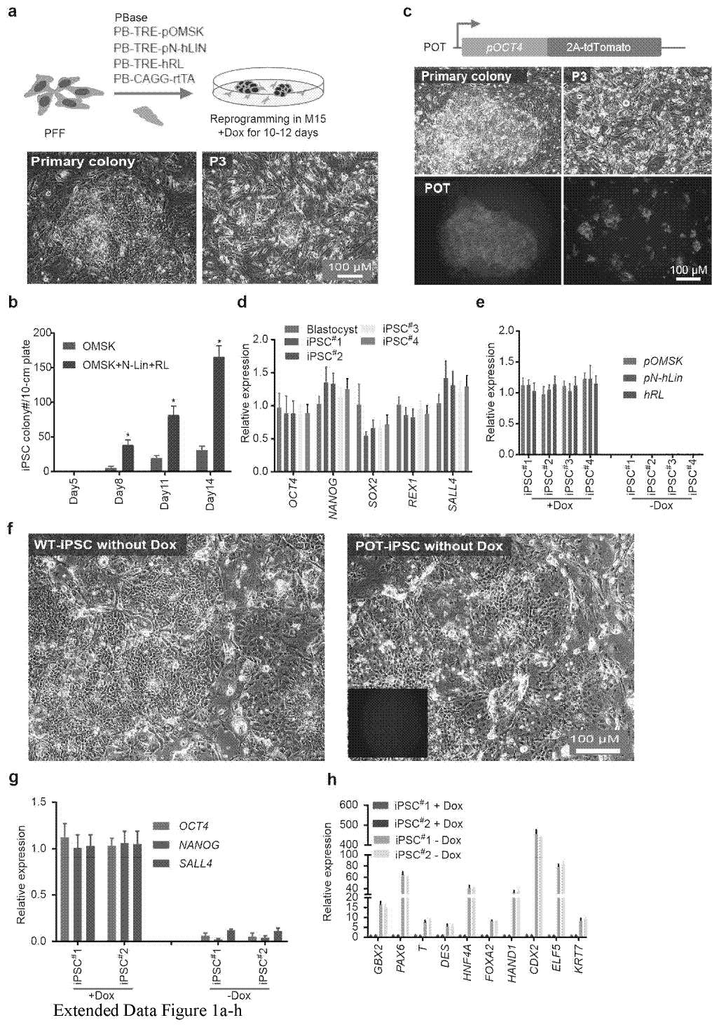

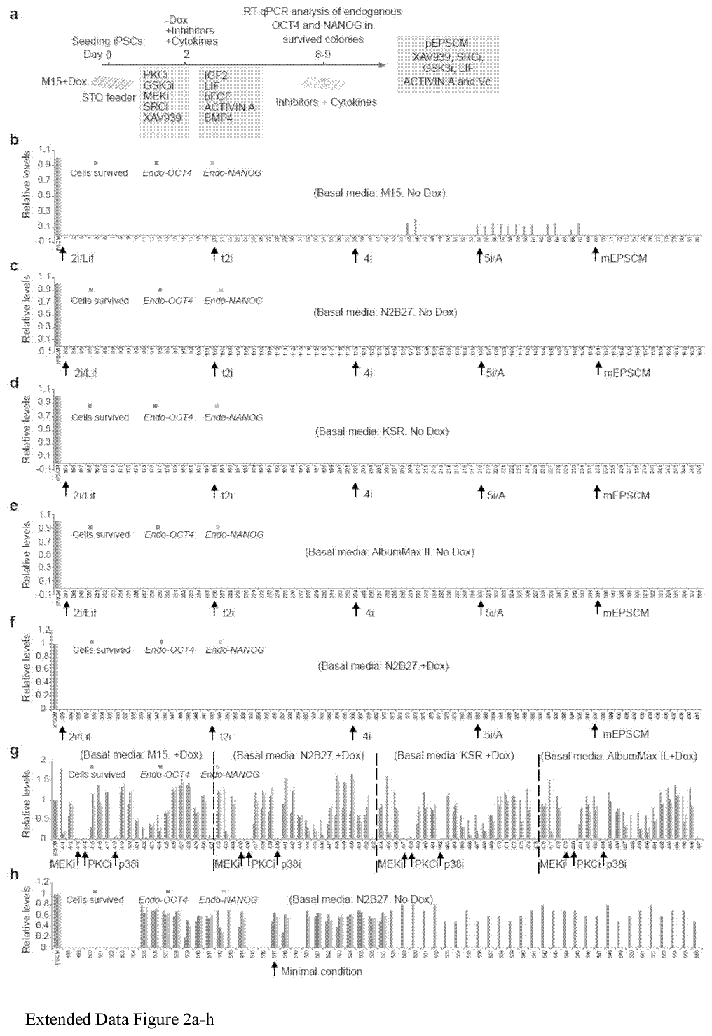

Extended Data FIG. 2. Identification of culture conditions for porcine EPSCs. a. The Dox-dependent iPSC clone ^{#}1 of German Landrace strain was used in the screens. Small molecule inhibitors and cytokines were selected for various combinations. Cell survival, cell morphology, and expression of endogenous OCT4 and NANOG were employed as the read-outs. b-h. The relative expression levels of endogenous OCT4 and NANOG in the survived cells after 6 days of culture in different basal media supplemented with inhibitors and cytokines combinations: b. M15 medium without Dox; c. N2B27 basal medium without Dox; d. 20% KOSR medium without Dox; e. AlbumMax II basal medium without Dox; f. N2B27 basal medium with Dox; g. Four individual basal media with Dox (M15: 411-431; N2B27: 432-453; KOSR: 454-475; AlbumMax II: 476-497); h. N2B27 basal medium without Dox. 2i: GSK3i and MEKi; t2i: GSK3i, MEKi and PKCi (Takashima, Y, et al. 2014 Cell); 4i: GSK3i, MEKi, JNKi and p38i (Irie, N., et al 2015 Cell); 5i: GSK3i, MEKi, ROCKi, BRAFi and SRCi (Theunissen, T. W., et al. 2014 Cell Stem Cell); mEPSCM: GSK3i, MEKi, JNKi, XAV939, SRCi and p38i (Yang J., et al. 2017 Nature); Details of the inhibitor combinations are presented in Supplementary Table 1. Relative expression levels are shown with normalization to GAPDH.

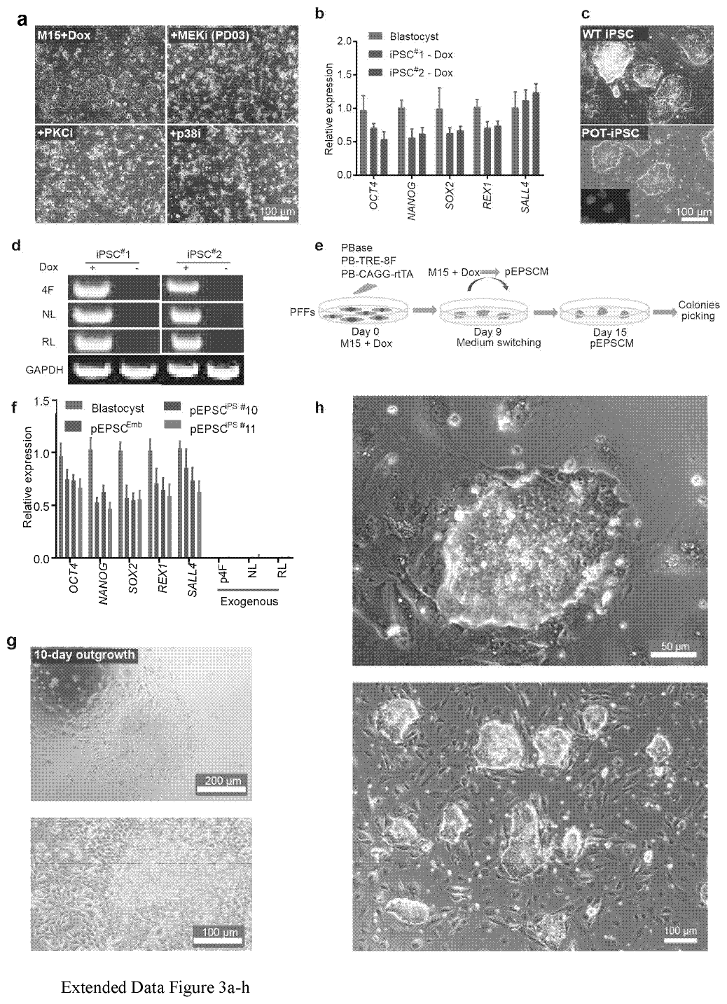

Extended Data FIG. 3. Establishment of porcine EPSCs by reprogramming PFFs or from pre-implantation embryos. a. Images showing the toxicity of MEKi, PKCi and p38i to the porcine iPSCs in M15 plus Dox. b. Endogenous pluripotency gene expression in porcine iPSCs in the absence of Dox in pEPSCM (#517 minimal condition, Extended Data FIG. 2h). Gene expression was compared to that in porcine blastocysts. Data are mean±s.d. (n=3). c. Images of wild type and OCT4-Tdtomato reporter iPSCs in pEPSCM without Dox. Gene expression was compared to that in porcine blastocysts. d. Detection of leaky expression of the exogenous reprogramming factors by RT-PCR. About half of the iPSC lines did not have detectable leaky expression. e. Schematic diagram of reprogramming PFFs to establish EPSC lines in pEPSCM. f. Two newly established WT pEPSCi lines (#10 and #11) were examined for expression of endogenous pluripotency genes and the exogenous reprogramming factors. Data are mean±s.d. (n=3). g. Day-10 outgrowth from a porcine early blastocyst in pEPSCM supplemented with ROCK inhibitor. The outgrowths were picked at day 10-12 for dissociation and re-plating to establish stable lines. h. Representative images of the pEPSC^{Emb} (Line K3) established from porcine in vivo derived embryos. Experiments were performed at least three times. Relative expression levels are shown with normalization to GAPDH.

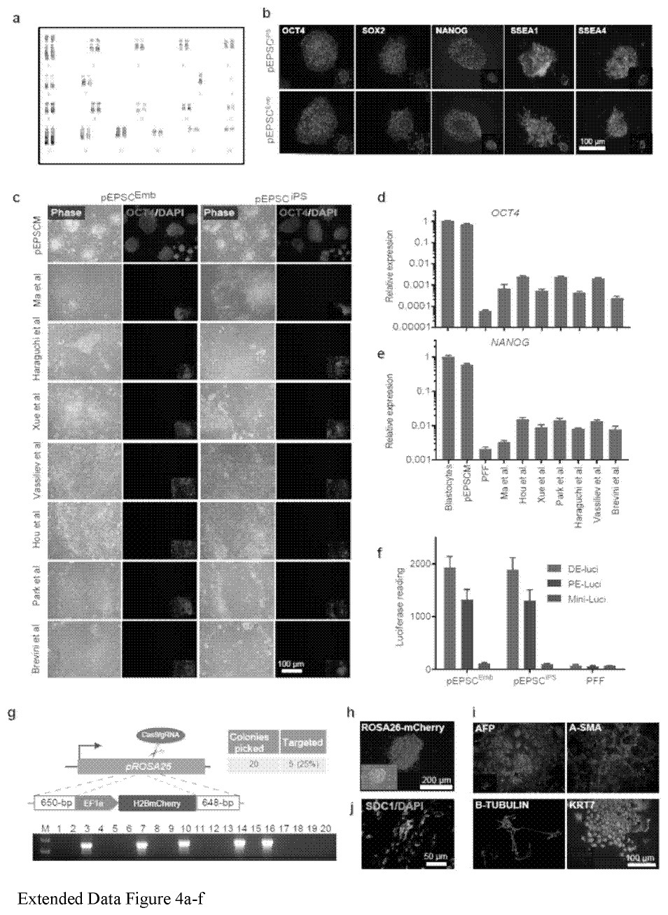

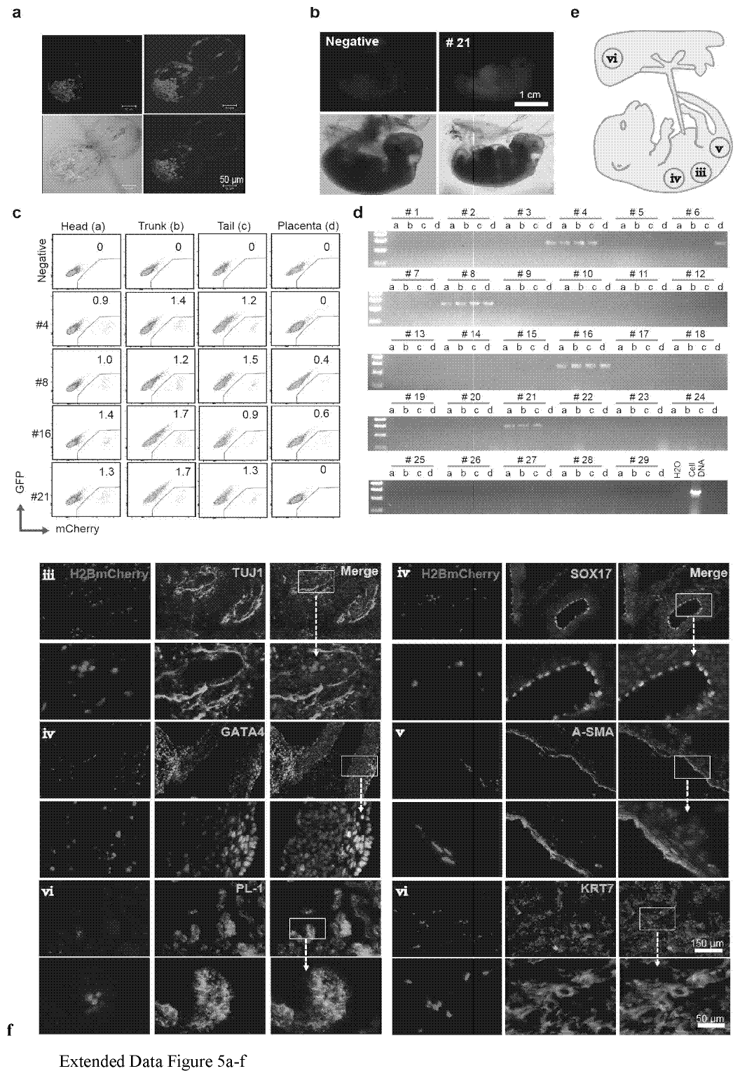

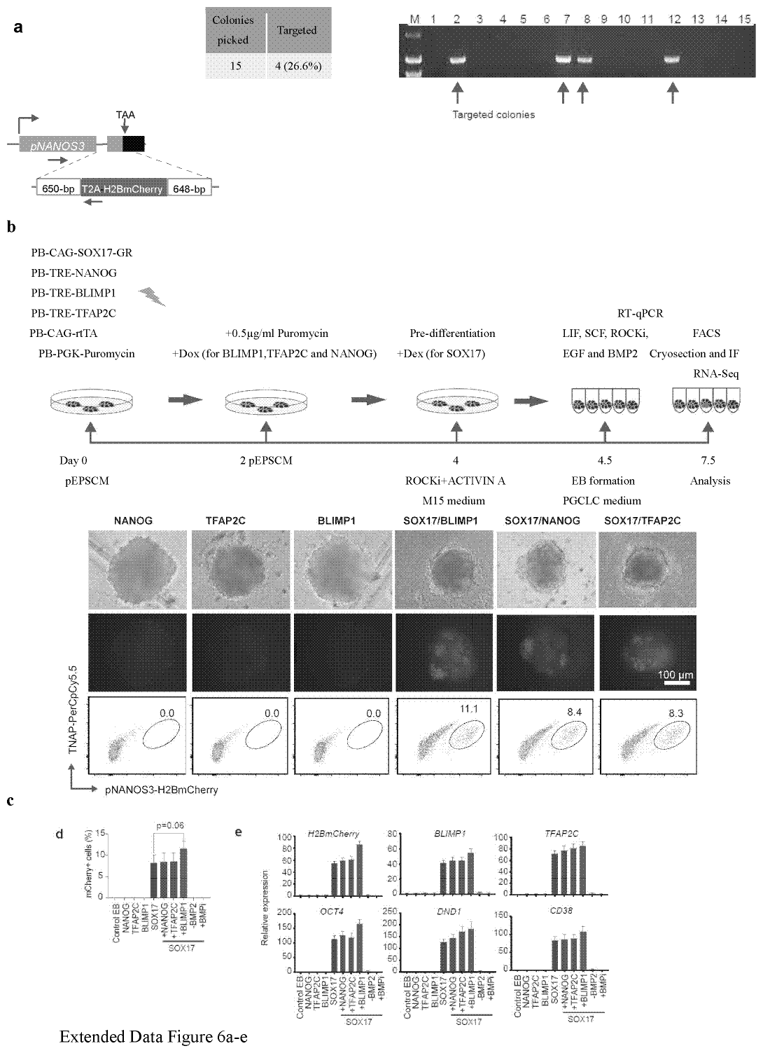

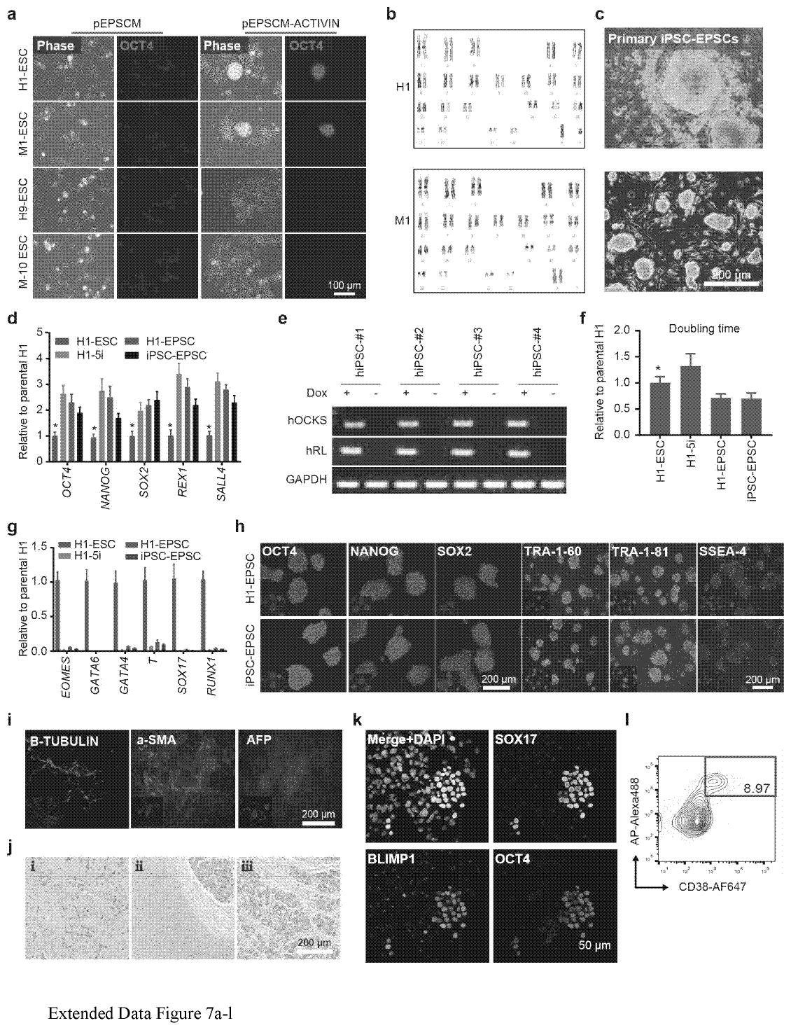

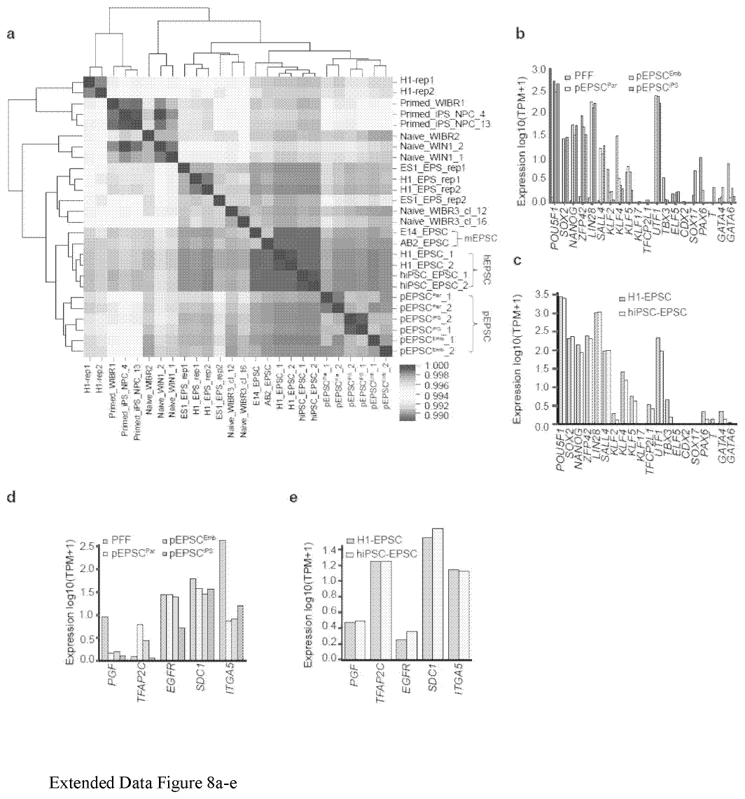

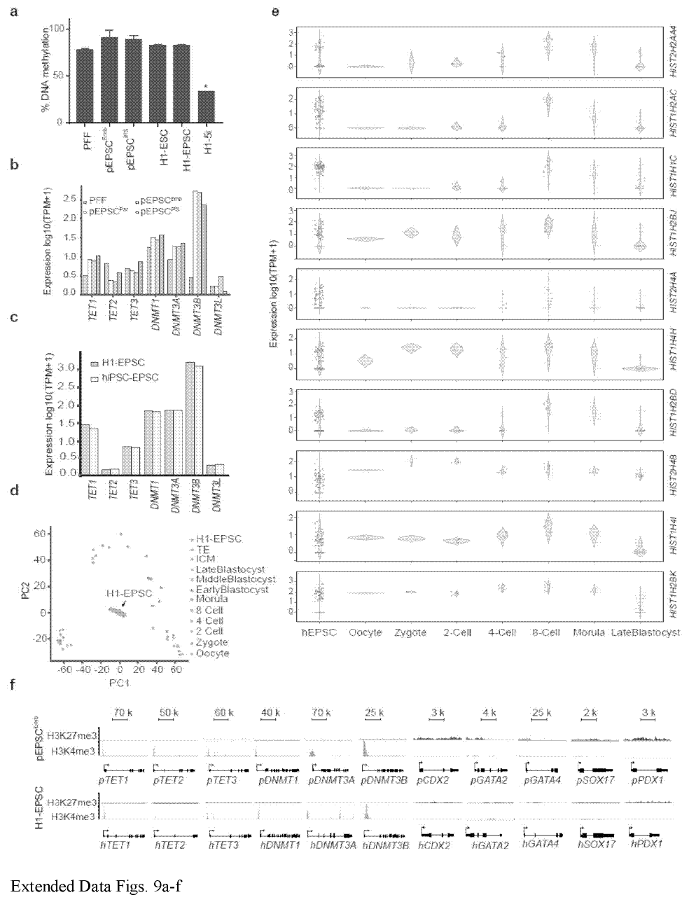

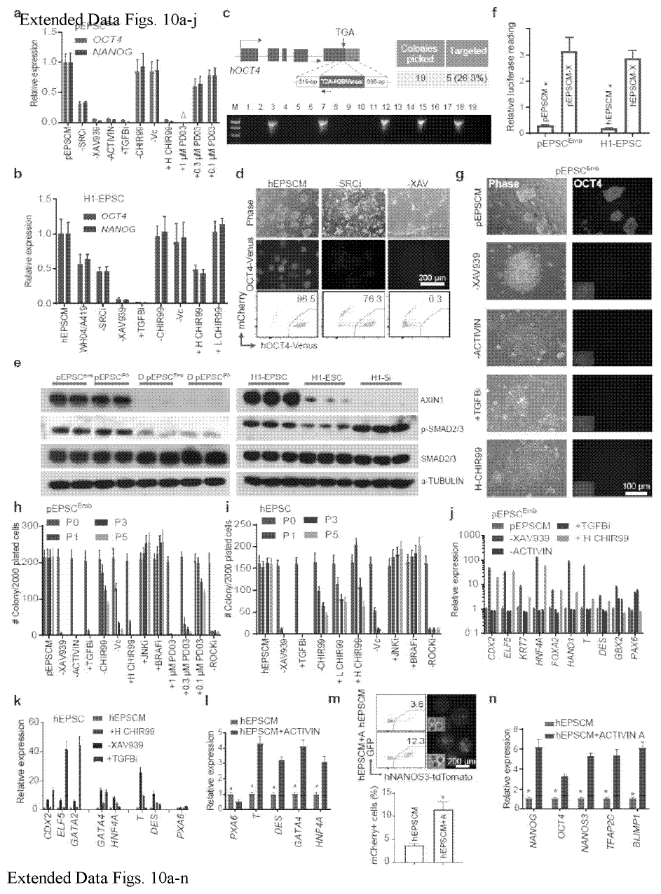

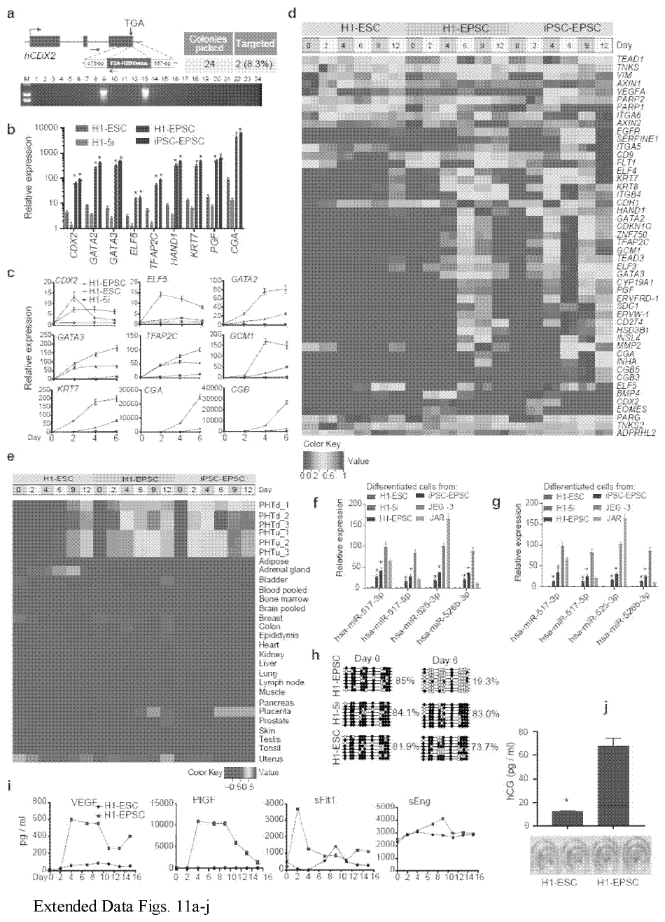

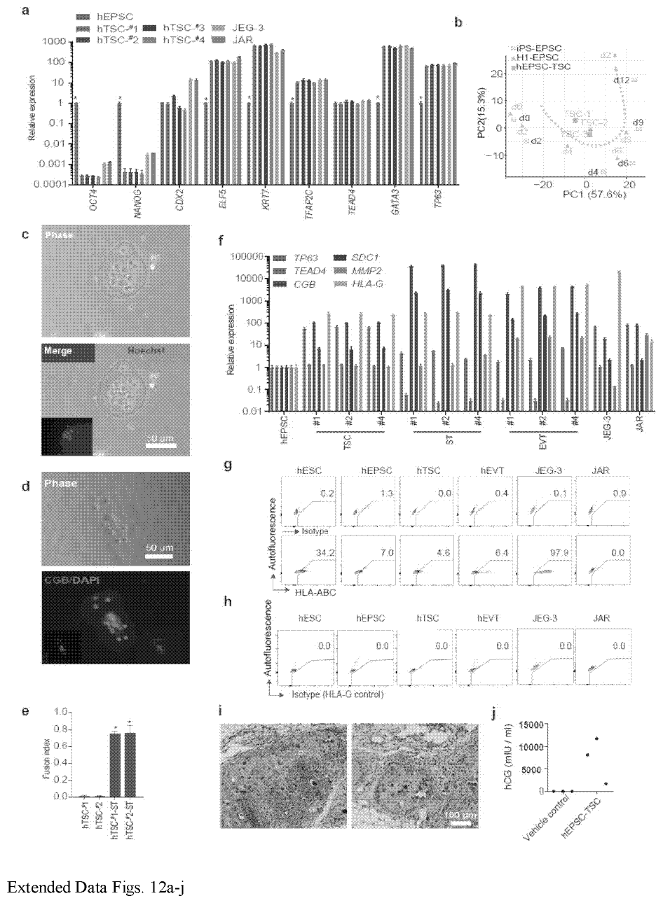

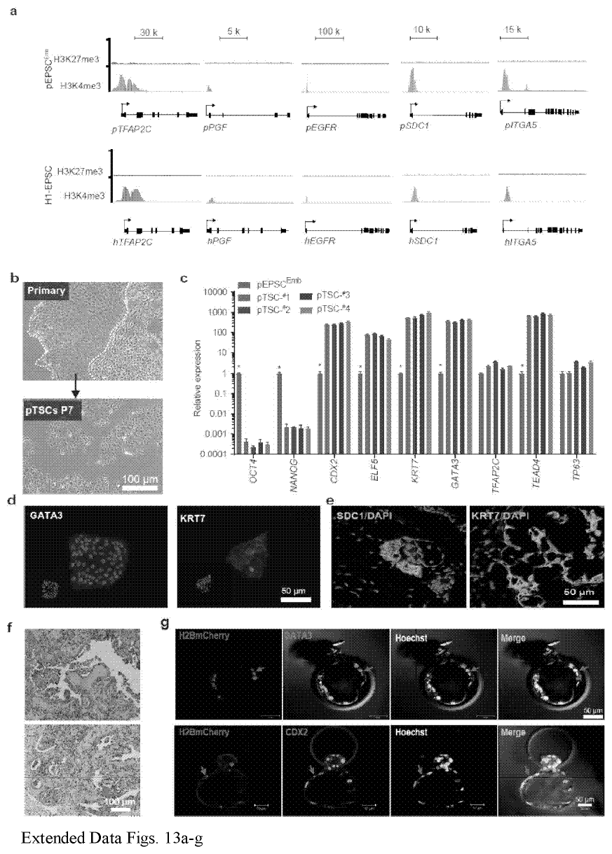

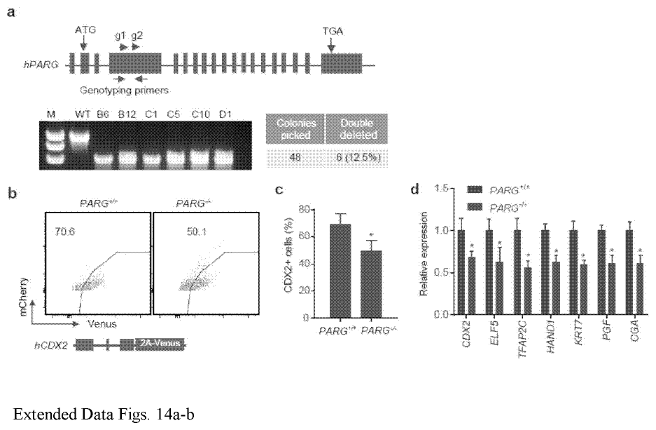

Extended Data FIG. 4. Characterisation of pEPSCs. a. pEPSC^{Emb} (Line K3) retained a normal karyotype after 25 passages (10/10 metaphase spreads examined were normal). Two additional lines examined also had the normal karyotype after more than 25 passages. b. Immunostaining detection of pluripotency factors and markers, SSEA-1 and SSEA-4, in pEPSC^{Emb} and pEPSC^{iPS}. c-e. pEPSCs were cultured under seven conditions (ref 9-15) for porcine ESCs previously reported for 7 days, and cell morphology and gene expression were examined. c. Immunofluorescence staining for OCT4 expression. d-e. RT-qPCR detection of OCT4 and NANOG in pEPSCs under each condition. Relative expression levels are shown with normalization to GAPDH. f. Active Oct4 distal enhancer in porcine EPSC^{Emb} and EPSC^{iPS}. The mouse Oct4 distal and proximal enhancer constructs were used in the luciferase assay. Data are mean±s.d. (n=4). g. Genome-editing in pEPSCs^{Emb}. Knocking-in the H2B-mCherry expressing cassette into porcine ROSA26 locus was facilitated by the Crispr/Cas9 system. Out of 20 colonies picked for genotyping, 5 were correctly targeted.

Importantly, the targeted pEPSCs retained a normal karyotype. h. Bright field and fluorescence images of the pEPSC^{Emb} colonies with the H2B-mCherry correctly targeted to the ROSA26 locus. i. in vitro differentiation of pEPSC^{Emb} to cells of the three somatic germ layers and the trophectoderm lineage (KRT7⁺). j. Confocal images of immunostaining SDC1-expressing cells in pEPSC^{Emb} teratoma sections. DAPI stains the nucleus.

Extended Data FIG. 5. In vivo differentiation potential of pEPSCs. a. Participation of pEPSCs in preimplantation embryo development. H2B-mCherry-expressing donor pEPSCs^{iPS} were injected into day 5 host porcine parthenogenetic embryos, which developed to blastocysts. H2BmCherry⁺ donor cells were found in both the inner cell mass and the trophectoderm (arrowed). b. Whole-mount fluorescence and bright field images of 26-day porcine conceptuses derived from preimplantation embryos injected with H2BmCherry⁺ pEPSCs^{Emb}, showing the presence of mCherry⁺ cells in chimera #21. c. Chimeras were processed for two general purposes: half of chimeras were fixed for immunofluorescence analysis, and the other half for FACS and DNA genotyping. To prepare cells for FACS analysis, tissues of each embryo were isolated from head (a), trunk (b) and tail (c), and from the placenta (d), and were dissociated to single cells to detect donor H2BmCherry⁺ cells. The dissociated cells were also used for making genomic DNA samples for PCR analysis. d. PCR genotyping for mCherry DNA using the genomic DNA samples described above. mCherry DNA was only detected in the embryos that were mCherry⁺ by flow cytometry analysis. e. Schematic diagram of day 25-27 porcine chimera conceptuses. The circles mark the tissue areas where tissue sections were taken for immunostaining and imaging as shown below. f. Immunofluorescence analysis of cryosections of day 26-28 mCherry⁺ conceptuses or chimeric embryos and placentas for localisation of H2BmCherry⁺ cells in different tissues. The antibodies used in the analysis include TUJ1 for neurons (Chimera #16); SOX17 and GATA4 for endodermal derivatives (Chimera #21); a-SMA for mesodermal derivatives (Chimera #21); PL-1 and KRT7 for trophoblasts (placenta of Chimera #6), were used. H2BmCherry, GATA4 and SOX17 are found in the nucleus, whereas TUJ, A-SMA, KRT7 and PL-1 are not nuclear localised.

Extended Data FIG. 6. Differentiation of pEPSCs to pPGCLCs. a. Generation of the NANOS3-H2BmCherry reporter EPSCs^{Emb} by targeting the H2B-mCherry cassette to the NANOS3 locus. In the targeted allele, the T2A-H2B-mCherry sequence was in frame with the last coding exon of the porcine NANOS3 locus with the stop codon TAA being deleted. We generated gRNA plasmids targeting specifically to the region covering the NANOS3 stop codon, and 15 colonies were picked for genotyping. Four were correctly targeted. After expansion, those targeted pEPSCs retained a normal karyotype. b. Diagram illustrating the strategy for expressing exogenous genes in pEPSCs^{Emb} for pPGCLC specification and differentiation (see Methods for more details). c. Expressing NANOG, BLIMP1 and TFAP2C individually or in combination with SOX17 in the differentiation of NANOS3-H2BmCherry reporter EPSCs^{Emb} to pPGCLCs (H2BmCherry⁺) in EBs. d. Quantitation of NANOS3-H2BmCherry positive cells in the above (c) experiments. e. RT-qPCR analysis of PGC genes. RNA samples were prepared from day 3 EBs of pEPSCs that expressed transgenes individually or in combinations following the pPGCLC induction protocol in b. Relative expression levels are shown with normalization to GAPDH. Data are mean±s.d. (n=3). Experiments were performed at least three times.

Extended Data FIG. 7. Establishment and characterisation of human EPSCs. a. Images of H1, H9, M1 and M10 human ESC colonies in pEPSCM or in pEPSCM minus ACTIVIN A. Expression of OCT4 was detected by immunostaining. b. Normal karyotype in H1-EPSCs and M1-EPSCs after 25 passages in hEPSCM (10/10 metaphases scored were normal). c. Primary iPSC colony (top) and established cultures of iPSCs (bottom) in hEPSCM reprogrammed from human dermal fibroblasts by Dox-inducible expression of exogenous OCT4, MYC, KLF4, SOX2, LRH1 and RARG. d. Relative expression levels of pluripotency genes (POU5F1, SOX2, NANOG, REX1 and SALL4) in H1-ESCs, H1-naïve ESCs (5i), H1-EPSCs and iPSC-EPSCs. *p<0.05 compared with H1-naïve ESCs (5i), H1-EPSCs and iPSC-EPSCs. Data are mean±s.d. (n=3). e. Detection of potential expression leakiness of the exogenous reprogramming factors by RT-qPCR. No obvious leakiness was found in the four established iPSC lines. f. The relative doubling time of H1-ESCs, H1-naïve ESCs (5i), H1-EPSCs and iPSC-EPSCs. Data are mean±s.d. (n=3). *p<0.05 compared with H1-5i ESCs, H1-EPSCs and iPSC-EPSCs. g. Expression of lineage markers (EOMES, GATA4, GATA6, T, SOX17 and RUNX1) in H1-ESCs, H1-naïve ESCs (5i), H1-EPSCs and iPSC-EPSCs. The primed H1-ESCs had much higher levels of these lineage genes. Data are mean±s.d. (n=3). * p<0.01, gene expression in H1-ESCs compared with H1-5i, H1-EPSCs and iPSC-EPSCs. h. Immunostaining of H1-EPSCs and iPSC-EPSCs for pluripotency factors and cell surface markers. i. In vitro differentiation of H1-EPSCs to the three somatic cell lineages. j. The presence of cartilage (mesoderm. I), glandular epithelium (endoderm. II) and mature neural tissue (glia and neurons, ectoderm. III) by H&E staining in teratomas from hEPSCs in immunocompromised mice. k. EBs of H1-EPSCs to PGCLCs immunostained for SOX17, BLIMP1 and OCT4. l. FACS analysis for expression of CD38 and TNAP on PGCLCs of H1-EPSCs. The induction of PGCLCs was performed on at least two independent human EPSC lines, and experiments were performed at least three times. Relative expression levels are shown with normalization to GAPDH.

Extended Data FIG. 8. RNAseq analysis of human and porcine EPSC transcriptomes. a. Hierarchical clustering of global gene expression data (bulk RNAseq) of human primed and naïve ESCs, human extended pluripotent stem (EPS) cells (Yang, Y, et al, Cell, 2018), and EPSCs of human, porcine and mouse. Correlation matrix was clustered using Spearman correlation and complete linkage. pEPSC^{Par}: EPSC lines from porcine parthenogenetic embryos. E14 and AB2-EPSCs are mouse EPSCs and their RNA-seq data were from our previous publication (Yang, J., et al., Nature, 2017) (ref. 1). The data on human primed ESCs (WIBR1, iPS_NPC_4 and iPS_NPC_13) and naïve ESCs (WIBR2, WIBR3_cl_12, WIBR3_cl_16, WIN1_1 and WIN1_2) were from Theunissen et al, Cell Stem Cell, 2014 and 2016 (Ref 29, and 42). The data of human primed H1 ES cell (H1-rep1 and H1-rep2) and extended pluripotent stem (EPS) cells (H1_EPS_rep1, H1_EPS_rep2, ES1_EPS_rep1 and ES1_EPS_rep2) were from Yang, Y, et al, Cell, 2018 (ref. 43). b-c. Expression of pluripotency and lineage genes in porcine (b) or human (c) EPSCs. d-e. Expression of trophoblast related genes in porcine (d) or human (e) EPSCs.

Extended Data FIG. 9. Epigenetic features of porcine and human EPSCs. a. Global DNA methylation levels in porcine and human EPSCs. H1-5i human naïve ESCs was included in the analysis. Data are mean±s.d. (n=3). *p<0.01, comparison of H1-5i human naïve ESCs with H1-ESCs and H1-EPSCs. b-c. RNAseq analysis of expression of genes encoding enzymes in DNA methylation or demethylation in porcine (b) and human (c) EPSCs. d. PCA of scRNAseq data of human H1-EPSCs and that of human preimplantation embryos (data from Dang Y. et al 2016. Genome Biology. See Methods for more details). e. Violin plots displaying the expression levels of indicated histone genes in human EPSCs (this study) and in human preimplantation embryos at indicated stages (Dang Y. et al 2016. Genome Biology). Gene expression (TPM) was quantified by salmon and the values of log 10(TPM+1). On top of the violin plot, expression in individual cells (represented by dots) was also plotted to show the full distribution of the expression across individual cells. f. Histone modifications (H3K4me3 and H3K27me3) at the loci for genes encoding enzymes involved in DNA methylation and demethylation and for cell lineage genes.

Extended Data FIG. 10. The requirement of individual components in the culture conditions for pEPSCs and hEPSCs. a-b. Effects of removing or adding individual inhibitors on gene expression in pEPSCs^{Emb} (a) and H1-EPSCs (b) analysed by RT-qPCR. “−SRCi, −XAV939, −ACTIVIN, −Vc, −CHIR99”: removing them individually from pEPSCM or hEPSCM; “+TGFβi, +L-CHIR99, +H-CHIR99, +PD03”: adding the TGFβ inhibitor SB431542, a lower concentration of CHIR99021 (0.2 μM, which is the concentration used in pEPSCM), a higher concentration of CHIR99021 (3.0 μM), or three concentrations of MEK1/2 inhibitor PD0325901. WH04/A419 shows the effect of replacing A419259 with another SRC inhibitor, WH-4-23, in human EPSCs. Red triangle indicates no colonies formed. Porcine and human EPSC media contain 0.2 μM and 1.0 μM CHIR99021, respectively. See Methods for medium component information. c. Targeting the OCT4-H2B-Venus cassette into the OCT4 locus in H1-EPSCs. In the targeted allele, the T2A-H2B-Venus sequence was in frame with the last coding exon of the OCT4 gene. The stop codon TGA was deleted. We genotyped 19 colonies, 5 of them were correctly targeted. d. The effects of removing the SRC inhibitor WH-4-023 or XAV939 from hEPSCM for 7 days measured by Venus⁺ cells. The OCT4-H2B-Venus reporter EPSCs were cultured in the indicated conditions and were analysed for Venus expression by fluorescence microscopy and by flow cytometry. e. Western blot analysis of AXIN1 and phosphorylation of SMAD2/3 in porcine and human EPSCs. Both pEPSC^{Emb} and H1-EPSCs had much higher levels of AXIN1. pEPSC^{Emb}, H1-EPSCs and H1-naïve ESCs (5i) had higher levels of TGFβ signalling evidenced by higher pSMAD2/3 than in the differentiated (D) EPSC^{Emb} or primed H1-ESCs. f. TOPflash analysis of the canonical Wnt signalling activities in porcine and human EPSCs. Removing XAV939 from pEPSCM (pEPSCM-X) or hEPSCM (hEPSCM-X) for 5 days substantially increased TOPflash activity. *p<0.01. Data are mean±s.d. (n=4). Experiments were performed at least four times. g. Bright-field and immunofluorescence images showing pEPSCs^{Emb} cultured in pEPSCM or in pEPSCM with the indicated changes in its components. The cells were stained for OCT4 and DAPI. h-i. Quantitation of AP⁺ colonies formed from 2,000 pEPSCs^{Emb} (h) or H1-EPSCs (i) on STO feeders in a 6-well plate by removing medium components or adding small molecule inhibitors. The colonies were scored for 5 consecutive passages to determine the effects of removing XAV939, Vitamin C or CHIR99021, or of using a lower concentration of CHIR99021 (0.2 μM, which is used in pEPSCM), a high concentration of CHIR99021 (3.0 μM), a INK inhibitor, a BRAF inhibitor, or the Mek1/2 inhibitor (PD03). We also quantitated the effect of passaging EPSCs without the ROCK inhibitor Y27632 (−ROCKi). Data are mean±s.d. (n=4) and the experiments were performed three times. j-k. RT-qPCR analysis of expression of lineage genes in pEPSCs^{Emb} (j) or hEPSCs (k), when XAV939 or ACTIVIN A was removed from pEPSCM and hEPSCM, or when TGFβ signalling was inhibited by SB431542. The effect of 3.0 μM CHIR99021 was also analysed. 1. The effects of supplementing 5.0 ng/ml ACTIVIN A in hEPSCM on the expression of lineage genes in EBs formed from H1-EPSCs. Expression of genes of mesendoderm lineage was substantially increased. *p<0.05 comparison to human EPSCs cultured supplemented with ACTIVIN A. m-n. Differentiation to PGCLCs from the NANOS3-Tdtomato reporter EPSCs cultured in hEPSCM either with or without 5.0 ng/ml ACTIVIN A. Adding ACTIVIN A substantially increased PCGLCs measured in FACS (Tdtomato⁺). RT-qPCR analysis of PGCLC genes confirmed the increase of PCGLCs. *p<0.05 in comparison to hEPSCM supplemented with ACTIVIN A. RT-qPCR data are mean±s.d. (n=3). Experiments were performed at least three times. Relative expression levels are shown with normalization to GAPDH.

Extended Data FIG. 11. Characterization of hEPSC trophoblast differentiation potential. a. Generation of the CDX2-H2BVenus reporter EPSC line. In the targeted allele, the T2A-H2BVenus sequence was in frame with the last coding exon of the human CDX2 gene. The TGA stop codon was deleted in the targeted allele. The reporter EPSCs were subsequently cultured in hEPSCM, in the standard FGF-containing human ESC medium or in the 5i condition for human naïve ESCs, for subsequent analyses. b. Trophoblast gene expression measured by RT-qPCR in cells induced to differentiate to trophoblasts by 4-day BMP4 treatment. Experiments were performed at least three times. Data are mean±s.d. (n=3). *p<0.01 compared with H1-ESCs and H1-5i naïve cells. c. Trophoblast gene expression measured by RT-qPCR in hEPSC induced to differentiate to trophoblasts by SB431542+PD173074+BMP4. Cells were collected at several time points for analysis. qRT-PCR data are mean±s.d. (n=3). Relative expression levels are shown with normalization to GAPDH. d. Heatmap shows expression changes of trophoblast genes in cells differentiated from H1-ESCs (green), H1-EPSCs (red) or iPSC-EPSCs (blue) (RNAseq data are in Supplementary Table 6). Cells were collected at several differentiation time points for RNAseq analysis. e. Pearson correlation coefficient of gene expression in cells differentiated from H1-ESCs, H1-EPSCs and iPSC-EPSCs (RNAseq data in Supplementary Table 6), with the published data of PHTu and PHTd (undifferentiated and differentiated human primary trophoblasts, respectively) and with human tissues. The details of these analyses are given in Methods. f. Detection of the four C19MC miRNAs (hsa-miR-525-3p, -526b-3p, -517-5p, and -517b-3p) in cells differentiated from H1-EPSCs, H1-ESCs, H1-naïve ESCs (5i) and iPSC-EPSCs treated with SB431542 for six days. The choriocarcinoma cells JEG-3 that are representatives of extravillous trophoblasts, and JAR that are representatives of villous trophoblast cells, were used as the control. g. The expressions of the same four miRNAs as presented above in the BMP4 (4-day) treated human EPSCs and human ESCs. Data are mean±s.d. (n=3). *p<0.05 compared with H1-ESCs. Relative miRNAs expression levels are shown with normalization to miR-103a. h. DNA demethylation in the promoter region of the ELF5 locus in cells differentiated from H1-EPSCs and other cells (6 days of SB431542 treatment). Cells from H1-ESCs, H1-naïve ESCs (5i) did not have substantial DNA demethylation at the ELF5 promoter. i. Secreted hormones from trophoblasts derived from H1-EPSCs induced by TGFβ inhibition (SB431542). VEGF, PLGF, sFlt-1 and sEng were measured in the conditioned media of cells differentiated from EPSCs or ESC cultures upon SB431542 treatment over a 48 h interval until day 16. j. hCG secreted from trophoblasts from EPSCs or ESCs. hCG secreted from day-10 differentiated (SB431542 treatment) EPSCs and ESCs were measured by ELISA. Data are mean±s.d. (n=4). *p<0.01 compared with H1-ESC.

Extended Data FIG. 12. Derivation and characterisation of trophoblast stem cell-like cells (hTSCs) from human EPSCs. a. RT-qPCR analysis of pluripotency and trophoblast stem cell genes in four EPSC-derived TSC lines and their parental hEPSCs. Data are mean±s.d. (n=3). *p<0.01 compared to TSCs. b. PCA of gene expression of hTSCs derived from EPSCs and of cells differentiated from H1-EPSCs treated with TGFβ inhibitor SB431542 at several time points. hTSCs appear to have enriched transcriptomic features of day-4 differentiated EPSCs. c. Phase-contrast and Hoechst staining images of multinucleated syncytiotrophoblasts differentiated from TSCs. d. Immunofluorescence detection of CGB in syncytiotrophoblasts differentiated from TSCs derived from hESPCs. e. Efficiency of forming syncytiotrophoblasts from hTSCs. The fusion index is calculated as the number of nuclei in syncytial/total number of nuclei. Data are presented as mean±SD (n=4). *p<0.01 compared to TSCs. f. RT-qPCR analysis of trophoblast genes in three TSC lines and their derivative syncytiotrophoblast (ST) and extravillous trophoblast (EVT). Relative expression levels are shown with normalization to GAPDH. g. Detection of HLA class I by monoclone antibody W6/32 in undifferentiated hESCs, hEPSCs, hTSCs, and in hEVT differentiated from hTSCs. Compared to hESCs, hEPSCs and hTSCs expressed substantially lower levels of HLA class I molecules. EVTs are known to express HLA-C. The choriocarcinoma cells JEG-3 and JAR are representatives of extravillous and villous trophoblast cells, respectively. JEG-3 express HLA-G, HLA-C and HLA-E, whereas JAR cells do not express any HLA molecules (Apps, R., et al. Immunology 2009). They were used as the positive and negative control, respectively. h. The isotype control for HLA-G flow cytometry analysis related to FIG. 4g. i. H&E staining of lesions formed from subcutaneously injected hTSCs in NOD-SCID mice. j. Serum hCG levels in 6 NOD-SCID mice 7 days after the mice were subcutaneously injected with hTSCs (n=3) or vehicle control (n=3).

Extended Data FIG. 13. Derivation and characterisation of trophoblast stem cell-like cells (pTSCs) from porcine EPSCs. a. H3K27me3 and H3K4me3 marks at the loci encoding factors associated with placenta development in pEPSC^{Emb} and human H1-EPSCs. b. Images of primary TSC colonies (top) formed from individual pEPSC^{Emb} on day 7 cultured in human TSC condition, and of established pTSCs at passage 7 (bottom). Dashed lines mark the area of putative trophoblasts, which were picked for establishing stable pTSC lines. c. RT-qPCR analysis of pluripotency and trophoblast genes in four pTSC lines and their parental pEPSC^{Emb}. Data are mean±s.d. (n=3). *p<0.01 comparison between pEPSCs to pTSCs. Relative expression levels are shown with normalization to GAPDH. d. Expression of trophoblast factors GATA3 and KRT7 in pEPSC^{Emb}-TSCs detected by immunostaining. Nuclei were stained with DAPI. e. Confocal image of immunostaining of sections of lesions formed from pTSCs in NOD-SCID mice for cells expressing SDC1 and KRT7. f. H&E staining of sections of the lesions formed when pTSCs were subcutaneously injected to immunocompromised mice. g. Confocal images of immunostaining of porcine blastocysts 1 to 2 days following injection of pTSCs. H2B-mCherry-expressing pTSCs were injected into porcine parthenogenetic morulae and early blastocysts (n=50 blastocysts in two injections). Arrows indicate H2B-mCherry⁺ cells in the TE which expressed the porcine trophectoderm transcription factor CDX2 and GATA3.

Extended Data FIG. 14. The effects of inactivation of PARG in human EPSCs on trophoblast differentiation potential. a. CRISPR/Cas9 mediated deletion of ˜350 bp in exon 4 of the PARG gene in the CDX2-H2BVenus reporter hEPSCs. Two gRNAs (g1, g2) were designed to target the largest coding exon. After transfection and selection, 6 clones out 48 clones were identified as bi-allelic mutants by PCR genotyping and were confirmed by sequencing. b. The CDX2-reporter EPSC cells with or without the PARG deletion were treated with the TGFβ inhibitor SB431542 for four days for trophoblast differentiation. The cells were analysed by flow cytometry. c. The percentages of Venus⁺ cells indicate the extent of trophoblast differentiation of the parental cells. Inactivation of PARP caused decreased Venus⁺ cells. Data are mean±s.d. (n=3). *p<0.05 comparison between wide type and PARG^{-/-} H1-EPSCs. Similar results were obtained in experiments using two independent PARP-deficient human EPSC lines. d. RT-qPCR analysis of expression of trophoblast genes in cells differentiated from either the control (wild type) or the PARG-deficient CDX2-H2BVenus H1-EPSCs, after 6 days of SB431542 treatment. Significantly lower trophoblast gene expression was found in the PARG-deficient cells. *p<0.05. Data are mean±s.d. (n=3). Relative expression levels are shown with normalization to GAPDH. Experiments were performed at least three times.

### 4.2 Definitions

Unless defined otherwise, all technical and scientific terms used herein have the same meaning as commonly understood by those of ordinary skill in the art to which this disclosure belongs. Although any methods and materials similar or equivalent to those described herein can be used in the practice or testing of the present disclosure, the preferred methods and materials are described. For purposes of the present disclosure, the following terms are defined below.

“iPSCs” are pluripotent cells which are derived from non-pluripotent, differentiated ancestor cells. Suitable ancestor cells include somatic cells, such as adult fibroblasts and peripheral blood cells. These ancestor cells are typically reprogrammed by the introduction of pluripotency genes (or RNA encoding them) or their corresponding proteins into the cell, or by re-activating the endogenous pluripotency genes. The introduction techniques include plasmid or viral transfection or direct protein delivery in certain embodiments.

“Feeder cells” or “feeders” are terms used to describe cells of one type that are co-cultured with cells of another type, to provide an environment in which the cells of the second type can grow. A feeder free culture will contain less than about 5% feeder cells. Compositions containing less than 1%, 0.2%, 0.05%, or 0.01% feeder cells (expressed as % of total cells in the culture) are increasingly more preferred.

A “growth environment” is an environment in which cells of interest will proliferate in vitro. Features of the environment include the medium in which the cells are cultured, and a supporting structure (such as a substrate on a solid surface) if present.

A “nutrient medium” is a medium for culturing cells containing nutrients that promote proliferation, including: isotonic saline, buffer, amino acids, serum or serum replacement, and other exogenously added factors.

A “conditioned medium” is prepared by culturing a first population of cells in a medium, and then harvesting the medium. The conditioned medium, along with anything secreted into the medium by the cells, may then be used to support the growth of a second population of cells. Where a particular ingredient or factor is described as having been added to the medium, the factor has been mixed into the medium by deliberate manipulation.

The term “antibody” as used in this disclosure refers to both polyclonal and monoclonal antibody of any species. The ambit of the term encompasses not only intact immunoglobulin molecules, but also fragments and genetically engineered derivatives of immunoglobulin molecules and equivalent antigen binding molecules that retain the desired binding specificity.

The terms “isolated” or “purified” refer to material that is substantially or essentially free from components that normally accompany it as found in its native state. Purity and homogeneity are typically determined using analytical chemistry techniques such as polyacrylamide gel electrophoresis or high performance liquid chromatography.

The term “serum” as used herein means the liquid portion of the blood that remains after blood cells and fibrinogen/fibrin are removed. The term “serum-free culture medium” means a culture medium containing no serum or product extracted from sera of animals and especially those originating from mammals, birds, fish or crustaceans.

The term “comprising” encompasses “including” as well as “consisting” e.g. a composition “comprising” X may consist exclusively of X or may include something additional e.g. X+Y.

Unless otherwise indicated by the terms “exactly”, “precisely”, or another equivalent term, all numbers expressing quantities of ingredients, properties such as molecular weight, reaction conditions, and so forth as used herein, are to be understood as being modified in all instances by the term “about”, and thus to inherently include variations of up to 10% greater or less than the actual number stated. Accordingly, the numerical parameters herein are approximations depend upon the desired properties sought to be obtained by the present disclosure. At the very least, each numerical parameter should at least be construed given the number of reported significant digits and by applying ordinary rounding techniques. Notwithstanding that the numerical ranges and parameters describing the broad scope of the disclosure are approximations, the numerical values in the specific examples are reported as precisely as possible. Any numerical value, however, inherently contains standard deviations that necessarily result from the errors found in the numerical value's testing measurements.

### 5. DETAILED DESCRIPTION

Described herein is the production of expanded potential stem cells (EPSCs) from populations of pluripotent cells. EPSCs have ‘naïve’ or ground state properties and have an expanded potential to differentiate into extraembryonic cell lines (trophoblasts and extraembryonic endoderm in the yolk sac) as well as cells of the embryo proper. EPSCs may be produced from different pluripotent cell lines which are cultured in expanded potential stem cell media (EPSCM). EPSCs have been successfully differentiated into a range of cell types including somatic cells and trophoblast cells. EPSCs may be useful for studying the mechanisms of development and EPSCs or cells differentiated therefrom. This helps particularly with research and R&D in regenerative medicine, for example in disease modelling, screening for therapeutics, testing toxicity, studying genetic diseases and studying reproductive biology.

A population of expanded potential stem cells (EPSCs) may be produced by culturing a population of pluripotent cells (PSCs) in an expanded potential stem cell medium (EPSCM) to produce a population of EPSCs. Described herein is the derivation of porcine EPSC (pEPSC) lines either directly from preimplantation embryos or by reprogramming porcine fetal fibroblasts. Pluripotent cells may include embryonic stem cells (ESCs) and non-embryonic stem cells, for example fetal and adult stem cells, and induced pluripotent stem cells (iPSCs).

### 5.1 New Porcine iPSC Generation

While porcine iPSCs are available, the use of these cells for the screen is confounded by the leaky expression of the transgenic reprogramming factors after reprogramming or by low levels of expression of the endogenous pluripotency genes [11-19]. To overcome this challenge, new porcine iPSCs are generated to express pluripotency genes such as Doxycycline (Dox)-inducible LIN28, NANOG, LRH1 and RARG, in concert with the four Yamanaka factors.

The pluripotency genes or proteins may comprise one, two, three, four, five or six of a LIN family member, NANOG family member, LRH family member, RAR family member.

The Lrh family member may be LRH1.

The Rar family member may be Rar-g.

In one embodiment, pluripotency genes or proteins may comprise Oct4, Sox2, Klf4 and c-Myc (Yamanaka factors).

Techniques for the production of iPSCs are well-known in the art (Yamanaka et al Nature 2007; 448:313-7; Yamanaka 6 2007 Jun. 7; 1(1):39-49; Kim et al Nature. 2008 Jul. 31; 454(7204):646-50; Takahashi Cell. 2007 Nov. 30; 131(5):861-72. Park et al Nature. 2008 Jan. 10; 451(7175):141-6; Kimet et al Cell Stem Cell. 2009 Jun. 5; 4(6):472-6; Dallier, L., et al. Stem Cells, 2009. 999(999A), Wang W, et al. PNAS. (2011) 108; 45; 18283-8. However, the strategy provided herein substantially improves the efficiency of reprogramming wild-type German Landrace porcine fetal fibroblasts (PFFs) and transgenic PFFs, in which a tdTomato cassette had been inserted into the 3′ UTR of the porcine OCT4 (POU5F1) locus (POT PFFs) [20], to putative iPSC colonies (Extended Data FIG. 1a-c). The reprogrammed primary colonies from POT PFFs were OCT4-tdTomato⁺, indicating the re-activation of the OCT4 locus (Extended Data FIG. 1c). Indeed, RT-qPCR revealed that the iPSCs expressed high levels of the endogenous pluripotency factors (Extended Data FIG. 1d), and could be passaged as single cells on STO feeders for more than 20 passages in serum-containing medium (M15) plus Dox.

Upon Dox removal, the iPSCs differentiated within 4-5 days, concomitant with rapid down-regulation of the exogenous reprogramming factors and endogenous pluripotency genes and with increased expression of both embryonic and extraembryonic cell lineage genes (Extended Data FIG. 1e-h). These Dox-dependent porcine iPSCs with robust endogenous pluripotency gene expression provided the material for the chemical screen.

Thus, a population of pluripotent stem cells may be obtained by reprogramming non-pluripotent cells, such as somatic cells into induced pluripotent stem cells (iPSCs) by introducing pluripotency genes or their corresponding proteins, or by reactivating the endogenous pluripotency genes, using techniques which are known in the art and discussed herein.

The iPSCs may be obtained from a mammalian individual. Mammals include canines, felines, rodents, bovine, equines, porcines, ovines, and primates. Avians include, but are not limited to, fowls, songbirds, and raptors. In some embodiments, the iPSCs may be derived from somatic cells or other antecedent cells obtained from an individual. The iPSCs may be used to produce a population of EPSCs which share the genotype of that individual. In some embodiments the EPSCs or cells differentiated therefrom in vitro produced from an individual, may be useful in studying the mechanisms of a disease condition associated with that individual.

### 5.2 Culture Media

Suitable culture media for pluripotent cells are well-known in the art and include; Knockout Dulbecco's Modified Eagle's Medium (KO-DMEM) supplemented with 20% Serum Replacement, 1% Non-Essential Amino Acids, 1 mM L-Glutamine, 0.1 mM 0-mercaptoethanol and 4 ng/ml to 10 ng/ml FGF2; or Knockout (KS) medium supplemented with 4 ng/ml FGF2; or KO-DMEM supplemented with 20% Serum Replacement, 1% Non-Essential Amino Acids, 1 mM L-Glutamine, 0.1 mM (3-mercaptoethanol and 4 ng/ml to 10 ng/ml human FGF2; or DMEM/F12 supplemented with 20% knockout serum replacement (KSR), 6 ng/ml FGF2 (PeproTech), 1 mM L-Gln, 100 μm non-essential amino acids, 100 μM 2-mercaptoethanol, 50 U/ml penicillin and 50 mg/ml streptomycin.

In certain embodiments, a population of pluripotent cells for use in the present methods may be cultured in a chemically defined medium (CDM) which comprise a chemically defined basal medium comprising inhibitors for GSK3 (CHER99021), SRC (WH-4-023) and Tankyrases (XAV939) (the last two were inhibitors important for mouse EPSCs[1]) (#517, porcine EPSC medium: pEPSCM) (Extended Data FIG. 2h), also supplemented with one or more additional components, for example Vitamin C (Vc), ACTIVIN A and LIF (Extended Data FIG. 2a, 2h and Supplementary Table 1). Under these conditions, the Dox-independent iPSCs (pEPSC^{iPS}) remained undifferentiated in 30 passages, expressed endogenous pluripotency factors at levels comparable to the porcine blastocyst and showed no leaky expression of the exogenous reprogramming factors (Extended Data FIG. 3b-d).

To maintain Dox-independent porcine iPSCs in the undifferentiated state (Extended Data FIG. 2a; Supplementary Table 1), inhibitors of Mek1, p38 and PKC are excluded after screening over 400 combinations of 20 small molecule inhibitors and cytokines for their ability to maintain putative porcine iPSCs. Distinction from previous reports using mouse model was reported; naïve mouse ESC medium 2i/LIF was able to maintain putative porcine iPSCs [15, 17, 21], but porcine iPSCs were rapidly lost in the presence of the Mek1 inhibitor PD-0325901 at 1.0 μM, irrespective of whether Dox was present or not (Extended Data FIG. 2b-h). This indicates that porcine pluripotent stem cells and mouse ESCs differ in the requirement of Mek-ERK signaling. [26-28] Inhibition of p38 and PKC was also nonconducive for porcine iPSCs (Extended Data FIG. 2b-h and Extended Data FIG. 3a). These findings led conclusion that mouse or human naïve ESC conditions [22-24] cannot be directly extrapolated to porcine pluripotent stem cells. These three inhibitors for Mek1/2, p38 and PKC were therefore excluded from the screen.

Suitable techniques for cell culture are well-known in the art (see, for example, Basic Cell Culture Protocols, C. Helgason, Humana Press Inc. U.S. (15 Oct. 2004) ISBN: 1588295451; Human Cell Culture Protocols (Methods in Molecular Medicine S.) Humana Press Inc., U.S. (9 Dec. 2004) ISBN: 1588292223; Culture of Animal Cells: A Manual of Basic Technique, R. Freshney, John Wiley & Sons Inc (2 Aug. 2005) ISBN: 0471453293, Ho W Y et al J Immunol Methods. (2006) 310:40-52, Handbook of Stem Cells (ed. R. Lanza) ISBN: 0124366430) Basic Cell Culture Protocols' by J. Pollard and J. M. Walker (1997), ‘Mammalian Cell Culture: Essential Techniques’ by A. Doyle and J. B. Griffiths (1997), ‘Human Embryonic Stem Cells’ by A. Chiu and M. Rao (2003), Stem Cells: From Bench to Bedside’ by A. Bongso (2005), Peterson & Loring (2012) Human Stem Cell Manual: A Laboratory Guide Academic Press and ‘Human Embryonic Stem Cell Protocols’ by K. Turksen (2006). Media and ingredients thereof may be obtained from commercial sources (e.g. Gibco, Roche, Sigma, Europa bioproducts, R&D Systems). Standard mammalian cell culture conditions may be employed for the above culture steps, for example 37° C., 5% Carbon Dioxide.

A population of pluripotent cells for use may be cultured in the present expanded potential stem cell medium (EPSCM) described herein to produce a population of EPCSs. Once converted, the EPSCs may be cultured in an EPSC maintenance medium (EPSCMM). The maintenance medium may have a composition as described herein, for example, fewer inhibitors/modulators compared to the EPSCM which was used for converting the cells. Once converted, EPSCs may not require as many inhibitors/modulators to maintain them in culture as EPSCs.

A suitable porcine EPSCM of 500 ml comprise one or more:

0.3 μM WH-4-023 (SRC inhibitor, TOCRIS, Cat. No. 5413),

2.5 μM XAV939 (Sigma, Cat. No. X3004) or 2.0 μM IWR-1 (TOCRIS, Cat. No. 3532),

50 μg/ml Vitamin C (Sigma, Cat. No. 49752-100G),

10 ng/ml LIF (Stem Cell Institute, University of Cambridge. SCI),

20 ng/ml ACTIVIN (SCI).

Optionally the EPSCM may also contain LIE The EPSCM may contain a nutrient medium.

A suitable EPSCM or EPSCMM comprise nutrient medium and a GSK3 inhibitor.

A suitable EPSCM or EPSCMM may contain one or more of the following ingredients: 482.5 ml DMEM/F-12 (Gibco, Cat. No. 21331-020), 2.5 ml N2 supplement (Thermo Fisher Scientific, Cat. No. 17502048), 5 ml B27 supplement (Thermo Fisher Scientific, Cat. No. 17504044), 5 ml 1× Glutamine Penicillin-Streptomycin (Thermo Fisher Scientific, Cat. No. 11140-050), 5 ml 1×NEAA (Thermo Fisher Scientific, Cat. No. 10378-016), 110 μM 2-mercaptoethanol (Sigma, Cat. No. M6250), and 0.2 μM CHIR99021(GSK3i, TOCRIS, Cat. No. 4423), 0.3% FBS (Gibco, Cat. No. 10270).

A suitable porcine EPSCM of 500 ml comprise one or more of the following ingredients:

ITS-X 200× (thermos, 51500056), add 2.5 ml;

Vitamin C(Sigma, 49752-100G), working concentration 64 μg/ml;

Bovine Albumin Fraction V (7.5% solution) (Thermo, 15260037), 3 ml;

Trace elements B(Corning, MT99175CI) 1000×

Trace elements C(Corning, MT99176CI) 1000×

reduced glutathione(sigma, G6013-5G) 10 mg/ml, add 165 ul

XAV939 (Sigma X3004), working concentration 2.5 μM;

endo-IWR-1(Tocris, Cat. No. 3532), working concentration 1 μM

WH-4-023 (Tocris, Cat. No. 5413), working concentration 0.16 μM;

Chiron 99021 (Tocris Bioscience, 4423), working concentration 0.2 μM;

Human Lif, working concentration 10 ng/ml; and

Activin A(S TEM CELL TECHNOLOGY, Catalog #78001.1) 20 ng/ml.

A suitable EPSCM or EPSCMM may contain one or more of the following ingredients: F12 DMEM (Gibco, 21331-020), add 240 ml; Neurobasal medium (Life Technologies, 21103-049) 240 ml; Penicillin-Streptomycin-Glutamine (100×) (Gibco, 10378016), add 5 ml; NEAA 100× (Gibco, 11140050), add 5 ml; Sodium Pyruvate100× (gibco, 11360070), add 5 ml; 14.3M 2-Mercaptoethanol (M6250 Aldrich, Sigma), add 3.8 μl (working concentration 110 μM); 200×N2 (Thermo 17502048), add 2.5 ml; and 100×B27 (Thermo 17504044), add 5 ml.

A suitable human EPSCM of 500 ml comprise one or more of the following ingredients:

0.1 μM A-419259 (SRC inhibitor, TOCRIS, Cat. No. 3914),

2.5 μM XAV939 (Sigma, Cat. No. X3004) or 2.5 μM IWR-1 (TOCRIS, Cat. No.

3532),

50 μg/ml Vitamin C (Sigma, Cat. No. 49752-100G),

10 ng/ml LIF (SCI).

Optionally the EPSCM may also contain LIF. The EPSCM may contain a nutrient medium.

A suitable EPSCM or EPSCMM comprise a nutrient medium together with a GSK3 inhibitor.

A suitable EPSCM or EPSCMM may contain one or more of the following ingredients: 482.5 ml DMEM/F-12 (Gibco, Cat. No. 21331-020), 2.5 ml N2 supplement (Thermo Fisher Scientific, Cat. No. 17502048), 5 ml B27 supplement (Thermo Fisher Scientific, Cat. No. 17504044), 5 ml 1× Glutamine Penicillin-Streptomycin (Thermo Fisher Scientific, Cat. No. 11140-050), 5 ml 1×NEAA (Thermo Fisher Scientific, Cat. No. 10378-016), 110 μM 2-mercaptoethanol (Sigma, Cat. No. M6250), and 1.0 μM CHIR99021(GSK3 inhibitor, TOCRIS, Cat. No. 4423).

A suitable human EPSCM of 500 ml may comprise one or more of the following ingredients:

ITS-X 200× (thermos, 51500056), add 2.5 ml

Vitamin C (Sigma, 49752-100G), working concentration 64 μg/ml;

Bovine Albumin Fraction V (7.5% solution) (Thermo, 15260037), 3 ml;

Trace elements B (Corning, MT99175CI) 1000×

Trace elements C (Corning, MT99176CI) 1000×

reduced glutathione (sigma, G6013-5G) 10 mg/ml, add 165 μl

defined lipids (Invitrogen, 11905031) 500×

XAV939 (Sigma X3004), working concentration 2.5 μM;

endo-IWR-1(Tocris, Cat. No. 3532), working concentration 2.5 μM

A419259 (Tocris Bioscience, 3748), working concentration 0.1 μM;

Chiron 99021 (Tocris Bioscience, 4423), working concentration 1.0 μM.

A suitable EPSCM or EPSCMM may contain one or more of the following ingredients: F12 DMEM (Gibco, 21331-020), add 240 ml; Neurobasal medium (Life Technologies, 21103-049) 240 ml; Penicillin-Streptomycin-Glutamine (100×) (Gibco, 10378016), add 5 ml; NEAA 100× (Gibco, 11140050), add 5 ml; Sodium Pyruvate100×(gibco, 11360070), add 5 ml; 14.3M 2-Mercaptoethanol (M6250 Aldrich, Sigma), add 3.8 μl (working concentration 110 μM); 200×N2 (Thermo 17502048), add 2.5 ml; 100×B27 (Thermo 17504044), add 5 ml; and Human Lif, working concentration 10 ng/ml.

In one embodiment, porcine EPSC media comprises:

DMEM/F-12 (Gibco, Cat. No. 21331-020), or knockout DMEM (Gibco, Cat. No. 10829-018), basal media, 98%;

N2 supplement (Thermo Fisher Scientific, Cat. No. 17502048), range from 0.1 to 1%, between 0.25 to 0.75%, between 0.4-0.6%;

B27 supplement (Thermo Fisher Scientific, Cat. No. 17504044), range from 0.1 to 2%, between 0.5 to 1.5%, between 0.8-1.0%;

Glutamine Penicillin-Streptomycin (Thermo Fisher Scientific, Cat. No. 11140-050), basal supplement, 1%;

NEAA (Thermo Fisher Scientific, Cat. No. 10378-016), basal supplement, 1%;

2-mercaptoethanol (Sigma, Cat. No. M6250), basal supplement, 110 μM;

CHIR99021(GSK3i, TOCRIS, Cat. No. 4423), range from 0.05 to 0.5 μM, between 0.1 to 0.5 μM, between 0.2 to 0.3 μM;

WH-4-023 (SRC inhibitor, TOCRIS, Cat. No. 5413), range from 0.1 to 1.0 μM, between 0.2 to 0.8 μM, between 0.3 to 0.5 μM;

XAV939 (Sigma, Cat. No. X3004), range from 1 to 10 μM, between 2 to 5 μM, even between 2.5 to 4.5 μM; or IWR-1 (TOCRIS, Cat. No. 3532), range from 1 to 10 μM, between 2 to 5 μM, between 2.5 to 4.5 μM;

Vitamin C (Sigma, Cat. No. 49752-100G), range from 10 to 100 μg/ml, between 20 to 80 μg/ml, between 50 to 70 μg/ml;

LIF (Stem Cell Institute, University of Cambridge. SCI), range from 1 to 20 ng/ml, between 5 to 15 ng/ml, between 8 to 12 ng/ml;

ACTIVIN (SCI), range from 10 to 50 ng/ml, between 15 to 30 ng/ml, even between 20 to 25 ng/ml;

FBS (Gibco, Cat. No. 10270) range from 0.1 to 0.5%, preferably between 0.2 to 0.4%, between 0.25-0.35% and

ITS-X (thermos, 51500056), range from 0.1 to 2%, preferably between 0.2 to 0.8%, between 0.4-0.6%.

In another embodiment, human EPSC media comprises:

DMEM/F-12 (Gibco, Cat. No. 21331-020), or knockout DMEM (Gibco, Cat. No. 10829-018), basal media, 98%;

N2 supplement (Thermo Fisher Scientific, Cat. No. 17502048), range from 0.1 to 1%, between 0.25 to 0.75%, between 0.4-0.6%;

B27 supplement (Thermo Fisher Scientific, Cat. No. 17504044), range from 0.1 to 2%, between 0.5 to 1.5%, between 0.8-1.0%;

Glutamine Penicillin-Streptomycin (Thermo Fisher Scientific, Cat. No. 11140-050), basal supplement, 1%;

NEAA (Thermo Fisher Scientific, Cat. No. 10378-016), basal supplement, 1%;

2-mercaptoethanol (Sigma, Cat. No. M6250), basal supplement, 110 μM;

CHIR99021(GSK3 inhibitor, TOCRIS, Cat. No. 4423), range from 0.2 to 2 μM, between 0.5 to 1.5 μM, between 0.8 to 1.2 μM;

A-419259 (SRC inhibitor, TOCRIS, Cat. No. 3914), range from 0.05 to 0.5 μM, between 0.1 to 0.5 μM, between 0.15 to 0.3 μM XAV939 (Sigma, Cat. No. X3004) range from 1 to 10 μM, between 2 to 5 μM, between 2.5 to 4.5 μM or IWR-1 (TOCRIS, Cat. No. 3532), range from 1 to 10 μM, between 2 to 5 μM, between 2.5 to 4.5 μM;

Vitamin C (Sigma, Cat. No. 49752-100G), range from 10 to 100 μg/ml, between 20 to 80 μg/ml, between 50 to 70 μg/ml;

LIF (SCI), range from 1 to 20 ng/ml, between 5 to 15 ng/ml, between 8 to 12 ng/ml;

In another embodiment, human EPSC media comprises:

F12 DMEM (Gibco, 21331-020), basal media, 48%

Neurobasal medium (Life Technologies, 21103-049), basal media, 48%

Penicillin-Streptomycin-Glutamine (Gibco, 10378016), basal supplement, 1%

NEAA (Gibco, 11140050), basal supplement, 1%

Sodium Pyruvate (gibco, 11360070), basal supplement, 1%

2-Mercaptoethanol (Aldrich, Sigma), basal supplement, 110 μM

N2 (Thermo 17502048), range from 0.1 to 1%, between 0.25 to 0.75%, between 0.4-0.6%

B27 (Thermo 17504044), range from 0.1 to 2%, between 0.5 to 1.5%, between 0.8-1.0%

ITS-X (thermos, 51500056), range from 0.1 to 1%, between 0.25 to 0.75%, between 0.4-0.6%

Vitamin C (Sigma, 49752-100G), range from 10 to 100 μg/ml, between 20 to 100 μg/ml, between 50 to 70 μg/ml

Bovine Albumin Fraction V (7.5% solution) (Thermo, 15260037), range from 0.1% to 1%, between 0.2 to 0.8%, between 0.4-0.6%

trace elements B (Corning, MT99175CI) basal supplement, 0.1%

trace elements C (Corning, MT99176CI) basal supplement, 0.1%

reduced glutathione (sigma, G6013-5G) range from 1 to 20 μg/ml, between 1 to 10 μg/ml, between 2 to 5 μg/ml

defined lipids (Invitrogen, 11905031) basal supplement, 0.2%

XAV939 (Sigma X3004), range from 1 to 10 μM, between 2 to 5 μM, between 2.5 to 4.5 μM

endo-IWR-1(Tocris, Cat. No. 3532), range from 1 to 10 μM, between 2 to 5 μM, between 2.5 to 4.5 μM

A419259 (Tocris Bioscience, 3748), range from 0.05 to 0.5 μM, between 0.1 to 0.5 μM, between 0.15 to 0.3 μM

Chiron 99021 (Tocris Bioscience, 4423), range from 0.2 to 2 μM, between 0.5 to 1.5 μM, between 0.8 to 1.2 μM

Human Lif(Stem Cell Institute, University of Cambridge. SCI), range from 1 to 20 ng/ml, between 5 to 15 ng/ml, between 8 to 12 ng/ml

In one embodiment, Porcine EPSC media comprises:

F12 DMEM (Gibco, 21331-020), basal media, 48%

Neurobasal medium (Life Technologies, 21103-049), basal media, 48%

Penicillin-Streptomycin-Glutamine (Gibco, 10378016), basal supplement, 1%

NEAA (Gibco, 11140050), basal supplement, 1%

Sodium Pyruvate (gibco, 11360070), basal supplement, 1%

2-Mercaptoethanol (Aldrich, Sigma), basal supplement, 110 μM

N2 (Thermo 17502048), range from 0.1 to 1%, between 0.25 to 0.75%, between 0.4-0.6%

B27 (Thermo 17504044), range from 0.1 to 2%, between 0.5 to 1.5%, between 0.8-1.0%

ITS-X (thermos, 51500056), range from 0.1 to 1%, between 0.25 to 0.75%, between 0.4-0.6%

Vitamin C (Sigma, 49752-100 G), range from 10 to 100 μg/ml, between 20 to 100 μg/ml, between 50 to 70 μg/ml

Bovine Albumin Fraction V (7.5% solution) (Thermo, 15260037), range from 0.1% to 1%, between 0.2 to 0.8%, between 0.4-0.6%

trace elements B (Corning, MT99175CI) basal supplement, 0.1%

trace elements C (Corning, MT99176CI) basal supplement, 0.1%

reduced glutathione (sigma, G6013-5G) range from 1 to 20 μg/ml, between 1 to 10 μg/ml, between 2 to 5 μg/ml

XAV939 (Sigma X3004), range from 1 to 10 μM, between 2 to 5 μM, between 2.5 to 4.5 μM

endo-IWR-1 (Tocris, Cat. No. 3532), range from 1 to 10 μM, between 1 to 5 μM, between 1 to 2 μM

WH-4-023 (Tocris, Cat. No. 5413), range from 0.1 to 1.0 μM, between 0.1 to 0.5 μM, between 0.1 to 0.2 μM

Chiron 99021 (Tocris Bioscience, 4423), range from 0.05 to 0.5 μM, between 0.1 to 0.5 μM, between 0.2 to 0.3 μM

Human Lif(Stem Cell Institute, University of Cambridge. SCI), range from 1 to 20 ng/ml, between 5 to 15 ng/ml, between 8 to 12 ng/ml

Activin A (STEM CELL TECHNOLOGY, Catalog #78001.1). range from 10 to 50 ng/ml, between 15 to 30 ng/ml, between 20 to 25 ng/ml.

In one embodiment, 500 ml porcine EPSC media comprises:

482.5 ml DMEM/F-12 (Gibco, Cat. No. 21331-020),

2.5 ml N2 supplement (Thermo Fisher Scientific, Cat. No. 17502048),

5 ml B27 supplement (Thermo Fisher Scientific, Cat. No. 17504044),

5 ml 1× Glutamine Penicillin-Streptomycin (Thermo Fisher Scientific, Cat. No. 11140-050),

5 ml 1×NEAA (Thermo Fisher Scientific, Cat. No. 10378-016),

110 μM 2-mercaptoethanol (Sigma, Cat. No. M6250),

0.2 μM CHIR99021(GSK3i, TOCRIS, Cat. No. 4423),

0.3 μM WH-4-023 (SRC inhibitor, TOCRIS, Cat. No. 5413),

2.5 μM XAV939 (Sigma, Cat. No. X3004) or 2.0 μM IWR-1 (TOCRIS, Cat. No. 3532),

50 μg/ml Vitamin C (Sigma, Cat. No. 49752-100G),

10 ng/ml LIF (Stem Cell Institute, University of Cambridge. SCI),

20 ng/ml ACTIVIN (SCI),

1 ml ITS-X 200× (thermos, 51500056), and

0.3% FBS (Gibco, Cat. No. 10270).

In another embodiment, 500 ml human EPSC media comprises:

482.5 ml DMEM/F-12 (Gibco, Cat. No. 21331-020),

2.5 ml N2 supplement (Thermo Fisher Scientific, Cat. No. 17502048),

5 ml B27 supplement (Thermo Fisher Scientific, Cat. No. 17504044),

5 ml 1× Glutamine Penicillin-Streptomycin (Thermo Fisher Scientific, Cat. No. 11140-050),

5 ml 1×NEAA (Thermo Fisher Scientific, Cat. No. 10378-016),

110 μM 2-mercaptoethanol (Sigma, Cat. No. M6250),

1.0 μM CHIR99021(GSK3 inhibitor, TOCRIS, Cat. No. 4423),

0.1 μM A-419259 (SRC inhibitor, TOCRIS, Cat. No. 3914),

2.5 μM XAV939 (Sigma, Cat. No. X3004) or 2.5 μM IWR-1 (TOCRIS, Cat. No. 3532),

50 μg/ml Vitamin C (Sigma, Cat. No. 49752-100 G), and 10 ng/ml LIF (SCI).

In another embodiment, 500 ml human EPSC media comprises:

F12 DMEM (Gibco, 21331-020), add 240 ml,

Neurobasal medium (Life Technologies, 21103-049) 240 ml,

Penicillin-Streptomycin-Glutamine (100×) (Gibco, 10378016), add 5 ml,

NEAA 100× (Gibco, 11140050), add 5 ml,

Sodium Pyruvate100× (gibco, 11360070), add 5 ml,

14.3M 2-Mercaptoethanol (M6250 Aldrich, Sigma), add 3.8 μl (working concentration 110 μM),

200×N2 (Thermo 17502048), add 2.5 ml,

100×B27 (Thermo 17504044), add 5 ml,

ITS-X 200×(thermos, 51500056), add 2.5 ml,

Vitamin C (Sigma, 49752-100G), working concentration 64 ug/ml,

Bovine Albumin Fraction V (7.5% solution) (Thermo, 15260037), 3 ml,

trace elements B, (Corning, MT99175CI) 1000×

trace elements C, (Corning, MT99176CI) 1000×

reduced glutathione (sigma, G6013-5G) 10 mg/ml, add 165 ul,

defined lipids, (Invitrogen, 11905031) 500×

XAV939 (Sigma X3004), working concentration 2.5 μM,

endo-IWR-1(Tocris, Cat. No. 3532), working concentration 2.5 μM,

A419259 (Tocris Bioscience, 3748), working concentration 0.1 μM,

Chiron 99021 (Tocris Bioscience, 4423), working concentration 1.0 μM, and

Human Lif, working concentration 10 ng/ml.

In one embodiment, 500 ml Porcine EPSC media comprises:

F12 DMEM (Gibco, 21331-020), add 240 ml,

Neurobasal medium (Life Technologies, 21103-049) 240 ml,

Penicillin-Streptomycin-Glutamine (100×) (Gibco, 10378016), add 5 ml,

NEAA 100× (Gibco, 11140050), add 5 ml,

Sodium Pyruvate100× (gibco, 11360070), add 5 ml,

14.3M 2-Mercaptoethanol (M6250 Aldrich, Sigma), add 3.8 μl (working concentration 110 μM),

200×N2 (Thermo 17502048), add 2.5 ml,

100×B27 (Thermo 17504044), add 5 ml,

ITS-X 200×(thermos, 51500056), add 2.5 ml,

Vitamin C (Sigma, 49752-100 G), working concentration 64 ug/ml,

Bovine Albumin Fraction V (7.5% solution) (Thermo, 15260037), 3 ml,

trace elements B, (Corning, MT99175CI) 1000×

trace elements C, (Corning, MT99176CI) 1000×

reduced glutathione (sigma, G6013-5G) 10 mg/ml, add 165 ul,

XAV939 (Sigma X3004), working concentration 2.5 μM,

endo-IWR-1(Tocris, Cat. No. 3532), working concentration 1 μM,

WH-4-023 (Tocris, Cat. No. 5413), working concentration 0.16 μM,

Chiron 99021 (Tocris Bioscience, 4423), working concentration 0.2 μM,

Human Lif, working concentration 10 ng/ml, and

Activin A (STEM CELL TECHNOLOGY, Catalog #78001.1) 20 ng/ml. Suitable chemically defined basal media are described above and include Iscove's Modified Dulbecco's Medium (IMDM), Ham's F12, Advanced Dulbecco's modified eagle medium (DMEM/F12) (Price et al Focus (2003), 25 3-6), RPMI-1640 (Moore, G. E. and Woods L. K., (1976) Tissue Culture Association Manual. 3, 503-508). A preferred chemically defined basal medium is DMEM/F12.

The basal medium may be supplemented by serum-containing or serum-free culture medium supplements and/or additional components. Suitable supplements and additional components are described above and may include L-glutamine or substitutes, such as GlutaMAX-1™, chemically defined lipids, albumin, 1-thiolglycerol, polyvinyl alcohol, insulin, vitamins, such as vitamin C, antibiotics such as penicillin and/or streptomycin and transferrin.

Each of the inhibitors or modulators may be added to the EPSCM to an amount ranging from 0.1 μM to 150 μM; in certain embodiments, in an amount of 0.1 μM, 0.2 μM, 0.3 μM, 0.4 μM, 0.5 μM, 0.6 μM, 0.7 μM, 0.8 μM, 0.9 μM, 1 μM, 1.5 μM, 2 μM, 2.5 μM, 3 μM, 3.5 μM, 4 μM, 4.5 μM, 5 μM, 5.5 μM, 6 μM, 6.5 μM, 7 μM, 7.5 μM, 8 μM, 8.5 μM, 9 μM, 9.5 μM, 10 μM, 11 μM, 12 μM, 13 μM, 14 μM, 15 μM, 16 μM, 17 μM, 18 μM, 19 μM, 20 μM, 25 μM, 30 μM, 35 μM, 40 μM, 45 μM, 50 μM, 60 μM, 70 μM, 80 μM, 90 μM, 100 μM, 110 μM, 120 μM, 130 μM, 140 μM, or 150 μM.

Each of the inhibitors or modulators may be added to the EPSCM to an amount ranging from 0.05 μM to 0.1 μM, 0.1 μM to 1 μM, 1 μM to 2 μM, 2 μM to 3 μM, 3 μM to 4 μM, 4 μM to 5 μM, 5 μM to 6 μM, 6 μM to 7 μM, 7 μM to 8 μM, 8 μM to 9 μM, 9 μM to 10 μM, 10 μM to 15 μM, 15 μM to 20 μM, 20 μM to 30 μM, 30 μM to 40 μM, 40 μM to 50 μM, 50 μM to 60 μM, 60 μM to 70 μM, 70 μM to 80 μM, 80 μM to 90 μM, 90 μM to 100 μM, 100 μM to 110 μM, 110 μM to 120 μM, 120 μM to 130 μM, 130 μM to 140 μM, 140 μM to 150 μM, or 150 μM to 160 μM.

Suitable inhibitors or modulators include natural and synthetic small molecule inhibitors or antibodies. Suitable Mek-ERK, JNK, p38, Src, GSK3 and Wnt pathway inhibitors are known in the art and are commercially available. The Mek-ERK pathway is chain of proteins in the cell that communicates a signal from a receptor on the surface of the cell to the DNA in the nucleus of the cell. The major proteins in this pathway are MEK and ERK. Inhibiting these proteins will disrupt signaling in this pathway. Thus, the inhibitor may directly or indirectly inhibit MEK or ERK such that signaling in this pathway is disrupted. For example, the inhibitor may be a MEK inhibitor or ERK inhibitor.

Suitable Jun N-Terminal Kinase (JNK) inhibitors include JNK Inhibitor VIII (catalogue number sc-202673), RWJ 67657 (catalogue number sc-204251), Antibiotic LL Z1640-2 (catalogue number sc-202055), SX 011 (sc-358841), Bentamapimod (sc-394312), AEG 3482 (sc-202911), from www.scbt.com or SP600125 JNK inhibitor from www.invivogen.com. In one embodiment, the JNK Inhibitor is SP600125.

Suitable p38 inhibitors include sB203580 which inhibits both the a and R isoforms of p38 MAPK available from www.invivogen.com, p38 MAP Kinase Inhibitor IV (catalogue number sc-204159), LY2228820 (catalogue number sc-364525), PH-797804 (catalogue number sc-364579), p38 MAP Kinase Inhibitor (catalogue number sc-204157), SX 011 (sc-358841) and 2-(4-Chlorophenyl)-4-(fluorophenyl)-5-pyridin-4-yl-1,2-dihydropyrazol-3-one (sc-220665) available from www.scbt.com. In one embodiment, the p38 Inhibitor is sB203580.

The Src family kinases (SFK) are a family of non-receptor tyrosine kinases that included nine highly related members. Broad spectrum Src Kinase family inhibitors which inhibit multiple src family members are available and known in the art. Suitable Src Kinase family inhibitors include A-419259 which is a broad spectrum Src family kinase inhibitor (available from Sigma-Aldrich). Other suitable SRK inhibitors include PP1, PP2 and CGP77675 also available from Sigma-Aldrich (www.sigmaaldrich.com), and A419259 trihydrochloride or KB SRC 4 available from Tochris Bioscience (www.tochris.com). In one embodiment, the Src Kinase family inhibitor is WH-4-023 or A-419259.

Suitable GSK3 inhibitors include CHIR99021, a selective and potent GSK3 inhibitor available from Tocris Bioscience(cat 4423), or BIO (cat 3194), A 1070722 (cat 4431), 3F8 (cat 4083), AR-A 014418 (cat 3966), L803-mts (cat 2256) and SB 216763 (cat 1616) also available from Tocris Bioscience(www.tochris.com). Other suitable GSK inhibitors include GSK-3 Inhibitor IX (available from Santa Cruz Biotechnology sc-202634). In one embodiment, the GSK-3 Inhibitor is CHIR99021.

In addition, Wnt inhibitor may be added to the presently disclosed composition. Wnt inhibitor is an antagonist of the Wnt/13-catenin signalling pathway.

The Wnt/13-catenin signaling pathway is the Wnt pathway that causes an accumulation of β-catenin in the cytoplasm and its eventual translocation into the nucleus. In the absence of wnt signaling β-catenin is degraded by a destruction complex which includes the proteins Axin, adenomatosis polyposis coli (APC), protein phosphatase 2A (PP2A), glycogen synthase kinase 3 (GSK3) and casein kinase In (CK1α).

The wnt inhibitor may be a tankyrase inhibitor. Tankyrase inhibition inhibits axin ubiquitinization and stabilises axin protein (Huang et al 2009), therefore inhibiting wnt signalling.

A suitable tankyrase inhibitor is XAV939 (www.sigmaaldrich.com). Additional published tankyrase inhibitors include WIKI4, TC-E 5001 and JW 55, all commercially available from Tocris (www.tocris.com).

An effective amount of an inhibitor may be added to the presently disclosed composition. An effective amount is an amount which is sufficient to inhibit signaling in the pathway or by the protein which is targeted.

The expanded potential stem cell medium (EPSOM) may be a chemically defined medium (CDM).

A chemically defined medium (CDM) is a nutritive solution for culturing cells which contains only specified components, components of known chemical structure in certain embodiments. Therefore, a CDM is devoid of undefined components or constituents which include undefined components, such as feeder cells, stromal cells, serum, matrigel, serum albumin and complex extracellular matrices. Suitable chemically defined basal medium, such as Advanced Dulbecco's modified eagle medium (DMEM) or DMEM/F12 (Price et al Focus (2003) 25 3-6), Iscove's Modified Dulbecco's medium (IMDM) and RPMI-1640 (Moore, G. E. and Woods L. K., (1976) Tissue Culture Association Manual. 3, 503-508; see Table 1), knockout serum replacement (KSR) are known in the art and available from commercial sources (e.g. Sigma-Aldrich MI USA; Life Technologies USA).

In one embodiment, the basal medium is DMEM/F12. The basal medium may comprise or may be supplemented with, AlbuMAX II, which is a commercially available BSA or knockout serum replacement (KSR). The basal medium may also be supplemented with any or all of N2, B27, L-Glutamine, antibiotics (in certain embodiments, Penicillin and Streptomycin); Non-Essential Amino Acids; vitamins (in certain embodiments, vitamin C) and basal medium eagle (bME), all of which are commercially available (for example from Sigma-Aldrich). Other suitable supplements are known in the art and described herein.

In certain embodiments, the following additives may be present in the composition described below

Glutamine, Penicillin and Streptomycin are commercially available as a Penicillin-Glutamine-Streptomycin mix (Cat. No. 11140-050) for example from Thermo Fisher Scientific.

An example of an EPSCM comprises DMEM/F12 basal medium; supplemented with AlbuMAX II or Knockout Serum Replacement and the inhibitors and modulators described herein. The EPSCM may also comprise any of human insulin; N2, B27; Glutamine-Penicillin-Streptomycin; Non-Essential Amino Acids; vitamin C and basal medium eagle (bME), and LIF.

In some embodiments, the population of EPSCs is produced by culturing a population of pluripotent stem cells in the EPSCM for one or more (for example two or more, three or more, four or more, five or more) repeated “passages” to produce a descendent population of EPSCs. Passaging is also referred to as sub-culturing, and is the transfer of cells from a previous culture into fresh growth medium. Cells in a culture follow a characteristic growth pattern of lag phase, log phase and stationary phase. The timings of these phases may vary depending on the cell used (e.g. mammalian cells vs non-mammalian cells). Methods to determine the stage of cell growth are well known in the art. Generally cells are passaged in log phase. In some embodiments the pluripotent stem cells may be passaged (i.e. sub-cultured) one to ten times, three to ten times, three to five times in the EPSCM, to produce the population of EPSCs. In one embodiment, the population is passaged at least three times to produce the population of EPSCs.

EPSCM as described herein may be formulated into a kit for sale.

The one or more culture media in the kit may be formulated in deionized, distilled water. The one or more media will typically be sterilized prior to use to prevent contamination, e.g. by ultraviolet light, heating, irradiation or filtration. The one or more media may be frozen (e.g. at 20° C. or 80° C.) for storage or transport. The one or more media may contain one or more antibiotics to prevent contamination.

The one or more media may be a 1× formulation or a more concentrated formulation, e.g. a 2× to 250× concentrated medium formulation. In a 1× formulation each ingredient in the medium is at the concentration intended for cell culture, for example a concentration set out above. In a concentrated formulation one or more of the ingredients is present at a higher concentration than intended for cell culture. Concentrated culture media are well known in the art, such as salt precipitation or selective filtration. A concentrated medium may be diluted for use with water (in certain embodiments, deionized and distilled) or any appropriate solution, e.g. an aqueous saline solution, an aqueous buffer or a culture medium.

The one or more media in the kit may be contained in hermetically-sealed vessels which prevent contamination. Hermetically-sealed vessels may be preferred for transport or storage of the culture media. The vessel may be any suitable vessel, such as a flask, a plate, a bottle, a jar, a vial or a bag.

The kit may also include instructions for use, e.g. for using the EPSCM to obtain EPSCs.

### 5.3 PFF Reprogramming

Provided herein are repeated PFF reprogramming experiments by directly culturing the primary colonies in pEPSCM (Extended Data FIG. 3e), which generated 11 stable pEPSC^{iPS} lines from 16 primary colonies (70% efficiency). All lines expressed high levels of endogenous pluripotency genes and six of them did not have detectable expression of any of the eight exogenous reprogramming factors (Extended Data FIG. 3f). This pEPSCM condition is subsequently employed to derive stem cell lines directly from porcine preimplantation embryos. A total of 26 lines (pEPSCs^{Emb}, 14 male and 12 female) were established from 76 early blastocysts (5.0 dpc), and 12 cell lines (pEPSCsPar) from 252 parthenogenetic blastocysts (FIG. 1a, Table 1 and Extended Data FIG. 3g). Similar to the pEPSCs^{iPS}, pEPSCs^{Emb} had high nuclear/cytoplasmic ratios, and formed compact colonies with smooth colony edges (FIG. 1a, Extended Data FIG. 3h). The pEPSCs^{Emb} were passaged every 3-4 days at 1:10 ratio as single cells and could be maintained for >40 passages on STO feeders without overt differentiation. Subcloning efficiency was about 10% at low cell density (2,000 cells per well in a 6-well plate), but high cell densities were always used in routine passaging. pEPSCs^{Emb} were karyotypically normal after 25 passages (Extended Data FIG. 4a).

The pEPSCs^{Emb} and pEPSCs^{iPS} expressed pluripotency genes at levels comparable to the blastocysts (Extended Data FIG. 3f), which were verified by immunostaining (Extended Data FIG. 4b). Pluripotency gene expression was drastically reduced or lost when pEPSCs were cultured in one of the seven previously reported porcine ESC media [9-15] (Extended Data FIG. 4c-e). The pEPSCs showed extensive DNA demethylation at the OCT4 and NANOG promoter regions (FIG. 1b), and had OCT4 distal enhancer activity (Extended Data FIG. 4f). The EPSCs were amenable for Crispr/Cas9-mediated insertion of an H2B-mCherry expression cassette into the ROSA26 locus (Extended Data FIGS. 4g and 4h). In vitro, pEPSCs differentiated to tissues expressing genes representative of the three germ layers: SOX7, AFP, T, DES, CRABP2, SMA, β-Tubulin and PAX6 and, uniquely, the trophoblast genes HAND1, GATA3, PGF, KRT7, ELF4, KRT8, ITGB4, TEAD3, TEAD4, SDC1 and PLET1 (FIG. 1c, Extended Data FIG. 4i). In immunocompromised mice, pEPSCs^{Emb} formed mature teratomas with derivatives of the three germ layers, even including placental lactogen-1 (PL1), KRT7- and SDC1-positive trophoblast-like cells (FIG. 1d-1e and Extended FIG. 4j). These results indicate that pEPSCs^{Emb} and pEPSCs^{iPS}, like mEPSCs [1], may possess an expanded developmental potential for both the embryonic cell lineages and extra-embryonic trophoblast lineages. The pEPSCs were tested for their contribution to blastocyst cell lineages in chimeras. Following incorporation of the pEPSCs into preimplantation embryos and after 48 hours of culture, pEPSCs (marked by EF1a-H2B-mCherry) had colonized both the trophectoderm and inner cell mass of blastocysts(Extended Data FIG. 5a). Following transfer of the chimeric embryos to synchronized recipient sows, a total of 45 conceptuses were harvested from 3 litters at days 26-28 of gestation (Supplementary Table 2, Extended Data FIG. 5b). Flow cytometry of dissociated cells from embryonic and extraembryonic tissues of the chimeras revealed the presence of mCherry⁺ cells in 7 conceptuses (Extended Data FIG. 5c, Supplementary Table 3 and Table 4): mCherry⁺ cells in both the placenta and embryonic tissues in 2 chimeras (#8 and #16); only in embryonic tissues in 3 chimeras (#4, #21 and #34); and exclusively in the placenta of 2 chimeras (#3 and #6). Genomic DNA PCR assays detected mCherry DNA only in those seven mCherry⁺ chimeras, but not in any other conceptuses (Extended Data FIG. 5d, Supplementary Table 3 and 4). Despite the overall low contributions from the donor mCherry⁺ cells, they were found in multiple host embryonic tissues and organs that were identified by the following tissue lineage markers: SOX2, TUJ1, GATA4, SOX17, AFP, α-SMA, PL-1 and KRT7 (FIG. 1f-g and Extended Data FIG. 5e-f).

### 5.4 PGC Testing

pEPSCs are tested to see if they had the potential to produce PGC-like cells (PGCLCs) in vitro, similar to mouse and human pluripotent stem cells [25-27]. In early-primitive streak (PS)-stage porcine embryos (E11.5E12), the first cluster of porcine PGCs can be detected as SOX17⁺ cells in the posterior end of the nascent primitive streak, and these cells later co-express OCT4, NANOG, BLIMP1 and TFAP2C [26]. NANOS3 is an evolutionarily conserved PGC-specific factor [28, 29] and human NANOS3 reporter cells have been used for studying the derivation of PGCLCs from pluripotent stem cells [26, 27]. To facilitate identification of putative porcine PGCLCs, the H2BmCherry reporter cassette are targeted to the 3′ UTR of the NANOS3 locus in pEPSCs^{Emb} (Line K3, male) (Extended Data FIG. 6a). After expressing the SOX17 transgene transiently for 12 hours, the pEPSCs^{Emb} harboring the NANOS3 reporter were allowed to form embryoid bodies (EBs) (Extended Data FIG. 6b), which contained cell clusters co-expressing NANOS3 (mCherry⁺) and tissue-nonspecific alkaline phosphatase (TNAP, a PGC marker) within 3-4 days (FIG. 2a).

The derivation of putative porcine PGCLCs was BMP2/4 dependent, as removal of BMP2 from the EB culture or inhibition of the BMP2/4 signaling by inhibitor LDN-193189 abrogated the formation of mCherry⁺/TNAP⁺ cell clusters (FIG. 2a). Expressing NANOG, BLIMP1 or TFAP2C transgenes in pEPSCs, either individually or in combinations, had no effect on the preponderance of NANOS3⁺ cells (Extended Data FIG. 6c), which was different from the reported derivation of human PGCLCs [26]. However, co-expression of SOX/7 with BLIMP1, but not NANOG or TFAP2C, appeared to increase the population of NANOS3⁺ cells (Extended Data FIGS. 6c and 6c).

The mCherry⁺ (NANOS3⁺) putative PGCLCs within the EBs expressed PGC-specific genes NANOS3, BLIMP1, TFAP2C, CD38, DND1, KIT and OCT4 [33], which were detected in RT-qPCR and was confirmed by immunofluorescence at single cell resolution (FIG. 2b-c, and Extended Data FIG. 6e). Specific RNA-seq analysis of the mCherry⁺/NANOS3⁺ cells revealed expression of early PGC genes (OCT4, NANOG, LIN28A, TFAP2C, CD38, DND1, NANOS3, ITGB3, SOX15 and KIT), and reduced SOX2 expression (FIG. 2d-e, Supplementary Table 5) [27]. During PGCLC derivation from human ESCs, cells undergo global DNA demethylation, which is accompanied by upregulation of TETs and down-regulation of DNMT3A/B [27]. Similarly, relative to the parental pEPSCs^{Emb}, DNMT3B was down-regulated in porcine mCherry⁺/NANOS3⁺ cells, whereas TET1/2 were up-regulated (FIG. 2e-f, Supplementary Table 5).

### 5.5 In Vitro Culture of Human ES Cell

Human ESCs have been widely used in studying human embryo development in vitro and hold great potential for regenerative medicine. [36-37] The finding that inhibition of SRC and Tankyrases is sufficient to convert mouse ESCs to mEPSCs [1] and that these two inhibitors are required for the generation of pEPSCs raises the possibility that similar in vitro culture conditions may also work for other mammalian species. To explore this possibility, four established human ES cell (hESC) lines (H1, H9, Man1 or M1, and Man10 or M10 cells) [30-32] are cultured in pEPSCM and passaged them up to three times. The cells displayed diverse morphologies and heterogeneous expression of OCT4 (Extended Data FIG. 7a). Removing ACTIVIN A (20 ng/ml) from pEPSCM led to considerably fewer cell colonies formed from H1 (<1.0%) and M1 (5.0%) ESC cultures, while none from H9 or M10 (Extended Data FIG. 7a), which is consistent with the inherent between-line heterogeneity of human ESCs [33, 34]. With further refinement of the culture conditions (for example, replacing WH-4-023 with another SRC inhibitor A419259 in hEPSCM, see Methods), morphologically homogenous and stable cell lines were established from single-cell sub-cloned H1 (H1-EPSCs) and M1 cells (M1-EPSCs) (FIG. 3a). Karyotype analysis of H1 and M1 cells grown in hEPSCM on STO feeders revealed genetic stability (at passage 25 post conversion from the parental hESCs, Extended Data FIG. 7b).

When human primary iPSC colonies reprogrammed from dermal fibroblasts were directly cultured in hEPSCM, around 70% of the picked colonies could be established as stable iPSC lines (iPSC-EPSCs) (Extended Data FIG. 7c). These iPSCs expressed pluripotency markers with no obvious leakiness of the exogenous reprogramming factors (Extended Data FIG. 7d-e). The H1-EPSCs proliferated more robustly than the H1 ESCs cultured in standard FGF-containing medium (H1-ESC, primed) or under naïve 5i/L/A conditions (H1-naïve ESC) [22] (Extended Data FIG. 7f), and were tolerant of single cell passaging with about 10% single cell sub-cloning efficiency in the transient presence of ROCKi. Cell survival at passaging was substantially improved in the presence of 5.0 ng/ml ACTIVIN A or by splitting the cells at higher density. Human EPSCs expressed pluripotency genes (OCT4, SOX2, NANOG, REX1 and SALL4) at higher levels than the H1-ESCs (Extended Data FIG. 7d) and minimal levels of lineage markers (EOMES, GATA4, GATA6, T, SOX17 and RUNX1) (Extended Data FIG. 7g). Expression of core pluripotency factors and surface markers in human EPSCs was confirmed by immunostaining (Extended Data FIG. 7h). H1EPSCs differentiated to derivatives of the three germ layers in vitro and in vivo (Extended Data FIG. 7i-j). Moreover, H1-EPSCs were successfully differentiated to PGCLCs using in vitro conditions developed for germ cell competent hESCs or iPSCs [26, 27] (Extended Data FIG. 7k-l).

These results demonstrate that human and porcine EPSCs could be derived and maintained using the similar set of small molecule inhibitors. Global gene expression profiling revealed that pEPSCs and hEPSCs were clustered together, and were distinct from PFFs or other human pluripotent stem cells [1, 42, 43] (FIG. 3b, Extended Data FIG. 8a and Supplementary Table 6-7). Both porcine and human EPSCs expressed high levels of key pluripotency genes, low levels of somatic cell lineage genes, PAX6, T, GATA4 and SOX7, or placenta-related genes such as PGF, TFAP2C, EGFR, SDC1 and ITGA5 (Extended Data FIG. 8b-e). Consistent with the high level of global DNA methylation of pEPSCs and hEPSCs (Extended Data FIG. 9a), DNA methyltransferase genes DNMT1 and DNMT3A and DNMT3B were highly expressed, whereas TET1, TET2 and TET3 were expressed at lower levels (Extended Data FIG. 9b-c). Among the highly expressed 76 genes (>8-fold increase) in H1-EPSC in comparison to H1-ESCs, 17 genes encode histone variants with 15 belonging to the histone cluster 1 (FIG. 3c and Supplementary Table 8). Interestingly, these histone genes were expressed at low levels in 5i and primed human ESCs but were highly expressed in human 8-cell and morula stage embryos (FIG. 3d). The significantly higher expression of these histone genes was further confirmed in more hEPSC lines when compared with the same cells cultured either in conventional human ESC medium (FGF) or 5i (naïve) medium (FIG. 3e).

The biological significance of the high histone gene expression in hEPSCs and in human 8-cell and morula stage embryos remains to be further investigated. Single cell RNA-seq (scRNAseq) of porcine and human EPSCs revealed uniform expression of the core pluripotency factors: OCT4, SOX2, NANOG and SALL4 (FIG. 3f), and substantially homogenous cell cultures (FIG. 3g). At the single-cell level, mouse EPSCs had enriched transcriptomic features of 4-cell to 8-cell blastomeres [1]. The scRNAseq analysis of hEPSCs indicated that they were transcriptionally more similar to human 8-cell to morula stage embryos [44, 45] as compared with other stages of human preimplantation embryos (FIG. 3h, and Extended Data FIG. 8f), and in line with the histone gene expression profiles in RT-qPCR, bulk RNAseq and scRNAseq (FIG. 3d and Extended Data FIG. 9e). Interestingly, transcriptome analysis also revealed low expression of naïve pluripotency factors such as KLF2 in EPSCs (FIG. 3f and Extended Data FIG. 8b-c), which are not expressed in human early preimplantation embryos. [46] Although KLF2, TET1, TET2 and TET3 were weakly expressed in both pEPSCs and hEPSCs (Extended Data FIG. 8b and Extended Data FIG. 9b, 9c), their promoter regions were characterized by active H3K4m3 histone marks (Extended Data FIG. 9f). In contrast to pluripotency genes, the cell lineage gene loci (e.g. CDX2, GATA2, GATA4, SOX7 and PDX1) had high H3K27me3 and low H3K4me3 marks, respectively, in both porcine and human EPSCs (Extended Data FIG. 9f).

### 5.6 Signal Pathways

hEPSCs and pEPSCs shared similar signalling requirements as revealed by the impacts after removal of individual components from the culture medium. Removal of the SRC inhibitor WH-4-023 or A419259 reduced expression of pluripotency factors in both EPSCs (Extended Data FIG. 10a-d). Notably, in human EPSCs, using the SRC inhibitor WH-4-023 instead of A419259 led to lower pluripotency gene expression (Extended Data FIG. 10b). Similar to mEPSCs, [1] XAV939 enhanced AXIN1 protein content (Extended Data FIG. 10e), and reduced canonical WNT activities in both EPSCs (Extended Data FIG. 10f). Withdrawal of XAV939 caused collapse and differentiation of these EPSCs (Extended Data FIGS. 10a-b, 10d, and 10g-k). SMAD2/3 were phosphorylated in EPSCs (Extended Data FIG. 10e). Either removing ACTIVIN A from pEPSCM or adding the TGFβ inhibitor SB431542 resulted in massive cell loss and down-regulation of pluripotency factors in pEPSCs (Extended Data FIGS. 10a, 10g, 10h and 10j), whereas in human EPSCs, the TGFβ inhibitor SB431542 induced rapid cell differentiation with preferential expression of trophoblast lineage transcription factor genes CDX2, ELF5 and GATA2 (Extended Data FIGS. 10b, 10i and 10k). At a relatively low concentration of exogenous ACTIVIN A (5.0 ng/ml), hEPSCs showed a stronger propensity for embryonic mesendoderm lineage differentiation (Extended Data FIG. 10l), and generated more NANOS3-tdTomato⁺ PGCLCs (Extended Data FIG. 10m-n). Removing CHIR99021 and Vitamin C from pEPSCM did not affect pluripotency gene expression but reduced the number of colonies from single cells (Extended Data FIGS. 10a and 10h), whereas a high CHIR99021 concentration (3.0 μM) induced differentiation of both porcine and human EPSCs (Extended Data FIGS. 10a, 10h and 10j), similar to that in human or rat naïve cells. [30, 47] INK and BRAF inhibition might improve culture efficiency, but was not essential (Extended Data FIG. 10h-i). In hEPSCs, the requirements for CHIR99021 and Vc were similar to pEPSCs (Extended Data FIGS. 10a-b and 10h-I). Derivation of mouse naïve ESCs required 1.0 □M Mek1/2 inhibitor PD0325901 [26], but this concentration of PD0325901 was deleterious to porcine cells in the screens for pEPSC culture conditions (Extended Data FIG. 2b-2f). Consistent with this observation, even 0.1 μM PD0325901 decreased pEPSC survival as measured by colony formation in serial passaging (Extended Data 10 h). The full details of porcine and human EPSC culture conditions are included in Methods.

### 5.7 Differentiation

The differentiation of hEPSCs to trophoblast cells was tracked by expression of CDX2-Venus reporter (T2A-Venus inserted into the 3′ UTR of the CDX2 locus) (Extended Data FIG. 11a). Inhibiting TGFβ by SB431542 resulted in 70% of the CDX2 reporter cells being CDX2-Venus⁺ (FIG. 4a), whereas essentially no CDX2-Venus⁺ cells were detected if the reporter cells were cultured in FGF or under the 5i naïve ESC conditions. Expression of trophoblast related genes such as CDX2, GATA3, ELF5, KRT7, TFAP2C, PGF, HAND1 and CGA was rapidly increased in differentiating H1-EPSCs and iPSC-EPSCs but not in H1-ESCs or H1-5i naïve cells (FIG. 4b). Addition of BMP4, which promotes differentiation of human ESCs to putative trophoblasts, [48] induced expression of trophoblast genes at a much higher level in H1-EPSCs and iPSC-EPSCs than in H1-ESCs or H1-5i naïve ESCs (Extended Data FIG. 11b). Inhibiting FGF and TGFβ signalling while in parallel activating BMP4 was reported to effectively induce trophoblast differentiation in FGF-cultured (primed) human ESCs. [49-50] Under these conditions, expression of trophoblast genes, especially the late trophoblast genes GCM1, CGA and CGB, was still much higher in H1-EPSCs than in H1-ESCs, whereas naïve 5i hESCs displayed no trophoblast differentiation (Extended Data FIG. 11c). Global gene expression analysis demonstrated that under TGFβ signalling inhibition H1-EPSCs and iPSC-EPSCs followed a differentiation trajectory distinct from the H1-ESCs (FIG. 4c), and that in cells differentiated from EPSCs, but not from H1-ESCs, important trophoblast development or function genes were highly expressed including: (1) BMP4 on days 2-4 of differentiation; (2) genes of human endogenous retrovirus-encoded envelope protein Syncytin-1 (ERVW-1) and Syncytin-2 (ERVFRD-1) that promote cytotrophoblast fusion into syncytiotrophoblast; (3) the maternally expressed gene p57 (encoded by CDKN1C) which is expressed in trophoblast cells and is essential for normal placenta development [51-52]; (4) CD274 (encoding PD-L1 or B7-H1) that modulates immune cell activities; and (5) EGFR which is important in human trophoblast stem cells (hTSCs)⁵³ (Extended Data FIG. 11d and Supplementary Table 6).

To further infer the identity of the differentiated hEPSCs by TGFβ inhibition, we performed Pearson correlation coefficient analysis of the transcriptome of cells differentiated from H1-EPSCs, iPSC-EPSCs or H1-ESCs with external reference data including primary human trophoblasts (PHTs) and human placenta tissues, [50] which again revealed the similarity between cells differentiated from hEPSCs and PHTs and the placenta (Extended Data FIG. 11e). The cells differentiated from H1-EPSCs by TGFβ inhibition expressed human trophoblast specific miRNAs (C19MC miRNAs: hsa-miR-525-3p, hsa-miR-526b-3p, hsa-miR-517-5p, and hsa-miR-517b-3p) [54] (Extended Data FIG. 11f-g), displayed DNA demethylation at the ELF5 locus [55, 56] (Extended Data FIG. 11h), and produced abundant amounts of placental hormones (Extended Data FIG. 11i-j).

When hEPSCs (ESC-converted-EPSCs and iPSC-EPSCs) were cultured in human trophoblast stem cell (hTSC) conditions [53] with low cell density (2,000 cells/3.5 cm dish), colonies with TSC morphology formed after 7-9 days (FIG. 4d). These colonies were picked and expanded into stable cell lines under hTSC conditions with up to 30% line establishment efficiency (FIG. 4d). On the other hand, hTSC lines were not established from human H1 or M1 ESCs, whether they were cultured under primed or naïve ESCs conditions. The hEPSC-derived TSC-like cells (referred in this study as hTSCs) expressed trophoblast transcription regulators: GATA2, GATA3 and TFAP2C but had down-regulated pluripotency genes (FIG. 4e and Extended Data FIG. 12a). Compared to gene expression changes during human EPSCs differentiation to trophoblasts, hTSCs derived from hEPSCs had enriched transcriptomic features of day 4-6 differentiated human EPSCs under TGFβ inhibition (Extended Data FIG. 12b). Following the published protocols, [53] hTSCs were differentiated to both multinucleated syncytiotrophoblasts (ST) and HLA-G⁺ extravillous trophoblasts (EVT) (FIG. 4f-4g, and Extended Data FIG. 12c-12h). Once injected into immunocompromised mice, hTSCs formed lesions which contained cells positively stained for trophoblast markers SDC1 and KRT7 (FIG. 4h, and Extended Data FIG. 12i). Additionally, high levels of hCG (human chorionic gonadotropin) were detected in blood of the mice forming lesions from injected hTSCs but not in mice injected with vehicle controls (Extended Data FIG. 12j). Although both porcine and human EPSCs did not express high levels of placenta development-related genes such as PGF, TFAP2C, EGFR, SDC1 and ITGA5 (Extended Data FIG. 8d-e), both cells had high H3K4me3 at these loci (Extended Data FIG. 13a), clearly underpinning EPSCs' trophoblast potency. In line with the molecular similarities between human and porcine EPSCs, under human TSC conditions, stable TSC-like lines could also be derived from porcine EPSCs^{Emb} (referred here as pTSCs. Extended Data FIG. 13b). pTSCs expressed trophoblast genes, formed lesions which contained cells positively stained for SDC1 and KRT7 in immunocompromised mice (Extended Data FIG. 13c-13f). When introduced into porcine preimplantation embryos, descendants of pTSCs were localised in the trophectoderm and expressed GATA3 (Extended Data FIG. 13g). These results therefore provide compelling evidence that human and porcine EPSCs possessed expanded differentiation potential that encompasses the trophoblast lineage.

One of the key mechanisms for derivation and maintenance of EPSCs of mouse, porcine and human is blocking poly(ADP-ribosyl)ation activities of PARP family members TNKS1/2 using small molecule inhibitors such as XAV939. [57, 58] In human cells, poly(ADP-ribose) in proteins is removed by poly(ADP-ribose) glycohydrolase (PARG) and ADP-ribosylhydrolase 3 (ARH3). [59] Genetic inactivation of Parp1/2 and TIVKS1/2 in the mouse caused trophoblast phenotypes, [60] whereas inactivating Parg led to loss of functional trophectoderm and TSCs. [61] PARG is tested whether it was of any relevance to hEPSCs developmental potential to derive trophoblasts. In hEPSCs, PARG-deficiency did not appear to cause noticeable changes in EPSC culture but adversely affected trophoblast differentiation (Extended Data FIG. 14a-d), which may indicate an evolutionally conserved mechanism for EPSCs and trophoblast development from mouse to human.

The present subject matter described herein will be illustrated more specifically by the following non-limiting examples, it being understood that changes and the variations can be made therein without deviating from the scope and the spirit of the disclosure as hereinafter claimed. It is also understood that various theories as to why the disclosure works are not intended to be limiting.

### 6. EXAMPLES

### 6.1 Ethical Considerations of Working with Human ESCs

The experiments of using human ESCs and human cells were approved by HMDMC of the Wellcome Trust Sanger Institute, Cambridge UK. The experiments using porcine embryos were approved by the Niedersaechsisches Landesamt fuer Verbraucherschutz and Lebensmittelsicherheit, LAVES, Oldenburg Germany. The mouse teratoma Experiments were performed in accordance with UK Home Office regulations and the Animals (Scientific Procedures) Act 1986 (license number 80/2552), and were approved by the Animal Welfare and Ethical Review Body of the Wellcome Genome Campus, and the Committee on the Use of Live Animals in Teaching and Research, The University of Hong Kong (CULATR, HKU). At the end of the study, mice were euthanized by cervical dislocation, in accordance with stated UK Home Office regulations

### 6.2 Culturing Porcine and Human EPSCs

Porcine and human EPSC cultures were routinely maintained on STO feeders. STO feeder plates were prepared 3-4 days before passaging by thawing and plating the mitomycin C inactivated STO cells on 0.1% gelatinised plates at the density of ˜1.1×10⁴ cells/cm². Porcine/human EPSC cells were maintained on STO feeder layers and enzymatically passaged every 3-5 days by a brief PBS wash followed by treatment for 3-5 minutes with 0.25% trypsin/EDTA (Gibco, Cat. No. 25500-054). The cells were dissociated and centrifuged (300 g×5 minutes) in M10 medium. M10: knockout DMEM (Gibco, Cat. No. 10829-018), 10% FBS (Gibco, Cat. No. 10270), 1× Glutamine Penicillin-Streptomycin (Thermo Fisher Scientific, Cat. No. 11140050) and 1×NEAA (Thermo Fisher Scientific, Cat. No. 10378-016). After removing supernatant, the porcine/human EPSCs were re-suspended and seeded in pEPSCM/hEPSCM supplemented with 5 μM ROCK inhibitor Y-27632 (Tocris, Cat. No. 1254). 5% FBS (Gibco, Cat. No. 10270) and 10% KnockOut Serum Replacement (KSR) (Gibco, Cat. No. 10828028) were added in pEPSCM and hEPSCM respectively to improve cells survive. 12-24 hours later, medium was switched to pEPSCM/hEPSCM only. Both pEPSCM and hEPSCM are N2B27 based media. N2B27 basal media (500 ml) was prepared by inclusion of the following components: 482.5 ml DMEM/F-12 (Gibco, Cat. No. 21331-020), 2.5 ml N2 supplement (Thermo Fisher Scientific, Cat. No. 17502048), 5 ml B27 supplement (Thermo Fisher Scientific, Cat. No. 17504044), 5 ml 1× Glutamine Penicillin-Streptomycin (Thermo Fisher Scientific, Cat. No. 11140-050), and 5 ml 1×NEAA (Thermo Fisher Scientific, Cat. No. 10378-016), 0.1 mM 2-mercaptoethanol (Sigma, Cat. No. M6250). pEPSCM (500 ml) was generated by adding the following small molecules and cytokines into 500 ml N2B27 basal media: 0.2 μM CHIR99021(GSK3i, TOCRIS, Cat. No. 4423), 1 μM WH-4-023 (SRC inhibitor, TOCRIS, Cat. No. 5413), 2.5 μM XAV939 (Sigma, Cat. No. X3004) or 2.5 μM IWR-1 (TOCRIS, Cat. No. 3532), 50 ng/ml Vitamin C (Sigma, Cat. No. 49752-100G), 10 ng/ml LIF (Stem Cell Institute, University of Cambridge. SCI) and 20 ng/ml ACTIVIN (SCI). hEPSCM (500 ml) was generated by adding the following components into 500 ml N2B27 basal media: 1.0 μM CHIR99021(GSK3 inhibitor, TOCRIS, Cat. No. 4423), 0.5 μM A-419259 (SRC inhibitor, TOCRIS, Cat. No. 3914), 2.5 μM XAV939 (Sigma, Cat. No. X3004), 50 ng/ml Vitamin C (Sigma, Cat. No. 49752-100G), 10 ng/ml LIF (SCI). Although both targeting SRC family kinases (SFKs), WH-4-023 and A419259 were preferred for porcine and human EPSCs, respectively. Both porcine and human EPSCs need CHIR99021 for improved proliferation. The high concentration of CHIR99021 (e.g. 3.0 μM) used for mouse ES cells culture induces porcine and human EPSC differentiation. The concentrations of CHIR99021 for porcine and human EPSC cultures are 0.2 μM and 1.0 μM, respectively. The human EPSC culture condition does not contain 0.3% FBS. 0.25 μM SB 590885 (BRAF inhibitor, R&D, Cat. No. 2650) and 2.0 μM SP600125 (INK inhibitor, TOCRIS, Cat. No. 1496) were included to improve porcine and human EPSC cultures, but they were not essential for the routine maintenance of porcine and human EPSCs. All cell cultures in this paper were performed under conditions of 37° C. and 5% CO₂ unless stated otherwise.

### 6.3 Reprogramming PFFs (Porcine Fetal Fibroblasts) to iPSCs

Germany Landrace [1] and China TAIHU OCT4-TD-tomato [2] Porcine fetal fibroblasts (PFFs) were plated on gelatinized 15-cm tissue culture plates and cultured in M20 media. They were trypsinized with 0.25% trypsin/EDTA solution (Gibco, Cat. No. 25500-054) and harvested for electroporation at 80% confluence. M20: knockout DMEM (Gibco, Cat. No. 10829-018), 20% FBS (Gibco, Cat. No. 10270), 1× Glutamine Penicillin-Streptomycin (Thermo Fisher Scientific, Cat. No. 11140-050) and 1×NEAA (Thermo Fisher Scientific, Cat. No. 10378-016). The transfections were performed using an Amaxa Nucleofector machine (Lonza) according to the manufacturer's protocol (NHDF Nucleofector® Kit, Cat. No. VPD-1001, program U-20). piggyBac transposition was used to achieve stable integration of reprogramming factors. The expression of the reprogramming factors was under the transcriptional control of the tetO2 tetracycline/doxycycline inducible promoter. 1.5 million PFFs and 6.0 μg DNA (2.0 μg PB-TRE-pOSCK, Porcine OCT4, SOX2, cMYC and KLF4; 1.0 μg PB-TRE-pNhL, 1.0 μg PB-TRE-hRL: human RARG and TRH1, 1.0 μg PB-EF1a-transposase and 1.0 μg PB-EF1a-rTTA) were used in each electroporation reaction. PB-TRE-pOSCK: cDNAs of porcine OCT4, SOX2, cMYC and KLF4 linked by 2A sequence were expressed as a single transcript [3] from the tetO2 promoter. PB-TRE-pNhL contains cDNAs of porcine NANOG and human LIN28, also linked with 2A sequence [3]. PB-TRE-RL has 2A linked human RARG and TRH1 cDNAs [4]. EF1a promoter was employed to drive the PB transposase expression. Reverse tetracycline controlled transactivator (rtTA) was expressed to induce the expression of the reprogramming factors upon Dox addition. After transfection, 0.2 million PFFs were seeded on mitomycininactivated STO feeders in M15 supplemented with LIF (10 ng/ml, SCI) and Vitamin C (Sigma, Cat. No. 49752-100G) in 10-cm dishes. M15: knockout DMEM (Gibco, Cat. No. 10829-018), 15% FBS (Gibco, Cat. No. 10270), 1× Glutamine Penicillin-Streptomycin (Thermo Fisher Scientific, Cat. No. 11140-050), 1×NEAA (Thermo Fisher Scientific, Cat. No. 10378-016) and 0.1 mM 2-mercaptoethanol (Sigma, Cat. No. M6250). Doxycycline (Dox) (1.0 μg/mL, Sigma, Cat. No. D9891) was added for induction of reprogramming factor expression. The culture media was changed each other day. For transgene dependent iPSC generation, the colonies were picked in M15 at day 12 supplemented with Dox, 50 μg/ml Vitamin C and 10 ng/ml bFGF (SCI) and maintained in the same media. For directly establishing transgene independent iPSCs lines in pEPSCM, Dox was removed at day 9 and the media was switch to pEPSCM immediately. The Dox independent iPSCs colonies were picked in pEPSCM supplemented with 5μM ROCK inhibitor Y27632 (Tocris, Cat. No. 1254) on day 14-15. Y26537 was removed from the culture media 24 hours later and pEPSCM was refreshed every day subsequently.

### 6.4 Screening for the Porcine EPSC Culture Conditions

Dox dependent porcine iPSCs were dissociated in 0.25% trypsin/EDTA solution (Gibco, Cat. No. 25500-054) and seeded in 24-well STO feeder plates at a density of 1×10⁴ cells per well. The cells were cultured in M15 supplemented with Dox (Sigma, Cat. No. D9891), Vitamin C (Sigma, Cat. No. 49752-100G) and 10 ng/ml bFGF (SCI) for two days before the culture media was switched to medium supplemented with indicated small molecules and cytokines (Supplementary Table 1). M15 and N2B27 media: see above. AlbumMax media: DMEM/F12 (Gibco, Cat. No. 21331-020), 20% AlbumMax II (Gibco, Cat. No. 11021-037), 25 mg/mL Human Insulin (Sigma, Cat. No. 91077C), 2×B27 Supplement, 100 ug/mL IGFII (R&D, Cat. No. 292-G2-250), 1×Glutamine Penicillin-Streptomycin (Thermo Fisher Scientific, Cat. No. 11140-050), 1×NEAA (Thermo Fisher Scientific, Cat. No. 10378-016) and 0.1 mM 2mercaptoethanol (Sigma, Cat. No. M6250). 20% KSR media: DMEM/F-12 (Gibco, Cat. No. 21331-020), 20% KnockOut Serum Replacement (KSR) (Gibco, Cat. No. 10828-028), 1× glutamine penicillin-streptomycin (Thermo Fisher Scientific, Cat. No. 11140-050), 1×NEAA (Thermo Fisher Scientific, Cat. No. 10378-016) and 0.1 mM 2-mercaptoethanol (Sigma, Cat. No. M6250). Small molecules and cytokines were supplemented as indicated at the following final concentrations: CHIR99021 (0.2 or 3 μM, TOCRIS, Cat. No. 4423), PD0325901 (0.1 μM and 1 μM, TOCRIS, Cat. No. 22854192); WH-4-023 (4 μM, TOCRIS, Cat. No. 5413), PKC inhibitor Go6983 (5 μM. TOCRIS, Cat. No. 2285); SB203580 (p38 inhibitor, 10 μM. TOCRIS, Cat. No. 1202); SP600125 (JNK inhibitor, 4 μM. TOCRIS, Cat. No. 1496); Vitamin C (50 μg/ml. Sigma, Cat. No. 49752-100G), SB590885 (BRAF inhibitor, 0.25 μM, R&D, Cat. No. 2650), XAV939 (2.5 μM, Cat. No. X3004), R04929097 (Notch signaling inhibitor, 10 μM, Selleckchem, Cat. No. S1575), LDN193189 (BMP inhibitor, 0.1 μM, Sigma, Cat. No. SML0559), Y27632 (ROCKi, 5 μM, Tocris, Cat. No. 1254), Verteporfin (YAP inhibitor, 10 μM, Tocris, Cat. No. 5305). LIF (10 ng/ml, SCI), BMP4 (10 ng/ml, R&D, Cat. No. 5020-BP), SCF (50 ng/ml, R&D, Cat. No. 255-SC-010), EGF (50 ng/ml, R&D, Cat. No. 236-EG-200), TGFβ (10 ng/ml, Cat. No. 7754-BH-005), bFGF (10 ng/ml, SCI), ACTIVIN (20 ng/ml, SCI). The medium was refreshed every day and the surviving cells were passaged at day 6. In the first 24 hours after passaging, 5 uM of ROCKi Y27632 (Tocris, Cat. No. 1254) was supplemented in the media and removed 24 hours later. After 4 days of growing, the colonies survived were collected for RT-qPCR analysis to check the endogenous porcine OCT4 and NANOG expression.

### 6.5 Sow Superovulation

Peripubertal German Landrace gilts (approx. 7-9 months of age, 90-120 kg bodyweight) served as embryo donors. Gilts were synchronized by feeding 5 ml/day/gilt altrenogest (Regumate®, 4 mg/ml, MSD Animal Health, Germany) for 13 days. Followed by an injection of 1500 IU PMSG (Intergonan® 240 I.E./ml, MSD Animal Health, Germany) on the last day of Altrenogest feeding [5]. Ovulation was induced by intramuscular injection of 500 IU of hCG (Ovogest® 300 I.E./ml, MSD Animal Health, Germany) 76 hours later.

### 6.6 Sows Insemination and Embryo Recovery

Semen was collected from Germany Landrace boars [1] via the hand-gloved method using phantom and was immediately diluted in Androhep□Plus solution (Minitube, Tiefenbach, Germany). The sows were artificially inseminated twice at 40 hours and 48 hours, after hCG administration. Five days after the second insemination, sows were slaughtered and the uterus was excised and flushed with Dulbecco's PBS medium (AppliChem, Cat. No. A0964) supplemented with 1% Newborn Calf Serum (NBCS, Gibco™, Cat. No. 16010159). Collected morulae were either directly used for injection experiments or cultured overnight in PZM-3 medium to blastocyst stage and used for ICM isolation (PZM-3 medium: 108 mM Sodium chloride (NaCl, Sigma-Aldrich, Cat. No. S5886), 10 mM Potassium chloride (KCl, Sigma-Aldrich, P-5405), 0.35 mM Potassium phosphate monobasic (KH₂PO₄, SigmaAldrich, Cat. No. P5655), 0.40 mM Magnesium Sulfate heptahydrate (MgSO₄×7 H₂O, Sigma-Aldrich, Cat. No. M5921), 25.07 mM Sodium bicarbonate (NaHCO₃, Sigma-Aldrich, S4019), 2 mM L(+) Lactic acid calcium salt pentahydrate (C₆H₁₀CaO₆×5 H₂O, Roth, Cat. No. 4071), 0.2 mM Sodium pyruvate (Sigma-Aldrich, Cat. No. P2256), 1 mM L-Glutamine (AppliChem, Cat. No. A3704), 0.05 mg/ml Gentamicin sulfate salt (Sigma-Aldrich, Cat. No. G3632), 0.55 mg/ml Hypotaurine (Sigma-Aldrich, Cat. No. H1384), 20 μl/ml BME amino acids solution (Sigma-Aldrich, Cat. No. B6766), 10 μl/ml MEM Non-essential Amino Acid Solution (Sigma-Aldrich, Cat. No. M7145) and 3 mg/ml Bovine Serum Albumin (BSA, Sigma-Aldrich, A7030)).

### 6.7 Oocyte Collection, In Vitro Maturation (IVM) and Generation of Parthenogenetic Embryos

Porcine ovaries from prepubertal gilts were transported at 30° C. from a local abattoir and washed three times with 0.9% Sodium Chloride (NaCl, Sigma-Aldrich, Cat. No. S5886) containing 0.06 mg/ml Penicilin G potassium salt (AppliChem, Cat. No. A1837) and 0.131 mg/ml Streptomycin sulfate (AppliChem, Cat. No. A1852). Oocytes were aspirated from follicles with a diameter of 2-6 mm using an 18-gauge needle and washed in Dulbecco's PBS medium (AppliChem, Cat. No. A0964) supplemented with 0.33 mM Sodium Pyruvate (Sigma-Aldrich, Cat. No. P2256), 5.56 mM D(+)-Glucose Monohydrate (Roth, Cat. No. 6887), 0.9 mM Calcium chloride dihydrate (AppliChem, Cat. No. A3587), 50 mg/ml Streptomycin sulfate (AppliChem, Cat. No. A1852), 6 mg/ml Penicillin G potassium salt (AppliChem, Cat. No. A1837) and 1% Newborn Calf Serum (NBCS, Gibco™, Cat. No. 16010159). Cumulus-oocytes-complexes with multiple layers of compacted cumulus were matured in vitro in 1:1 DMEM High Glucose (Biowest, Cat. No. L0101-500) and Ham's F-12 Medium (Merck, Cat. No. F0815) supplemented with 60 μg/ml Penicilin G potassium salt (AppliChem, Cat. No. A1837), 50 ng/ml Streptomycin sulfate (AppliChem, Cat. No. A1852), 2.5 mM L-glutamine (AppliChem, Cat. No. A3704), 10% Fetal Bovine Serum (FCS, Gibco®, Lot 42Q0154K, Cat. No. 10270-106), 50 ng/ml murine Epidermal growth factor (EGF, SigmaAldrich, Cat. No. E4127), 10 I.E./ml Pregnant Mare's Serum Gonadotropin (PMSG, Intergonan® 240 I.E./ml, MSD Animal Health, Germany), 10 I.E./ml human Chorionic Gonadotropin (hCG, Ovogest® 300 I.E./ml, MSD Animal Health, Germany), 100 ng/ml human recombinant Insulin-like Growth Factor 1 (IGF1, R&D Systems, Cat. No. 291-G1), 5 ng/ml recombinant human FGF-basic (bFGF, Peprotech, Cat. No. 100-18B) for 40 h in humidified air with 5% CO₂ at 38.5° C.

### 6.8 Parthenogenetic Embryo Development Activation

After maturation, the oocytes were freed from cumulus cells by 5 min incubation with 0.1% Hyaluronidase (Sigma-Aldrich, Cat. No. H3506) in TL-Hepes 321+Ca²⁺medium composed of 114 mM Sodium chloride (NaCl, Sigma-Aldrich, Cat. No. S5886), 3.2 mM Potassium chloride (KCl, Sigma-Aldrich, P-5405), 2 mM Calcium chloride dihydrate (CaCl₂×2 H₂O; AppliChem, Cat. No. A3587), 0.4 mM Sodium dihydrogen monohydrate (NaH₂PO₄×H₂O, Merck, Cat. No. 106346), 0.5 mM Magnesium chloride hexahydrate (MgCl₂×6 H₂O, Roth, Cat. No. HN03.2), 2 mM Sodium hydrogen carbonate (NaHCO₃, Roth, Cat. No. HN01.2), 10 mM HEPES (Roth, Cat. No. 9105.3), 10 mM Sodium DL-lactate solution (60%) (SigmaAldrich, Cat. No. L1375), 100 U/L Penicilin G potassium salt (AppliChem, Cat. No. A1837), 50 mg/L Streptomycin sulfate (AppliChem, Cat. No. A1852), 0.25 mM Sodium Pyruvate (Sigma-Aldrich, Cat. No. P2256), 57 mM Sucrose (Merck, Cat. No. 107653) and 0.4% Bovine Serum Albumin (Sigma-Aldrich, Cat. No. A9647). After washing with TL-Hepes 321+Ca²⁺ medium oocytes with visible first polar body were exposed to a single pulse of 24 V for 45 μs in SOR activation medium (182.2 g/mol Sorbitol (Sigma-Aldrich, Cat. No. S1876), 158.2 g/mol Calcium acetate hydrate (Sigma-Aldrich, Cat. No. C4705), 214.5 g/mol Magnesium Acetate Tetrahydrate (Sigma-Aldrich, Cat. No. M5661), 0.1% Bovine Serum Albumin (Sigma-Aldrich, Cat. No. A9647)). Thereafter oocytes were incubated for 3 hours in 2 mM 6Dimethylaminopurine (6-DMAP, Sigma-Aldrich, Cat. No. D2629) in PZM-3 medium.

### 6.9 In Vitro Culture of Porcine Preimplantation Embryos

After activation, oocytes were cultured in PZM-3 medium at 39° C. in 5% CO₂ and 5% O₂ for 6 days. For isolation of ICM, porcine blastocysts from day 6 were cultured for an additional 24 h in D15 medium containing DMEM High Glucose (Biowest, Cat. No. L0101-500), and 2 mM L-Glutamine (AppliChem, Cat. No. A3704), 15% Fetal Bovine Serum (FCS, Gibco®, Lot 42Q0154K, Cat. No. 10270-106), 1% Penicillin/Streptomycin Solution (Corning, Cat. No. PS-B), 1% MEM Nonessential Amino Acids Solution (Corning, Cat. No. NEAA-B), 0.1 mM 2-mercaptoethanol (Sigma-Aldrich, Cat. No. M7522) supplemented with 1000 U/ml ESGRO® Recombinant Mouse LIF Protein (Millipore, Cat. No. ESG1107).

### 6.10 Isolation of ICMs from Porcine Parthenogenetic and In Vivo Collected Blastocysts

Porcine parthenogenetic blastocysts from day 7 and in vivo derived blastocysts from day 5 were used for the establishment of porcine PSC lines. Blastocysts were washed twice in TLHepes 296+Ca²⁺ medium composed of 114 mM Sodium Chloride (NaCl, Sigma-Aldrich, Cat. No. S5886), 3.2 mM Potassium chloride (KCl, Sigma-Aldrich, Cat. No. P-5405), 2 mM Calcium chloride dihydrate (CaCl₂×2 H₂O, AppliChem, Cat. No. A3587), 0.4 mM Sodium dihydrogen phosphate monohydrate (NaH₂PO₄×H₂O, Merck, Cat. No. 106346), 0.5 mM Magnesium chloride hexahydrate (MgCl₂×6 H₂O, Roth, Cat. No. HN03.2), 2 mM Sodium bicarbonate (NaHCO₃, Sigma-Aldrich, Cat. No. S4019), 10 mM HEPES (Roth, Cat. No. 9105.3), 10 mM Sodium DL-lactate solution (60%) (Sigma-Aldrich, Cat. No. L1375), 100 U/L Penicilin G potassium salt BioChemica (AppliChem, Cat. No. A1837), 50 mg/L Streptomycin sulfate BioChemica (AppliChem, Cat. No. A1852), 0.25 mM Sodium Pyruvate (Sigma-Aldrich, Cat. No. P2256), 32 mM Sucrose (Merck, Cat. No. 107653) and 0.4% Bovine Serum Albumin (BSA, Sigma-Aldrich, Cat. No. A9647). ICMs were separated from the trophectoderm in 100 μl drops of TL-Hepes 296+Ca² medium using ophthalmic scissors (Bausch & Lomb GmbH, Germany). Isolated ICMs were cultured on a monolayer of Mitomycin C-treated STO cells in pEPSCM medium, supplemented with 10 μM Y27632 (ROCKi, Tocris, Cat. No. 1254) for 7 days, until initial outgrowths could be observed. Subsequently, pEPSCM medium without ROCKi was used for further culture. Medium was changed every day. 12-14 days after plating, ICM colonies were mechanically removed from the STO feeder cells using fine-pulled glass capillary pipettes and reseeded onto fresh feeder cells. Growth of colonies was evaluated daily and approximately three days later cells began to form well-defined porcine EPSC^{Emb} colonies. These cells were sub-cultured using 0.05% trypsin-EDTA (GE Healthcare, Cat. No. L11-003) every 3-4 days.

### 6.11 In Vitro Chimera Assay

To investigate the developmental capacity of the derived cells lines, porcine EPSCs^{Emb} and EPSCs^{iPS} labelled with mCherry expression were injected into parthenogenetic blastocysts and the incidence of chimerism was assessed. Stem cells were detached from feeders with 0.05% trypsin-EDTA (GE Healthcare, Cat. No. L11-003) and re-suspended in Fetal Bovine Serum (FBS, Gibco®, Lot 42Q0154K, Cat. No. 10270-106). After centrifugation, stem cells were re-suspended and stored at room temperature in D15 medium supplemented with 1000 U/ml ESGRO® Recombinant Mouse LIF Protein (Millipore, Cat. No. ESG1107) and 10 μM Y27632 (ROCKi, Tocris, Cat. No. 1254). Small clumps containing 6-8 cells were injected into day 4 or day 6 old porcine parthenogenetic embryos with the aid of a piezo-driven micromanipulator (Zeiss, Eppendorf) in Opti-MEM® I (1×)+GlutamMAX™-I Reduced Serum Medium (Gibco®, Cat. No. 51985-026) supplemented with 10% FBS (Gibco®, Lot 42Q0154K, Cat. No. 10270-106). After injection, embryos were cultured in D15 medium supplemented with 1000 U/ml ESGRO® Recombinant Mouse LIF Protein (Millipore, Cat. No. ESG1107) and 10 μM Y27632 (ROCKi, Tocris, Cat. No. 1254) at 39° C. in 5% CO₂ and 5% O₂ for 24 hours (for blastocysts day 6) or 48 hours (for day 4 embryos). Non-injected porcine parthenogenetic embryos day 4 or day 6 cultured in the above medium were used as controls for embryo development.

### 6.12 In Vivo Chimera Assay

Procedures for superovulation, insemination and embryo collection were described above. Porcine morulae day 5 collected from eight gilts were stored in Opti-MEM® I (1×)+GlutamMAX™-I Reduced Serum Medium (Gibco®, Cat. No. 51985-026) supplemented with 10% FBS (Gibco®, Lot 42Q0154K, Cat. No. 10270-106) in thermostatically controlled incubator at 37° C. before injection. Porcine EPSC lines at passage 2-8 after mCherry⁺ colonies picking were used for the embryo injection. Porcine EPSCs were cultured either on mitotically inactivated STO feeder or MEFs cells in pEPSCM medium. Two days before injection the medium was switch to pEPSCM medium without WH-4-023 (SRCi, TOCRIS, Cat. No. 5413). One day before injection medium was replaced with pEPSCM medium without WH-4-023 and additionally supplemented with Heparin (5 ng/ml, R&D, Cat. No. 9041-08-1) and 10 ng/ml bFGF (SCI). Four hours before injection medium was replaced with pEPSCM medium without WH-4-023, supplemented with 5 ng/ml Heparin, 10 ng/ml bFGF (SCI—Stem Cell Institute, the University of Cambridge), 10 ng/ml Lif (SCI), 5 μM Y27632 (ROCKi, Tocris, Cat. No. 1254), 20 ng/ml Human Recombinant ACTIVIN A (StemCell Technologies, Cat. No. 78001) and 10% Fetal Bovine Serum (FCS, Gibco®, Lot 42Q0154K, Cat. No. 10270-106). For the injection EPSCs were detached from culture dish with 0.05% trypsin-EDTA (GE Healthcare, Cat. No. L11-003), carefully re-suspended and plated in 500 p1 drop of M15 medium supplemented with 50 μg/ml Vitamin C (Sigma, Cat. No. 49752), 0.1 μM CHIR99021 (GSK3i, TOCRIS, Cat. No. 4423), 20 ng/ml Human Recombinant Activin A (StemCell Technologies, Cat. No. 78001), 10 ng/ml bFGF (SCI), 10 ng/ml Lif (SCI), 5 ng/ml Heparin and 5 μM Y27632 (ROCKi, Tocris, Cat. No. 1254). Porcine embryos were washed once and placed in a 5000 drop of Opti-MEMO I (1×)+GlutamMAX™-I Reduced Serum Medium (Gibco®, Cat. No. 51985026) supplemented with 20 ng/ml Human Recombinant Activin A (StemCell Technologies, Cat. No. 78001), 10 ng/ml bFGF (SCI), 5 μM Y27632 (ROCKi, Tocris, Cat. No. 1254) and 10% FBS (Gibco®, Lot 42Q0154K, Cat. No. 10270-106). Injection drops were plated onto injection plate under phase-contrast inverted microscope (Axiovert 35M, Carl Zeiss, Oberkochen, Germany) equipped with a microinjection system (Transferman and CellTram Vario micromanipulators, Eppendorf) and covered with mineral oil. Stem cell clumps containing approximately 6-8 cells were injected between blastomeres of porcine morulae. Thereafter, embryos were washed twice in M15 medium supplemented with 50 μg/ml Vitamin C (Sigma-Aldrich, Cat. No. 49752), 0.1 μM CHIR99021 (GSK3i, TOCRIS, Cat. No. 4423), 20 ng/ml Human Recombinant Activin A (StemCell Technologies, Cat. No. 78001), 10 ng/ml bFGF (SCI), 10 ng/ml Lif (SCI), 5 ng/ml Heparin and 5 μM Y27632 (ROCKi, Tocris, Cat. No. 1254) and either incubated 4 hours until the embryo transfer or cultured overnight and then fixed for confocal microscopy analysis.

### 6.13 Evaluation of Chimerism in In Vitro Cultured Porcine Blastocysts

Porcine chimeric blastocysts were fixed in 3.7% formaldehyde solution (Honeywell Riedel-de Haen™, Cat. No. 1635) for 15 min at room temperature. Thereafter embryos were incubated with 0.2 μM SiR-DNA (Spirochrome, Switzerland) for 30 min at 37° C. to visualize the nuclei. Localization and proliferation of porcine stem cells in blastocysts were analysed using confocal screening microscope (LSM 510, Zeiss). Remaining embryos were stored in DPBS supplemented with 0.5% FBS (Gibco®, Lot 42Q0154K, Cat. No. 10270-106) and 1% Penicillin/Streptomycin Solution (Corning, Cat. No. PS-B) in 4° C. for future analysis.

### 6.14 Cryosectioning and Immunofluorescence Staining

Day 25-27 porcine fetuses were dissected from pregnant sows and cut into two halves along head-tail axis. The first half fetuses were fixed in 4% paraformaldehyde (Sigma, Cat. No. P6148) at 4° C. overnight and subsequently transferred to 30% sucrose solution (Sigma, Cat. No. 0389) for cryopreservation. The second halves were subjected to FACS and genotyping analysis. The fixed half fetuses were embedded in OCT compound (CellPath, Cat. No. 15212776) and frozen on dry ice. Sections (10 μm thick) were cut on a Leica cryostat. The sections were permeabilized with 0.1% Triton-100 (Sigma, Cat. No. T8787) for 30 minutes and then blocked for 30 minutes with 5% donkey serum (Sigma, Cat. No. D9663) and 1% BSA (Sigma, Cat. No. A2153). Co-immunofluorescences of mCherry and other antibodies were performed to check the co-localisation of injected donor porcine EPSCs expressing mCherry and host lineage markers. For immunofluorescence staining of cryosections of PGCLC EBs, the EBs were fixed in 4% PFA for about 4 hours or overnight at 4° C. and embedded in OCT compound for frozen sections. The thickness of each section was 10 μm. Sections were first permeabilized with 0.1% Triton and blocked with 5% donkey serum plus 1% BSA followed by incubations with primary antibodies for 1-2 hours at room temperature or overnight in a cold room. Fluorescence-conjugated secondary antibodies were used to incubate the slides at room temperature for 1 hour. After antibody treatment, samples were counter-stained with 10 μg/ml DAPI (Thermo Fisher Scientific, Cat. No. 62248) for 10 minutes to mark nuclei and were observed under a fluorescence microscope. The antibodies are listed in Supplementary Table 9.

### 6.15 Flow Cytometry of Dissected Porcine Chimera Tissues and EBs for PGCLCs

To analyse the contribution of donor mCherry⁺ porcine EPSCs in day 25-27 chimeras, the half fetuses were dissected into small pieces representing several body parts (head, trunk and tail). The dissected tissues and placenta were digested with 1.0 mg/ml collagenase IV (Thermo Fisher Scientific, Cat. No. 17104019) for 1-3 hours at 37° C. on a shaker. A pipette was used to blow the tissue blocks and dissociate them into single cells. The dissociated cells were filtered with a 35 μm nylon mesh (Corning, Cat. No. 352235) to remove tissues clumps. After centrifugation, the cells were fixed using Fixation Medium according to the manufacturers' manual (BD Cytofix, Cat. No. 554655) and the washed cells were stored at 4° C. in PBS supplemented with 0.1% NaN3 (Sigma, Cat. No. 199931) and 5% FBS (Gibco, Cat. No. 10270) before analysed with flow cytometry. All the samples were analysed using BD LSR Fortessa cytometer. 561 nm (610/20 bandpass filter) and 488 nm (525/50 bandpass filter) channels were used to detect mCherry and excluded autofluorescence. PGC EBs were trypsinized with 0.25% trypsin/EDTA Gibco, Cat. No. 25500-054) at 37° C. for 15 mins and stained with PerCP-Cy5.5 conjugated anti-TNAP antibody. 561 nm (610/20 bandpass filter) and 488 nm (710/50 bandpass filter) channels were used to detect NANOS3-H2BmCherry⁺/TNAP⁺ cells. FACS data were analysed by Flowjo software. The antibodies used in these experiments are listed in Supplementary Table 9.

### 6.16 Genotyping of Porcine Chimera Embryos

Genomic DNA of porcine fetuses were extracted from the dissociated cells of dissected body parts as described above and of placentas that were prepared for FACS using DNA Releasy kit (Anachem, Cat. No. LS02). Genomic DNA PCR of H2BmCherry was employed to detect the presences of donor cells. Amplification of a region in the porcine PRDM1 locus served as the genomic DNA quality and PCR control. All PCR primers are listed in Supplementary Table 10.

### 6.17 Differentiation of Porcine EPSCs to PGCLCs

For transcription factor mediated porcine PGCLC induction experiments, the piggyBac based PB-TRE-NANOG, PB-TRE BLIMP1, PB-TRE-TFAP2C and PB-CAG-SOX17-GR expression constructs were co-electroporated into the porcine NANOS3-2A-H2BmCherry reporter EPSCs^{emb} (Line K3, male) with PB-CAGG-rtTA-IRES-Puromycin and transposase expressing plasmids. pEPSCs^{Emb} harbouring the plasmids were selected by adding 0.3 μg/ml puromycin (Sigma, Cat. No. P8833) for two days. Thereafter the expressions of transgenic NANOG, BLIMP1 and TFAP2C were induced by 1.0 μg/ml Dox (Sigma, Cat. No.D9891) for indicated periods. As the SOX17 expressing plasmid has the hygromycin selection cassette, 150 μg/ml hygromycin (Gibco, Cat. No. 10687010) was used to select PB-CAG-SOX17-GR transfected cells. The SOX17 protein was fused with GR (human glucocorticoid receptor ligand-binding domain). This system allows inducing the nuclear translocation of SOX17 by addition of 2 μg/ml dexamethasone (Dex) (Sigma, Cat. No. D2915). For the pre-induction, pEPSCs^{Emb} were detached from the STO feeder layer by 0.1% Type 2 collagenase (Thermo Fisher Scientific, Cat. No. 17101015) without dissociation and seeded on gelatinised plates in M15 media supplemented with 5 μM ROCKi Y-27632 (Tocris, Cat. No. 1254), 20 μg/ml ACTIVIN A (SCI) and 1.0 μg/ml Dox or 1.0 mg/ml Dex. After the 12 hours of induction and pre-differentiation, the cells were collected using 0.25% trypsin/EDTA (Gibco, Cat. No. 25500-054) and plated to ultra-low attachment U-bottom 96-well plates (Corning, Cat. No. 7007) at a density of 5,000-6,000 cells/well in 100 ml PGCLC medium. 3-4 days later, the EBs were collected for analysis. PGCLC medium is composed of Advanced RPMI 1640 (GIBCO, Cat. No. 12633-12), 1% B27 Supplement (Thermo Fisher Scientific, Cat. No. 17504044), 1× glutamine penicillin-streptomycin (Thermo Fisher Scientific, Cat. No. 11140-050), 1×NEAA (Thermo Fisher Scientific, Cat. No. 10378-016), 0.1 mM 2-mercaptoethanol (Sigma, Cat. No. M6250) and the following cytokines: 500 ng/ml BMP2 (SCI), 10 ng/ml human LIF (SCI), 100 ng/ml SCF (R&D, Cat. No. 255-SC-010), 50 ng/ml EGF (R&D, Cat. No. 236-EG-200) and 10 μM ROCK inhibitor (Y-27632, Tocris, Cat. No. 1254).

For human PGCLCs, the PGC differentiation potential of two hEPSC lines are tested with the sequential induction method [6]. Human pre-mesoderm (pre-ME) was first induced in pre-ME media (Advanced RPMI 1640 Medium, 1% B27 supplement, 1×NEAA and 1× glutamine penicillin-streptomycin supplemented with 100 ng/ml Activin A (SCI), 3 μM CHIR99021 and 10 μM of ROCKi Y-27632) for 12 hours. Pre-ME were trypsinized into single cells and seeded into Corning Costar Ultra-Low attachment multi well 96-well plates (Corning, Cat. No. 7007) 4,000-5,000 cells per well in the 100 μM PGCLC medium which was used for porcine PGCLC induction. To improve the cell aggregation, in all PGCLC induction experiments, 0.25% (v/v) poly-vinyl alcohol (Sigma, Cat. No. 341584) are added in the basal medium.

### 6.18 Teratoma Assay of Porcine and Human EPSCs

Porcine and human EPSCs were re-suspended in PBS supplemented with 30% matrigel (Corning, Cat. No. 354230) and 5 μM Rock inhibitor Y-27632 (Tocris, Cat. No. 1254). 5×10⁶ porcine or human EPSCs were injected subcutaneously into both dorsal flanks of 8-weekold male NSG mice (NOD.Cg-Prkdcscid Il2rgtml Wjl/SzJ, The Jackson Laboratory) (100 ul per injection). Human and porcine EPSCs formed visible teratomas within 8 and 10 weeks. When the size of the teratomas reached 1.2 cm², they were dissected, fixed overnight in 10% phosphate-buffered formalin and embedded in paraffin before sectioning.

### 6.19 EB Formation Assay of Porcine and Human EPSCs

Porcine and human EPSCs were trypsinised and seeded in gelatinised 6-well plates at a density of 4×10⁶ cells/well for pre-differentiation. M15 media supplemented with 20 ng/ml ACTIVIN A (SCI) and 5 μM Rock inhibitor Y-27632 were used to culture the replated cells. The next day, the cells were detached using 0.25% trypsin/EDTA (Gibco, Cat. No. 25500-054) and plated to ultra-low cell attachment U-bottom 96-well plates (Corning, Cat. No. 7007) at a density of 5,000-6,000 cells/well in 200 μl M10 medium. After 7-8 days of growing, the EBs were collected for analysis. 0.25% (v/v) poly-vinyl alcohol (Sigma, Cat. No. 341584) was added in the medium to help cells aggregation.

### 6.20 Transfection of Porcine and Human EPSCs

pEPSCM without SRC inhibitor WH-4-023 (pEPSCM-SRCi) needs to be prepared in advance for pEPSCs transfection. Once pEPSCs reached 40-50% confluence, the media was switched to pEPSCM-SRCi and cells cultured for one more day (day −2). The next day (day −1), 5% FBS was added into pEPSCM-SRCi media and cells were cultured overnight. On the transfection day (day 0), porcine EPSCs were trypsinized with 0.25% trypsin/EDTA (Gibco, Cat. No. 25500-054) and dissociated into single cells with M10 media. After centrifugation, 1-1.5×10⁶ cells were resuspended in 100 μl Opti-MEM (Gibco, Cat. No. 31985062) containing 5-6 μg plasmid DNA. Amaxa Nucleofector machine (Lonza) was used to perform the electroporation with program A-023. After transfection, half of transfected cells were seeded on drug resistant STO feeders in 10-cm dishes and the pEPSCM supplemented with 5 μM ROCKi Y-27632 (Tocris, Cat. No. 1254) and 5% FBS were used to culture the transfected cells. Y-27632 and FBS was removed from the media on day 1. The drugs were added into pEPSCM media from day 2 to select the transfected colonies. The drug concentrations used for selection are: Puromycin (0.3 n/ml, Sigma, Cat. No. P8833); G418 (150 ng/ml, Gibco, Cat. No. 10131027); Hygromycin (150 ng/ml, Gibco, Cat. No. 10687010). After 3 days of selection (day 5), the medium was changed to pEPSCM-SRCi supplemented with drugs for continuous selection. The survived colonies were picked at day 7-8. During transfection and selection, the culture media should be refreshed daily. For human EPSCs transfection, 10% KSR and 5% FBS were added into hEPSCM to culture hEPSCs (70%-80% confluence) overnight before collection using 0.05% trypsin-EDTA the next day. M10 media was also used to dissociate the cells and neutralize the trypsin. Once centrifuged, 300-400 μl PBS solution containing plasmid DNA was used to resuspend the cells at a density of 10 million cells per ml. 300-400 μl cells/DNA mixture was taken out and added into 0.4-cm electroporation cuvettes for electroporation (Gene Pulser Xcell System; Bio-Rad; 320 V, 500 μF, 0.4-cm cuvettes). 5×10⁵ transfected cells were plated on drug resistant STO feeders in 10-cm dishes containing hEPSCM supplemented with 5 μM ROCKi Y-27632 (Tocris, Cat. No. 1254) and 10% KSR. Y-27632 and KSR were removed from the culture from day 1 and Puromycin was added for selection from day 2. Colonies were picked at around day 7-8. Follow the methods described above to expand the selected porcine and human EPSC colonies.

### 6.21 Crispr/Cas9 Mediated Genome-Editing in Porcine and Human EPSC Cells

To target an EF1a-H2BmCherry-iRES-Puro cassette to the porcine ROSA26 locus, the targeting vector with the cassette flanked by Rosa 5′ and 3′ homology arms was constructed. 5′ and 3′ homology arms were synthesised from IDT Company (650-bp 5′arm, Chr13: 65756272-65756923; 648-bp 3′arm, Chr13: 65755620-65756267). The sequence 5′CAATGCTAGTGCAGCCCTCATGG-3′ was designed as the target of gRNA/CAS9. After electroporation, Puromycin (0.3 μ/ml, Sigma, Cat. No. P8833) was used to select the targeted cells. Genotyping analysis of picked colonies revealed that the targeting efficiency was about 25%-30%. To investigate pPGCLC differentiation from pEPSCs, the T2A-H2BmCherry expression cassette was knocked-in immediately downstream and in frame with the coding sequence of porcine NANOS3. Homology arms were also synthesised from IDT company (699-bp 5′arm, chr2: 65275456-65276148; 699 bp-3′ arm chr2: 65274749-65275447). 20-bp (5′-TCCACTTCTGCCTAAGAGGCTGG-3′) sequence preceding the stop codon was targeted by gRNA/CAS9 to introduce the cut and mediate homologous recombination. After selection with G418 (150 μg/ml, Gibco, Cat. No. 10131027), genomic DNA was extracted from picked colonies and subjected to genotyping PCR revealing a comparable targeting efficiency of about 25%-30%. Karyotyping analysis of correctly targeted clones was performed to confirm normal karyotype in the clones used. The same strategy was employed to make human OCT4-T2A-H2B-Venus and CDX2-T2A-H2B-Venus reporter EPSC lines. For human OCT4 locus, homology arms are 619-bp 5′arm (chr6: 31164604-31165222) and 636-bp 3′arm (chr6:31163965-31164600). The gRNA/CAS9 targeting sequence is 5′ TCTCCCATGCATTCAAACTGAGG-3′. CDX2 homology arms are 478-bp 5′arm (chr13: 27963118-27963595) and 557-bp 3′arm (chr13: 27962558-27963114). The gRNA/CAS9 targeting sequence is 5′-CCGTCACCCAGTGACCCACCGGG-3′. For each electroporation, 5 μg plasmid DNA was used: 1.5 μg of CAS9, 1.5 μg of gRNA and 2 μg of donor vector.

### 6.22 Luciferase Assay

For the TOPflash assay, 2.0×10⁶ cells were transfected with 10 μg TOPflash plasmid. 5 μg pRL-TK (Renilla) vectors were also transfected for normalization. Cells were split 1:9 into a 24-well plate in pEPSCM and hEPSCM with or without XAV939 (WNTi, 2.5 μM, Cat. No. X3004) for 48 h. Cell lysates were collected for luciferase assays. For determining the regulation pattern of Oct4 expression in porcine EPSCs, 10 μg reporter constructs were electroporated into 1.5×10⁶ pEPSCs with 5 μg pRL-TK. Assays were performed 48 h later. All luciferase assays were performed using the Dual-Glo Luciferase Assay System (Promega, Cat. No. E2920).

### 6.23 Quantitative Real-Time PCR Analysis

Total RNA was isolated using an RNeasy Mini Kit (Qiagen, Cat. No. 74106) for cultured cells or RNeasy Micro Kit (Qiagen, Cat. No. 74034) for sorted NANOS3-mCherry⁺ cells. RNA was subsequently quantified and treated with gDNA WipeOut to remove genomic DNA. Complementary DNA (cDNA) was prepared using a QuantiTect Reverse Transcription Kit (Qiagen, Cat. No. 205311). RT-qPCR primers or TaqMan Gene Expression Assays (Life Technologies) are listed in Supplementary Table 10 and 11. ABsolute Blue qPCR ROX Mix (ABgene, Cat. No. AB4138B) were used for probe based qPCR assays and SYBR Green ROX qPCR Mastermix (Qiagen, Cat. No. 330523) were used for primer based qPCR assays. All qPCR reactions were performed on ABI 7900 HT Sequence Detection System (Life Technologies). Information on all primers and probes used for qPCR analysis are provided in Supplementary Table 10 and 11. Gene expression was determined relative to GAPDH using the A Ct method. Data are shown as the mean and s.d.

### 6.24 DMR Analysis

Bisulfite treatment was performed using the EpiTect Bisulfite Kit (Qiagen, Cat. No. 59124) according to the manufacturer's recommendations. Genomic DNA PCR for human ELF5 and porcine OCT4 and NANOG promoter regions was performed using primer pairs described before [7-9]. PCR products were cloned into pGEM-T Easy Vector (Promega, Cat. No. A1360) and sequenced from both ends. Randomly selected clones were sequenced with the M13 forward and M13 reverse primers for each promoter. The primers used in this analysis are provided in Supplementary Table 10.

### 6.25 Immunostaining for Cultured Cells

For dual staining of KRT7 with TFAP2C and GATA3, the differentiated hEPSCs were fixed in 4% paraformaldehyde (Sigma, Cat. No. P6148) solution, blocked with 3% goat serum and 1% BSA and incubated with mouse anti-KRT7 antibody at 4° C. overnight. Cells were then rinsed with PBS solution, incubated with Alexa 488-conjugated goat anti-mouse IgG secondary antibody (Abcam, Cat. No. AB150109) for 1 h at room temperature. After permeabilization with PBST (PBS solution with 0.3% Triton), cells were incubated with rabbit anti-TFAP2C and GATA3 antibodies at 4° C. overnight. The third day, cells were rinsed with PBST, incubated with Alexa 594-conjugated goat anti-rabbit IgG (Invitrogen, Cat. No. A21207) for 1 hour at room temperature, and counterstained with DAPI. For Tuj1, α-SMA, AFP and KRT7 immunostaining in differentiated porcine and human EPSCs, the cells were fixed and incubated with mouse-anti TUJ1, α-SMA, AFP and KRT7 antibodies, respectively, at 4° C. overnight. Cells were rinsed with PBS solution and incubated with Alexa 488-conjugated goat anti-mouse IgG (Abcam, Cat. No. AB150109) and 594-goat anti-mouse IgG (Invitrogen, Cat. No. A21207). After antibody treatment, samples were stained with 10 μg/ml DAPI (Thermo Fisher Scientific, Cat. No. 62248) to mark nuclei. For porcine and human pluripotency marker immunostaining, porcine and human EPSCs were fixed in 4% PFA/PBS solution, blocked in PBS solution with 3% goat serum (Sigma, Cat. No. G9023-10ML) and 1% BSA (Sigma, Cat. No. A2153) (for cell surface markers) or PBS solution with 3% goat serum, 1% BSA and 0.1% Triton (Sigam, Cat. No. T8787) (for intracellular markers, incubated with cell surface antibodies, SSEA-1, SSEA-4, Tra-1-60, Tra-1-81 or intracellular antibodies, OCT4, NANOG and SOX2 at 4° C. overnight. Cells were rinsed and incubated with Alexa 488 or 594-conjugated goat anti-mouse IgG, mouse IgM, rabbit IgG, and counterstained with DAPI. The antibodies used in these experiments is provided in Supplementary Table 9.

### 6.26 Western Blots

Whole-cell extracts were prepared from cells with indicated treatments in lysis buffer composed of 50 mM Tris-HCl (pH 7.5), 0.15M NaCl, 0.1% SDS, 1% Triton X-100, 1% sodium deoxycholate and complete mini EDTA free protease inhibitor cocktail (Roche Applied Science, Cat. No. 11836170001). The cells for the experiment were collected from the same batch of culture when the culture had reached 70-80% confluence. The biological replicates were included to allow the meaningful conclusions. 10 μg protein were used for electrophoresis and transferred to nitrocellulose membranes. Membranes were blocked with 5% milk and treated with antibodies.

Primary antibodies of mouse or rabbit anti AXIN1, SMAD2/3, p-SMAD2/3 and ALPHA-TUBULIN were used. Horseradish peroxidase-conjugated secondary antibodies against rabbit or mouse IgG were added. After antibody treatment, blots were developed using ECL Western Blotting Detection System (Thermo Fisher Scientific, Cat. No. 32106). The antibodies used in these experiments is provided in Supplementary Table 9.

### 6.27 Conversion of Human ESCs/iPSCs to EPSCs

For conversion of primed human ESC lines, 5×10⁴ trypsinized single cells were seeded on a 10-cm STO feeder plate in bFGF-containing standard media supplemented with 5 μM ROCK inhibitor Y-27632 (Tocris, Cat. No. 1254). Standard human ESC media: DMEM/F-12 (Gibco, Cat. No. 21331-020), 20% KnockOut Serum Replacement (KSR) (Gibco, Cat. No. 10828028), 1× Glutamine Penicillin-Streptomycin (Thermo Fisher Scientific, Cat. No. 11140-050), 1×NEAA (Thermo Fisher Scientific, Cat. No. 10378-016), and 0.1 mM 2-Mercaptoethanol (Sigma, Cat. No.M6250) and 10 ng/ml bFGF (SCI). One day later, medium was switched to hEPSCM and then refreshed every day. Following the initial differentiation of the majority cells, dome-shaped hEPSC colonies emerged in about 5-6 days, which could be expanded in bulk using 3-5 minutes treatment with 0.25% trypsin/EDTA (Gibco, Cat. No. 25500-054) on STO feeder layer at a density of 5×10⁴ cells/10-cm dish. 5-6 days later, stable dome-shaped single colonies could be picked and expanded following the method described above.

### 6.28 Reprogramming Human Fibroblasts to EPSCs

M20 media was used to culture human adult fibroblasts GM00013. The cells were collected by 0.25% trypsin/EDTA from ˜80% confluent T75 flask and washed once with PBS solution. The transfection was performed using an Amaxa Nucleofector machine (Lonza) according to the manufacturer's protocol (NHDF Nucleofector® Kit, Cat. No. VPD-1001). 5.0 μg of DNA were premixed in 100 μl transfection buffer. The DNA mixture consists of 2.0 μg of PB-TRE-hOCKS, 1.0 μg PB-TRE-RL, 1.0 μg PB-EF1a-transposase and 1.0 μg PB-EF1a-rtTA Among them, hOCKS were made with human cDNAs of OCT4, cMYC, KLF4 and SOX2 linked by 2A peptide. 1×10⁶ washed human adult fibroblasts were resuspended in 100 μl solution/DNA mixture and electroporated using program U-20. 0.2×10⁶ transfected cells were seeded on a STO feeder layer (10 cm-dish) in M15 media supplemented with 50 μg/ml Vitamin C (Sigma, Cat. No. 49752-100G). Dox (Sigma, Cat. No. D9891) was added in the media to 1.0 μg/ml final concentration to induce the reprogramming factors expression. After 12-14 days of induction, Dox was removed and the media was switched to hEPSCM for selecting the Dox independent human iPSC colonies. The survived colonies were picked to hEPSCM at ˜day 21 and expanded to stable iEPSC lines.

### 6.29 Differentiation of Human EPSCs to Trophoblast Lineages

hEPSCs were dissociated with 0.25% trypsin/EDTA and seeded in gelatinised 6-well plates at a density of 0.1×10⁶ cells/well. The cells were cultured in 20% KSR media supplemented with 5 μM ROCK inhibitor Y-27632 for 1 day. 20% KSR media: DMEM/F-12 (Gibco, Cat. No. 21331-020), 20% KnockOut Serum Replacement (KSR) (Gibco, Cat. No. 10828-028), 1× glutamine penicillin-streptomycin (Thermo Fisher Scientific, Cat. No. 11140-050), 1×NEAA (Thermo Fisher Scientific, Cat. No. 10378-016) and 0.1 mM 2-mercaptoethanol (Sigma, Cat. No. M6250). From the second day, different combinations of the TGFβ inhibitor SB431542 (10 μM, Tocris, Cat. No. 1514), BMP4 (50 ng/ml, R&D, Cat. No. 5020-BP) and the FGF receptor inhibitor PD173074 (0.1 μM, Tocris, Cat. No. 3044) were added into 20% KSR media to start the trophoblast differentiation. The cells were collected at indicated time points for analysis.

### 6.30 Derivation of Stable TSC Cell Lines from EPSCs

Single-dissociated hEPSCs and pEPSC^{Emb} were plated on 6-well plates pre-coated with 1 mg/ml Col W (Corning, Cat. No. 354233) at a density of 2,000 cells per well and cultured in hTSC media as described [10] with a minor modification. hTSC media: DMEM/F12 (Gibco, Cat. No. 21331-020) supplemented with 0.1m M2-mercaptoethanol, 0.2% FBS (Gibco, Cat. No. 10270), 0.5% Penicillin-Streptomycin, 0.3% BSA (Gibco, Cat. No. 15260037), 1% ITSX supplement (Gibco, Cat. No. 51500056), 50 μg/ml Vc (Sigma, Cat. No. 49752-100G), 50 ng/ml EGF (R&D, Cat. No. 236-EG-200), 2 μM CHIR99021 (GSK3i, TOCRIS, Cat. No. 4423), 0.5 μM A83-01 (TOCRIS, Cat. No. 2939), 1 μM SB431542 (Tocris, Cat. No. 1514), 0.8 μM VPA (STEMCELL, Cat. No. 72292) and 5 μM Y27632 (Tocris, Cat. No. 1254). After 7-9 days of culture, the colonies with TSC-like morphologies were picked, dissociated in TrypLE (Gibco, Cat. No. 12605036) and replated on the plate pre-coated with 1 mg/ml Col IV After 4-5 passage, the cells were collected for syncytiotrophoblast (ST) and extravillous trophoblast (EVT) differentiation tests with the methods described [10].

### 6.31 Porcine TSCs Embryos Injection

Two porcine TSCs lines (pK3-TSC-#1 and pK3-TSC-#3) transfected with H2BmCherry (EF1a-H2BmCherry and CAGG-H2BmCherry) were used for embryo injection experiments. Cells at passage 20 were briefly treated with TrypLE (Gibco, Cat. No. 12605036), gently tapped out from culture dish, and re-suspended in human TSCs medium. After centrifugation, TSCs were re-suspended in TL-Hepes 296 Ca-free medium composed of 114 mM Sodium Chloride (NaCl, Sigma-Aldrich, Cat. No. S5886), 3.2 mM Potassium chloride (KCl, Sigma-Aldrich, Cat. No. P-5405), 0.4 mM Sodium dihydrogen phosphate monohydrate (NaH2PO4×H2O, Merck, Cat. No. 106346), 0.5 mM Magnesium chloride hexahydrate (MgCl2×6 H2O, Roth, Cat. No. HN03.2), 2 mM Sodium bicarbonate (NaHCO₃, Sigma-Aldrich, Cat. No. S4019), 10 mM HEPES (Roth, Cat. No. 9105.3), 10 mM Sodium DL-lactate solution (60%) (Sigma-Aldrich, Cat. No. L1375), 100 U/L Penicilin G potassium salt BioChemica (AppliChem, Cat. No. A1837), 50 mg/L Streptomycin sulfate BioChemica (AppliChem, Cat. No. A1852), 0.25 mM Sodium Pyruvate (Sigma-Aldrich, Cat. No. P2256), 32 mM Sucrose (Merck, Cat. No. 107653), 0.4% Bovine Serum Albumin (BSA, Sigma-Aldrich, Cat. No. A9647) and 10 μM Y27632 (ROCKi, Tocris, Cat. No. 1254). For the injection, TSCs were incubated in 400 μl drops of TL-Hepes 296 Ca-free medium supplemented with 10 μM Y27632 (ROCKi, Tocris, Cat. No. 1254). Thereafter 8-10 single TSCs were injected into 6-day porcine parthenogenetic or IVF embryos with the aid of a piezo-driven micromanipulator (Zeiss, Eppendorf) in Opti-MEM I (1×)+GlutamMAX™-I Reduced Serum Medium (Gibco®, Cat. No. 51985-026) supplemented with 10% FBS (Gibco®, Lot42Q0154K, Cat. No. 10270-106)) and 10 μM Y27632 (ROCKi, Tocris, Cat. No. 1254). After injection, embryos were washed twice and cultured in D15 medium supplemented with 1000 U/ml ESGRO® recombinant mouse LIF protein (Millipore, Cat. No. ESG1107) and 10 μM Y27632 (ROCKi, Tocris, Cat. No. 1254) for 1-2 days at 39° C. in 5% CO2 and 5% 02. Thereafter embryos were fixed with 3.8% paraformaldehyde for 15 min at room temperature and stored in DPBS supplemented with 0.5% FBS (Gibco®, Lot 42Q0154K, Cat. No. 10270-106) and 1% Penicillin/Streptomycin Solution (Corning, Cat. No. PS-B) in 4° C.

### 6.32 Immunofluorescence Staining of Porcine Parthenogenetic Embryos Injected with TSCs

Fixed parthenogenetic blastocysts were washed three times in DPBS (Sigma, Cat. No. D5652-10X1L) supplemented with 0.5% FCS (Gibco®, Lot 42Q0154K, Cat. No. 10270-106) and permeabilized in DPBS supplemented with 0.5% Triton® X-100 (Merck, Cat. No. 108603) and 0.5% FCS for 1 h. Thereafter, embryos were washed three times in DPBS and blocked for 1 h at room temperature in blocking solution (co-staining GATA3/CDX2/mCherry: 5% horse serum (Sigma, Lot 14M175, Cat. No. H1270) and 0.2% Triton® X-100 in PBS. After blocking, embryos were incubated with primary antibodies diluted in DPBS and 0.5% FCS for overnight at 4° C. On the following day, embryos were transferred through several washes in DPBS supplemented with either 0.5% horse serum for GATA3/CDX2/mCherry. Secondary antibodies (mCherry: donkey anti-rabbit IgG (H+L) Alexa Fluor Plus 555, A32794, Invitrogen. GATA3/CDX2: donkey-anti-goat IgG (H+L) Alexa Fluor Plus 488, A32814, Invitrogen) were diluted in PBS supplemented with 0.5% horse serum at 1:1000 and the incubation occurred at room temperature for 1 h followed by washing as described above. To visualize nuclei, embryos were incubated in SiR-Hoechst (Spirochrome, SiR-DNA kit, Cat. No. SC007,) at 1:500 dilution in DPBS for 1 h at 37° C. and examined immediately using a confocal imaging system LSM510 (Carl Zeiss MicroImaging GmbH, Germany).

### 6.33 Porcine and Human TSC Lesion Assay

Porcine and human TS cells were dissociated with TrypLE (Gibco, Cat. No. 12605036) and re-suspended in PBS supplemented with 30% matrigel (Corning, Cat. No. 354230) and 10 μM Rock inhibitor Y-27632 (Tocris, Cat. No. 1254). 5×10⁶ porcine or human TSCs were injected subcutaneously into both dorsal flanks of 8-week-old male SCID mice (100 ul per injection). Human and porcine TSCs formed visible lesion within 7-10 days. The lesions were dissected, fixed overnight in 4% phosphate-buffered formalin and embedded in OCT compound (CellPath, Cat. No. 15212776) and paraffin for sectioning

### 6.34 ELISA

Enzyme-linked immunosorbent assay kits for human VEGF, P1GF, sFlt-1, CGA and sEng were obtained from R&D Systems and Human Chorionic Gonadotropin ELISA assay kits were sourced from ALPCO Diagnostics and performed according to the manufacturer's specifications.

### 6.35 RNA-Seq Analysis of Global Gene Expression in EPSCs and hTSCs

The cells for RNA preparation were collected from the same batch of culture when the culture had reached 70-80% confluence. The biological replicates were included to allow the meaningful conclusions. For human data, protein coding transcripts from GENCODE v27 were used, and transcripts from PAR Y regions were removed from the reference; for mouse data, protein coding transcripts from GENCODE vM16 were used; for porcine data, Ensembl build Sscrofa11.1 was used. Transcript fasta files were downloaded from GENCODE or Ensembl, and ERCC sequences were added into each build. Then the transcripts plus ERCC fasta files were indexed using salmon (version 0.9.1) [11], using the default parameter. When using GENCODE transcript reference, ‘---gencode’ flag was included during indexing to make sure salmon correctly handled the transcript id. For human naïve and primed ESC RNA-seq [12], fastq files were downloaded from ENA (Study accession PRJNA326944); for human embryo single cell data fastq files were downloaded from ENA (Study accession PRJEB8994) [13, 14]. For mouse EPSC data, fastq files from the previous study [15] were used. All the reads were directly quantified against the transcriptomes of the corresponding species using salmon (version 0.9.1) with the flags ‘--useVBOpt --numBootstraps 100 --posBias --seqBias --gcBias -1 ISR -g gene_map.tsv’ where gene_map.tsv was a tab delimited file mapping transcript ids to gene ids to get gene level expression values. The expression levels of each selected histone gene in different types of human cells and early embryos were extracted from expression matrix and visualized as a heatmap generated by GraphPad Prism 7.04 (https://www.graphpad.com/scientific-software/prism/). Gene expression values are linearly transformed into colours (as indicated by the colour legend below each matrix) in which blue colour represents low gene expression, red represents higher gene expression and no colour is equivalent to the highest level of the gene that was expressed. For single cell RNA-seq, an extra quality control step is added, where cells with less than 10,000 total reads, or less than 4,000 detected genes (at least 1 read), or more than 80% of reads mapped to ERCC or more than 60% of non-mappable reads were removed before downstream analyses.

### 6.36 Batch Correction, Principal Component Analysis (PCA) and Cross-Species Comparison

Gene count from each sample was collected together, and log 10 transformed. Then batch effect (batches here mean different studies) and sequencing depth (total number of reads per sample) were regressed out using the “regress out” function from the NaiveDE package (https://github.com/Teichlab/NaiveDE/tree/master/NaiveDE). Principal component analyses were done on the regressed matrix using scikit-learn (Scikit-learn: Machine Learning in Python, Pedregosa et al., JMLR 12, pp. 2825-2830, 2011.). For cross-species comparison, only the one-to-one orthologous genes were used.

### 6.37 RNA-Seq Analysis of Human EPSC Differentiation to Trophoblasts

Gene expression matrix: reference index was created based on hg38 from GENCODE database [16]. Gene expression matrices for H1-ESC, H1-EPSC, hiPSC-EPSC, PHTu and PHTd were generated using Salmon [11] with following parameters: salmon quant --noversion-check -q -p 6 --useVBOpt --numBootstraps 100 --posBias --seqBias --gcBias. t-SNE (t-distributed stochastic neighbor embedding) analysis: R package ‘Rtsne’ was used for the dimension reduction of gene expression matrices (genes with maximum TPM<=1 were filtered out) and the corresponding result was visualized using a custom R script. Pearson correlation: the RNA-seq data for reference tissues was downloaded from Chang et al. paper [17], the data for reference cells (uESCs, uPHTs, dESCs, dPHTs) was downloaded from Yabe et al. paper [18]. A list of tissue specific genes (n=2293) defined by Chang et al. were selected for Pearson correlation coefficients analysis. Pairwise calculation was performed between the provided data (H1-ESC, H1-EPSC and hiPSC-EPSC) and external references. The result was visualized as a heatmap with high similarity in red colors while low similarity in blue colors. Expression dynamics of 37 trophoblast marker genes were analysed. The expression levels of each marker gene were extracted from expression matrix and normalized using the following method. The TPM of a given gene was divided by the highest gene expression level of that gene in a row (12 data points for each cell line, in total 36 values for H1-ESC, H1-EPSC and hiPSC-EPSC). Through this method, each TPM was transformed into a value between 0 and 1. The overall gene signatures were plotted as a heatmap using color keys ranging from blue (lowly expressed genes) to red (highly expressed genes). The cells for RNA preparation were collected from the same batch of culture when the culture had reached 70-80% confluence. The biological replicates were included to allow the meaningful conclusions.

### 6.38 PCA Analysis of Human TSC RNAseq

“Factoextra” R package is applied for PCA analysis and “limma” R package for batch effect removal. Genes whose TPM values were lower than 1 in all samples were removed from the TPM expression matrix.

### 6.39 Construction of Single-Cell RNAseq Libraries

The single-cell mRNA-seq library was generated following the SMART-seq2 protocol described [19]. In short, single porcine and human EPSCs were sorted into 96-well plates prefilled with lysis buffer and external RNA spike-ins (Ambion) (1:500,000). First-strand synthesis and template-switching were then performed, followed by 25-cycle of pre-amplification. Complementary DNAs were purified by AMPure XP magnetic beads (Agencourt) using an automated robotic workstation (Zephyr). Quality of cDNAs was checked with the Bioanalyzer (Agilent) using high sensitivity DNA chip. Multiplex (96-plex) libraries were constructed and amplified using Nextera XT library preparation kit (Illumina). The libraries were then pooled and purified with AMPure XP magnetic beads. The quality of the library was then assessed by the Bioanalyzer (Agilent) before submission to the DNA sequencing pipeline at the Wellcome Trust Sanger Institute. Pair-ended 75-bp reads were generated by HiSeq2000 sequencers. Porcine and human scRNA seq data can be downloaded from: ftp://ngs.sanger.ac.uk/production/teichmann/xi/xuefei_epsc/single_cell_expr_matrix

Expression violin plot for all genes from scRNAseq: Porcine EPSCs: ftp://ngs.sanger.ac.uk/production/teichmann/xi/xuefei_epsc/porcine_sc_vplot/index.htm
Human EPSCs: ftp://ngs.sanger.ac.uk/production/teichmann/xi/xuefei_epsc/human_sc_vplot/index.html

### 6.40 ChIP-Seq Analysis of Histone Modification Profiles in EPSCs

The H3K4me3, H3K27me3, H3K27ac and input ChIP libraries of porcine and human EPSCs were prepared based on a modified ChIP protocol from Lee et al [20]. In short, about 20 million cells were cross-linked in 1% formaldehyde for 10 mins at room temperature. Cross-linking was then quenched with 0.125 M glycine for 5 minutes at room temperature. Cell pellets were washed with PBS, snap frozen by liquid nitrogen and stored in −80° C. until further processing. Chromatin was sheared by Bioruptor Pico (Diagenode) for 5-7 cycles: 30 sec on and off cycles. Immunoprecipitation were performed with 1 μg antibody pre-washed and pre-attached to protein A Dynaebeads (Invitrogen, Cat. No. 10002D) overnight at 4° C. Antibodies: H3K4me3, H3K27me3, H3K27ac are listed in Supplemental Table 9. The beads were then washed and cross-linking was reversed with the elution buffer at 65° C. for 4 hours. Immunoprecipitated DNAs were purified with proteinase K digestion and the Qiagen minElute PCR Purification kit (Qiagen, Cat. No. 28004). The multiplex sequencing libraries were prepared with the microplex library construction kit (Diagenode, Cat. No. 005010014) following manufacturer's instruction. DNA was amplified for 11 cycles and the quality of the library was checked on a bioanalyzer (Ailgent) using a high sensitivity DNA kit. Library concentration was check by qPCR using KAPA Library Quantification Kit (KK4824), and equal molar of different libraries were pooled and sequenced on 2 lanes of HiSeq2500. 50 base pair single end reads were mapped to the UCSC reference genomes (build susScr11 for porcine and hg38 for human) using bowtie2 (version 2.3.4) [21] with default setting. For the human reference hg38, all the alternative loci were removed (chr*_alt) before mapping. Reads mapped to the mitochondrial genome were removed, and reads mapped to the nuclear genome were filtered by samtools [22] with flags ‘-q 30’ to filter reads with relatively low mapping quality (MAPQ less than 30). For the ChIP-seq data from human naïve and primed ESCs [12], raw reads were downloaded from ENA (Study accession PRINA255308) and processed in the same manner. Peak calling was performed using MACS2 (2.1.1.20160309) [23]. For identification of enriched regions of punctate marks (H3K4me3 and H3K27ac) from porcine samples, peak calling was performed with flags ‘-t chip.bam -c input.bam -g 2.7e9 -q 0.01 -f BAM --nomodel -extsize 200 -B --SPMR’. For identification of enriched regions of broad marks (H3K27me3), peak calling was performed with flags ‘-t chip.bam -c input.bam -g 2.7e9 -q 0.01 -f BAM -nomodel --extsize 200 -B --SPMR --broad’. For human data, peak calling was done in the same way, with a change of genome size ‘-g hs’ during the peak calling. The resulting bedGraph files were converted to bigWig files using the script bdg2bw (https://gist.github.com/j132587/34370c995460f9d5ad65). The bigWig files were visualised using UCSC genome browser[24]. To compare the H3K4me3 signal around naive and primed genes, the differentially expressed gene list between human naive and primed ESCs was downloaded from the Supplementary Table of Theunissen et al. [12]. Genes were sorted by log 2 fold change, and then the top 1000 naïve or primed genes were selected. The H3K4me3 signals of human EPSCs were directly quantified around the transcriptional start sites of those 2000 genes using HOMER (v4.9) [25]. For porcine data, the one-to-one orthologues of those 2000 genes were first extracted from ensembl genome browser [26], and then porcine H3K4me3 signals were quantified in the same way as in human. The cells for histone modification profiles were collected from the same batch of culture when the culture had reached 70-80% confluence. The biological replicates were included to allow the meaningful conclusions.

### 6.41 Whole Genome DNA Methylation Analysis

DNA methylation levels were measured by whole genome bisulfate sequencing [27]. DNA was purified (Qiagen Blood DNA Extraction kit), sonicated using a covaris sonicator. Approximately 500 ng DNA per sample was processed using the NEBNext Ultra DNA library prep kit (NEB E7370) using methylated adapters (NEB or Illumina). Bisulfite conversion was performed using EZ DNA methylation Gold kit (Zymo) prior to final PCR amplification. Libraries were sequenced using Illumina MiSeq platform to generate 100 bp paired end reads. Raw sequence reads were trimmed to remove both poor quality calls and adapters using Trim Galore (v0.4.1, www.bioinformatics.babraham.ac.uk/projects/trim_galore/, Cutadapt version 1.8.1, parameters: --paired) and aligned to the human or porcine genome using Bismark v0.18.2 (Krueger and Andrews, 2011). Data were quantitated using SeqMonk (www.bioinformatics.babraham.ac.uk/projects/seqmonk/) using 500 CpG running windows and a minimum coverage of 100 CpG per window. The cells in this analysis were collected from the same batch of culture when the culture had reached 70-80% confluence.

### 6.42 Statistical Analysis

No statistical methods were used to predetermine sample size. The experiments were not randomized. The investigators were not blinded to allocation during experiments and outcome assessment. The statistical analysis was conducted with Microsoft Excel or Prism 7.04 (GraphPad). P values were calculated using a two-tailed Student's t-test.

### 6.43 Data Availability

Sequencing data are deposited into ArrayExpress, and the accession numbers are E-MTAB-7252 (CMP-seq), E-MTAB-7253 (bulk RNA-seq) and E-MTAB-7254 (single cell RNA-seq). Human cell sequencing raw data (including ChIP-seq and bulk/single cell RNA-seq) files can be accessed via ftp://ngs.sangerac.uk/production/teichmann/xi/xuefei_epsc/human_fastqi; Porcine cell sequencing raw data (including ChIP-seq and bulk/single cell RNA-seq) files can be accessed via ftp://ngs.sangerac.uk/production/teichmann/xi/xuefei_epsc/pig_fastq/. All other relevant data are available from the corresponding author on request.

### REFERENCES

1 Yang, J. et al. Establishment of mouse expanded potential stem cells. Nature 550, 393-397, doi:10.1038/nature24052 (2017).
2 Ezashi, T., Yuan, Y. & Roberts, R M. Pluripotent Stem Cells from Domesticated Mammals. Annual review of animal biosciences 4, 223-253, doi:10.1146/annurev-animal-021815-111202 (2016).
3 Evans, M. J., Notarianni, E., Laurie, S. & Moor, R. M. Derivation and preliminary characterization of pluripotent cell lines from porcine and bovine blastocysts. Theriogenology 33: 125-128., 33 (1990).
4 Piedrahita, J. A., Anderson, G. B. & Bondurant, R. H. Influence of feeder layer type on the efficiency of isolation of porcine embryo-derived cell lines. Theriogenology 34, 865-877 (1990).
5 Ropeter-Scharfenstein, M., Neubert, N., Prelle, K. & Holtz, W. Identification, isolation and culture of pluripotent cells from the porcine inner cell mass. Joournal of Animal Breeding and Genetics 113, 427-436 (1996).
6 Notarianni, E., Laurie S, N. A., Sathasivam K., NG, A. & Sathasivam, K. Incorporation of cultured embryonic cells into transgenic and chimeric, porcine fetuses. Int J Dev Biol. 41, 537-540 (1997).
7 Chen, L. R. et al. Establishment of pluripotent cell lines from porcine preimplantation embryos. Theriogenology 52, 195-212, doi:10.1016/50093-691X(99)00122-3 (1999).
8 Shiue, Y L. et al. In vitro culture period but not the passage number influences the capacity of chimera production of inner cell mass and its deriving cells from porcine embryos. Animal reproduction science 93, 134-143 (2006).
9 Brevini, T. A. et al. Culture conditions and signalling networks promoting the establishment of cell lines from parthenogenetic and biparental porcine embryos. Stem cell reviews 6, 484-495, doi:10.1007/s12015-010-9153-2 (2010).
10 Vassiliev, I. et al. In vitro and in vivo characterization of putative porcine embryonic stem cells. Cellular reprogramming 12, 223-230, doi:10.1089/cell.2009.0053 (2010).
11 Haraguchi, S., Kikuchi, K., Nakai, M. & Tokunaga, T. Establishment of self-renewing porcine embryonic stem cell-like cells by signal inhibition. The Journal of reproduction and development 58, 707-716 (2012).
12 Park, J. K. et al. Primed pluripotent cell lines derived from various embryonic origins and somatic cells in pig. PLoS One 8, e52481, doi:10.1371/journal.pone. 0052481 (2013).
Hou, D. R. et al. Derivation of Porcine Embryonic Stem-Like Cells from In Vitro-Produced Blastocyst-Stage Embryos. Sci Rep 6, 25838, doi:10.1038/srep25838 (2016).
14 Xue, B. et al. Porcine Pluripotent Stem Cells Derived from IVF Embryos Contribute to Chimeric Development In Vivo. PLoS One 11, e0151737, doi:10.1371/journal.pone.0151737 (2016).
15 Ma, Y, Yu, T., Cai, Y. & Wang, H. Preserving self-renewal of porcine pluripotent stem cells in serum-free 3i culture condition and independent of LIF and b-FGF cytokines. Cell death discovery 4, 21, doi:10.1038/s41420-017-0015-4 (2018).
16 Esteban, M. A. et al. Generation of induced pluripotent stem cell lines from Tibetan miniature pig. J Biol Chem 284, 17634-17640, doi:10.1074/jbc.M109.008938 (2009).
17 Ezashi, T. et al. Derivation of induced pluripotent stem cells from porcine somatic cells. Proc Natl Acad Sci USA 106, 10993-10998, doi:10.1073/pnas. 0905284106 (2009).
18 Roberts, R. M., Telugu, B. P & Ezashi, T. Induced pluripotent stem cells from swine (Sus scrofa): why they may prove to be important. Cell cycle 8, 3078-3081, doi:10.4161/cc.8.19.9589 (2009).
19 Wu, Z. et al. Generation of porcine induced pluripotent stem cells with a drug-inducible system. Journal of molecular cell biology 1, 46-54, doi:10.1093/jmcb/mjp003 (2009).
20 Telugu, B. P, Ezashi, T. & Roberts, R. M. Porcine induced pluripotent stem cells analogous to naive and primed embryonic stem cells of the mouse. The International journal of developmental biology 54, 1703-1711, doi:10.1387/ijdb.103200bt (2010).
21 West, F. D. et al. Porcine induced pluripotent stem cells produce chimeric offspring. Stem cells and development 19, 1211-1220, doi:10.1089/scd.2009.0458 (2010).
22 Zhang, W. et al. Pluripotent and Metabolic Features of Two Types of Porcine iPSCs Derived from Defined Mouse and Human ES Cell Culture Conditions. PLoS One 10, e0124562, doi:10.1371/journal.pone.0124562 (2015).
23 Petkov, S., Glage, S., Nowak-Imialek, M. & Niemann, H. Long-Term Culture of Porcine Induced Pluripotent Stem-Like Cells Under Feeder-Free Conditions in the Presence of Histone Deacetylase Inhibitors. Stem cells and development 25, 386-394, doi:10.1089/scd.2015.0317 (2016).
24 Wang, H. et al. Induction of Germ Cell-like Cells from Porcine Induced Pluripotent Stem Cells. Sci Rep 6, 27256, doi:10.1038/srep27256 (2016).
25 Lai, S. et al. Generation of Knock-In Pigs Carrying Oct4-tdTomato Reporter through CRISPR/Cas9-Mediated Genome Engineering. PLoS One 11, e0146562, doi:10.1371/journal.pone.0146562 (2016).
26 Ying, Q. L. et al. The ground state of embryonic stem cell self-renewal. Nature 453, 519-523, doi:10.1038/nature06968 (2008).
27 Du, X. et al. Barriers for Deriving Transgene-Free Porcine iPS Cells with Episomal Vectors. Stem Cells 33, 3228-3238, doi:10.1002/stem.2089 (2015).
28 Chen, H. et al. Erk signaling is indispensable for genomic stability and self-renewal of mouse embryonic stem cells. Proc Natl Acad Sci USA 112, E5936-E5943 (2015).
29 Theunissen, T. W. et al. Systematic identification of culture conditions for induction and maintenance of naive human pluripotency. Cell Stem Cell 15, 471-487, doi:10.1016/j.stem.2014.07.002 (2014).
30 Takashima, Y. et al. Resetting Transcription Factor Control Circuitry toward Ground-State Pluripotency in Human. Cell 158, 1254-1269, doi:10.1016/j.cell.2014.08.029 (2014).
31 Hayashi, K., Ohta, H., Kurimoto, K., Aramaki, S. & Saitou, M. Reconstitution of the mouse germ cell specification pathway in culture by pluripotent stem cells. Cell 146, 519-532, doi:10.1016/j.cell.2011.06.052 (2011).
32 Irie, N. et al. SOX17 is a critical specifier of human primordial germ cell fate. Cell 160, 253-268, doi:10.1016/j.cell.2014.12.013 (2015).
33 Kobayashi, T. et al. Principles of early human development and germ cell program from conserved model systems. Nature 546, 416-420, doi:10.1038/nature22812 (2017).
34 Julaton, V. T. & Reijo Pera, R. A. NANOS3 function in human germ cell development. Hum Mol Genet 20, 2238-2250, doi:10.1093/hmg/ddr114 (2011).
35 Gkountela, S. et al. The ontogeny of cKIT+ human primordial germ cells proves to be a resource for human germ line reprogramming, imprint erasure and in vitro differentiation. Nat Cell Biol 15, 113-122, doi:10.1038/ncb2638 (2013).
36 Thomson, J. A. et al. Embryonic stem cell lines derived from human blastocysts. Science 282, 1145-1147 (1998).
37 Warmflash, A., Sorre, B., Etoc, F., Siggia, E. D. & Brivanlou, A. H. A method to recapitulate early embryonic spatial patterning in human embryonic stem cells. Nat Methods 11, 847-854, doi:10.1038/nmeth.3016 (2014).
38 Camarasa, M. V et al. Derivation of Man-1 and Man-2 research grade human embryonic stem cell lines. In vitro cellular & developmental biology. Animal 46, 386-394, doi:10.1007/s11626-010-9291-5 (2010).
39 Ye, J. et al. High quality clinical grade human embryonic stem cell lines derived from fresh discarded embryos. Stem cell research & therapy 8, 128, doi:10.1186/s13287-017-0561-y (2017).
40 International Stem Cell, I. et al. Characterization of human embryonic stem cell lines by the International Stem Cell Initiative. Nature biotechnology 25, 803-816, doi:10.1038/nbt1318 (2007).
41 Koyanagi-Aoi, M. et al. Differentiation-defective phenotypes revealed by large-scale analyses of human pluripotent stem cells. Proc Natl Acad Sci USA 110, 20569-20574, doi:10.1073/pnas.1319061110 (2013).
42 Theunissen, T. W. et al. Molecular Criteria for Defining the Naive Human Pluripotent State. Cell Stem Cell 19, 502-515, doi:10.1016/j.stem.2016.06.011 (2016).
43 Yang, Y et al. Derivation of Pluripotent Stem Cells with In Vivo Embryonic and Extraembryonic Potency. Cell 169, 243-257 e225, doi:10.1016/j.cell.2017.02.005 (2017).
44 Yan, L. et al. Single-cell RNA-Seq profiling of human preimplantation embryos and embryonic stem cells. Nature structural & molecular biology 20, 1131-1139, doi:10.1038/nsmb.2660 (2013).
45 Dang, Y et al. Tracing the expression of circular RNAs in human pre-implantation embryos. Genome Biol 17, 130, doi:10.1186/s13059-016-0991-3 (2016).
46 Blakeley, P. et al. Defining the three cell lineages of the human blastocyst by single-cell RNA-seq. Development 142, 3613, doi:10.1242/dev.131235 (2015).
47 Chen, Y, Blair, K. & Smith, A. Robust Self-Renewal of Rat Embryonic Stem Cells Requires Fine-Tuning of Glycogen Synthase Kinase-3 Inhibition. Stem Cell Reports 1, 209-217, doi:10.1016/j.stemcr.2013.07.003 (2013).
48 Xu, R. H. et al. BMP4 initiates human embryonic stem cell differentiation to trophoblast. Nature biotechnology 20, 1261-1264, doi:10.1038/nbt761 (2002).
49 Amita, M. et al. Complete and unidirectional conversion of human embryonic stem cells to trophoblast by BMP4. Proc Natl Acad Sci USA 110, E1212-1221, doi:10.1073/pnas.1303094110 (2013).
50 Yabe, S. et al. Comparison of syncytiotrophoblast generated from human embryonic stem cells and from term placentas. Proc Natl Acad Sci USA 113, E2598-2607, doi:10.1073/pnas.1601630113 (2016).
Chilosi, M. et al. Differential expression of p57kip2, a maternally imprinted cdk inhibitor, in normal human placenta and gestational trophoblastic disease. Laboratory investigation; a journal of technical methods and pathology 78, 269-276 (1998).
52 Zhang, P., Wong, C., DePinho, R. A., Harper, J. W. & Elledge, S. J. Cooperation between the Cdk inhibitors p27(KIP1) and p57(KIP2) in the control of tissue growth and development. Genes Dev 12, 3162-3167 (1998).
53 Okae, H. et al. Derivation of Human Trophoblast Stem Cells. Cell Stem Cell 22, 50-63 e56, doi:10.1016/j.stem.2017.11.004 (2018).
54 Lee, C. Q. et al. What Is Trophoblast? A Combination of Criteria Define Human First-Trimester Trophoblast. Stem Cell Reports 6, 257-272, doi:10.1016/j.stemcr.2016.01.006 (2016).
55 Hemberger, M., Udayashankar, R, Tesar, P, Moore, H. & Burton, G. J. ELF5-enforced transcriptional networks define an epigenetically regulated trophoblast stem cell compartment in the human placenta. Hum Mol Genet 19, 2456-2467, doi:10.1093/hmg/ddq128 (2010).
56 Ng, R. K. et al. Epigenetic restriction of embryonic cell lineage fate by methylation of Elf5. Nat Cell Biol 10, 1280-1290, doi:10.1038/ncb1786 (2008).
57 Huang, S. M. et al. Tankyrase inhibition stabilizes axin and antagonizes Wnt signalling. Nature 461, 614-620, doi:10.1038/nature08356 (2009).
58 Thorsell, A. G. et al. Structural Basis for Potency and Promiscuity in Poly(ADP-ribose) Polymerase (PARP) and Tankyrase Inhibitors. Journal of medicinal chemistry 60, 1262-1271, doi:10.1021/acs.jmedchem.6b00990 (2017).
59 Hassa, P. O. & Hottiger, M. O. The diverse biological roles of mammalian PARPS, a small but powerful family of poly-ADP-ribose polymerases. Front Biosci 13, 3046-3082 (2008).
60 Hemberger, M. et al. Parp1-deficiency induces differentiation of ES cells into trophoblast derivatives. Dev Biol 257, 371-381 (2003).
61 Koh, D. W. et al. Failure to degrade poly(ADP-ribose) causes increased sensitivity to cytotoxicity and early embryonic lethality. Proc Natl Acad Sci USA 101, 17699-17704, doi:10.1073/pnas.0406182101 (2004).
62. Nowak-Imialek, M., et al., Oct4-enhanced green fluorescent protein transgenic pigs: a new large animal model for reprogramming studies. Stem Cells Dev, 2011. 20(9): p. 1563-75.
63. Lai, S., et al., Generation of Knock-In Pigs Carrying Oct4-tdTomato Reporter through CRISPR/Cas9-Mediated Genome Engineering. PLoS One, 2016. 11(1): p. e0146562.
64. Petkov, S., et al., Long-Term Culture of Porcine Induced Pluripotent Stem-Like Cells Under Feeder-Free Conditions in the Presence of Histone Deacetylase Inhibitors. Stem Cells Dev, 2016. 25(5): p. 386-94.
65. Wang, W., et al., Rapid and efficient reprogramming of somatic cells to induced pluripotent stem cells by retinoic acid receptor gamma and liver receptor homolog 1. Proc Natl Acad Sci USA, 2011. 108(45): p. 18283-8.
66. Petersen, B., et al., Development and validation of a highly efficient protocol of porcine somatic cloning using preovulatory embryo transfer in peripubertal gilts. Cloning Stem Cells, 2008. 10(3): p. 355-62.
67. Kobayashi, T., et al., Principles of early human development and germ cell program from conserved model systems. Nature, 2017. 546(7658): p. 416-420.
68. Nowak-Imialek, M., et al., Preferential loss of porcine chromosomes in reprogrammed interspecies cell hybrids. Cell Reprogram, 2010. 12(1): p. 55-65.
69. Lee, C. Q., et al., What Is Trophoblast? A Combination of Criteria Define Human FirstTrimester Trophoblast. Stem Cell Reports, 2016. 6(2): p. 257-72.
70. Miyamoto, K., et al., Cell-free extracts from mammalian oocytes partially induce nuclear reprogramming in somatic cells. Biol Reprod, 2009. 80(5): p. 935-43.
71. Okae, H., et al., Derivation of Human Trophoblast Stem Cells. Cell Stem Cell, 2018. 22(1): p. 50-63 e6.
72. Patro, R., et al., Salmon provides fast and bias-aware quantification of transcript expression. Nat Methods, 2017. 14(4): p. 417-419.
73. Theunissen, T. W., et al., Systematic identification of culture conditions for induction and maintenance of naive human pluripotency. Cell Stem Cell, 2014. 15(4): p. 471-87.
74. Dang, Y, et al., Tracing the expression of circular RNAs in human pre-implantation embryos. Genome Biol, 2016. 17(1): p. 130.
75. Yan, L., et al., Single-cell RNA-Seq profiling of human preimplantation embryos and embryonic stem cells. Nat Struct Mol Biol, 2013. 20(9): p. 1131-9.
76. Yang, J., et al., Establishment of mouse expanded potential stem cells. Nature, 2017. 550(7676): p. 393-397.
77. Harrow, J., et al., GENCODE: the reference human genome annotation for The ENCODE Project. Genome Res, 2012. 22(9): p. 1760-74.
78. Chang, C. W., et al., Identification of human housekeeping genes and tissue-selective genes by microarray meta-analysis. PLoS One, 2011. 6(7): p. e22859.
79. Yabe, S., et al., Comparison of syncytiotrophoblast generated from human embryonic stem cells and from term placentas. Proc Natl Acad Sci USA, 2016. 113(19): p. E2598-607.
80. Picelli, S., et al., Full-length RNA-seq from single cells using Smart-seq2. Nat Protoc, 2014. 9(1): p. 171-81.
81. Lee, T. I., S. E. Johnstone, and R. A. Young, Chromatin immunoprecipitation and microarray based analysis of protein location. Nat Protoc, 2006. 1(2): p. 729-48.
82. Langmead, B. and S. L. Salzberg, Fast gapped-read alignment with Bowtie 2. Nat Methods, 2012. 9(4): p. 357-9.
83. Li, H., et al., The Sequence Alignment/Map format and SAMtools. Bioinformatics, 2009. 25(16): p. 2078-9.
84. Zhang, Y, et al., Model-based analysis of ChIP-Seq (MACS). Genome Biol, 2008. 9(9): p. R137.
85. Kent, W. J., et al., The human genome browser at UCSC. Genome Res, 2002. 12(6): p. 9961006.
86. Heinz, S., et al., Simple combinations of lineage-determining transcription factors prime cisregulatory elements required for macrophage and B cell identities. Mol Cell, 2010. 38(4): p. 576-89.
87. Hubbard, T., et al., The Ensembl genome database project. Nucleic Acids Res, 2002. 30(1): p. 38-41.
88. Krueger, F. and S. R. Andrews, Bismark: a flexible aligner and methylation caller for BisulfiteSeq applications. Bioinformatics, 2011. 27(11): p. 1571-2.
89. Rajala, K., et al., Formulations and methods for culturing stem cells, US20100081200A1, Published on 2010 Apr. 1

The foregoing description of the specific embodiments will so fully reveal the general nature of the disclosure that others can, by applying knowledge within the skill of the relevant art(s) (including the contents of the documents cited and incorporated by reference herein), readily modify and/or adapt for various applications such specific embodiments, without undue experimentation, without departing from the general concept of the present disclosure. Such adaptations and modifications are therefore intended to be within the meaning and range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance presented herein, in combination with the knowledge of one skilled in the relevant art(s).

While various embodiments of the present disclosure have been described above, it should be understood that they have been presented by way of examples, and not limitation. It would be apparent to one skilled in the relevant art(s) that various changes in form and detail could be made therein without departing from the spirit and scope of the disclosure. Thus, the present disclosure should not be limited by any of the above-described exemplary embodiments, but should be defined only in accordance with the following claims and their equivalents.

1. A cell culture medium for porcine cells, comprising a basal medium,
An SRC inhibitor,
Vitamin C supplement,
LIF protein, and
ACTIVIN protein.

2. The medium according to claim 1, wherein the basal medium is DMEM/F-12 or DMEM.

3. The medium according to claim 1, wherein the SRC inhibitor is WH-4-023, XAV939, IWR-1, a Tankyrase inhibitor or a combination thereof.

4. The medium according to claim 1, wherein the medium further comprises N2 supplement, B27 supplement, Glutamine Penicillin-Streptomycin, NEAA, 2-mercaptoethanol, CHIR99021, FBS, or a combination thereof.

5. A method for producing a population of porcine expanded potential stem cells (EPSCs) comprising:
(i) providing a population of porcine pluripotent cells,
(ii) culturing the population in the stem cell medium according to claim 1.

6. A cell culture medium for human cells, comprising a basal medium comprising:
An SRC inhibitor;
Vitamin C supplement; and
LIF protein.

7. The medium according to claim 6, wherein the basal medium is DMEM/F-12 or DMEM.

8. The medium according to claim 6, wherein the SRC inhibitor is A-419259, XAV939, or a combination thereof.

9. The medium according to claim 6, wherein the medium further comprises N2 supplement, B27 supplement, Glutamine Penicillin-Streptomycin, NEAA, 2-mercaptoethanol, CHIR99021, or a combination thereof.

10. A method for producing a population of human expanded potential stem cells (EPSCs) comprising:
(i) providing a population of human pluripotent cells,
(ii) culturing the population in the stem cell medium according to claim 6.

11. A cell culture medium for human cells, comprising a basal medium comprising:
ITS-X 200;
Vitamin C supplement;
Bovine Albumin Fraction V;
Trace elements B;
Trace elements C;
Reduced glutathione;
Defined lipids;
SRC inhibitor;
endo-IWR-1
SRK inhibitor; and
Chiron 99021.

12. The medium according to claim 11, wherein the basal medium is DMEM/F-12 or DMEM.

13. The medium according to claim 11, wherein the SRC inhibitor is XAV939.

14. The medium according to claim 11, wherein the SRK inhibitor is A419259.

15. The medium according to claim 11, wherein the medium further comprises Neurobasal medium, Penicillin-Streptomycin-Glutamine, NEAA, Sodium Pyruvate, 2-Mercaptoethanol, N2, B27, Human Lif protein, or a combination thereof.

16. A method for producing a population of human expanded potential stem cells (EPSCs) which comprises:
(i) providing a population of human pluripotent cells,
(ii) culturing the population in the stem cell medium according to claim 11.

17. A cell culture medium for porcine cells, comprising a basal medium comprising:
ITS-X;
Vitamin C supplement;
Bovine Albumin Fraction V;
Trace elements B;
Trace elements C;
reduced glutathione;
SRC inhibitor;
endo-IWR-1;
Chiron 99021;
Human Lif protein; and
Activin A.

18. The medium according to claim 17, wherein the basal medium is DMEM/F-12 or DMEM.

19. The medium according to claim 17, wherein the SRC inhibitor is XAV939, WH-4-023, or a combination thereof.

20. The medium according to claim 17, wherein the medium further comprises Neurobasal medium, Penicillin-Streptomycin-Glutamine, NEAA, Sodium Pyruvate, 2-Mercaptoethanol, N2, B27, or a combination thereof.

21. A method for producing a population of porcine expanded potential stem cells (EPSCs) which comprises:
(i) providing a population of porcine pluripotent cells,
(ii) culturing the population in the stem cell medium according to claim 17.

22. A porcine EPSC media, comprising:
DMEM/F-12 (Gibco, Cat. No. 21331-020), or knockout DMEM (Gibco, Cat. No. 10829-018), basal media, 98%.
N2 supplement (Thermo Fisher Scientific, Cat. No. 17502048), range from 0.1 to 1%, preferably between 0.25 to 0.75%, even preferably between 0.4-0.6%.
B27 supplement (Thermo Fisher Scientific, Cat. No. 17504044), range from 0.1 to 2%, preferably between 0.5 to 1.5%, even preferably between 0.8-1.0%.
Glutamine Penicillin-Streptomycin (Thermo Fisher Scientific, Cat. No. 11140-050), basal supplement, 1%.
NEAA (Thermo Fisher Scientific, Cat. No. 10378-016), basal supplement, 1%
2-mercaptoethanol (Sigma, Cat. No. M6250), basal supplement, 110 μM.
CHIR99021 (GSK3i, TOCRIS, Cat. No. 4423), range from 0.05 to 0.5 μM, preferably between 0.1 to 0.5 μM, even preferably between 0.2 to 0.3 μM;
WH-4-023 (SRC inhibitor, TOCRIS, Cat. No. 5413), range from 0.1 to 1.0 μM, preferably between 0.2 to 0.8 μM, even preferably between 0.3 to 0.5 μM;
XAV939 (Sigma, Cat. No. X3004), range from 1 to 10 μM, preferably between 2 to 5 μM, even preferably between 2.5 to 4.5 μM; or IWR-1 (TOCRIS, Cat. No. 3532), range from 1 to 10 μM, preferably between 2 to 5 μM, even preferably between 2.5 to 4.5 μM;
Vitamin C (Sigma, Cat. No. 49752-100G), range from 10 to 100 μg/ml, preferably between 20 to 80 μg/ml, even preferably between 50 to 70 μg/ml.
LIF (Stem Cell Institute, University of Cambridge. SCI), range from 1 to 20 ng/ml, preferably between 5 to 15 ng/ml, even preferably between 8 to 12 ng/ml.
ACTIVIN (SCI), range from 10 to 50 ng/ml, preferably between 15 to 30 ng/ml, even preferably between 20 to 25 ng/ml.
FBS (Gibco, Cat. No. 10270), range from 0.1 to 0.5%, preferably between 0.2 to 0.4%, even preferably between 0.25-0.35% and
ITS-X (thermos, 51500056), range from 0.1 to 2%, preferably between 0.2 to 0.8%, even preferably between 0.4-0.6%.

23. A human EPSC media, comprising:
DMEM/F-12 (Gibco, Cat. No. 21331-020), or knockout DMEM (Gibco, Cat. No. 10829-018), basal media, 98%.
N2 supplement (Thermo Fisher Scientific, Cat. No. 17502048),), range from 0.1 to 1%, preferably between 0.25 to 0.75%, even preferably between 0.4-0.6%.
B27 supplement (Thermo Fisher Scientific, Cat. No. 17504044), range from 0.1 to 2%, preferably between 0.5 to 1.5%, even preferably between 0.8-1.0%
Glutamine Penicillin-Streptomycin (Thermo Fisher Scientific, Cat. No. 11140-050), basal supplement, 1%
NEAA (Thermo Fisher Scientific, Cat. No. 10378-016), basal supplement, 1%
2-mercaptoethanol (Sigma, Cat. No. M6250), basal supplement, 110 μM
CHIR99021(GSK3 inhibitor, TOCRIS, Cat. No. 4423), range from 0.2 to 2 μM, preferably between 0.5 to 1.5 μM, even preferably between 0.8 to 1.2 μM.
A-419259 (SRC inhibitor, TOCRIS, Cat. No. 3914), range from 0.05 to 0.5 μM, preferably between 0.1 to 0.5 μM, even preferably between 0.15 to 0.3 μM
XAV939 (Sigma, Cat. No. X3004) range from 1 to 10 μM, preferably between 2 to 5 μM, even preferably between 2.5 to 4.5 μM or IWR-1 (TOCRIS, Cat. No. 3532), range from 1 to 10 μM, preferably between 2 to 5 μM, even preferably between 2.5 to 4.5 μM;
Vitamin C (Sigma, Cat. No. 49752-100G), range from 10 to 100 μg/ml, preferably between 20 to 80 μg/ml, even preferably between 50 to 70 μg/ml.
LIF (SCI), range from 1 to 20 ng/ml, preferably between 5 to 15 ng/ml, even preferably between 8 to 12 ng/ml

24. A human EPSC media, comprising:
DMEM/F-12 (Gibco, 21331-020), 48%
Neurobasal medium (Life Technologies, 21103-049), basal media, 48%
Penicillin-Streptomycin-Glutamine (Gibco, 10378016), basal supplement, 1%
NEAA (Gibco, 11140050), 1%
Sodium Pyruvate (gibco, 11360070), 1%
2-Mercaptoethanol (M6250 Aldrich, Sigma), basal supplement, 110 μM
N2 (Thermo 17502048), range from 0.1 to 1%, preferably between 0.25 to 0.75%, even preferably between 0.4-0.6%
B27 (Thermo 17504044), range from 0.1 to 2%, preferably between 0.5 to 1.5%, even preferably between 0.8-1.0%
ITS-X (thermos, 51500056), range from 0.1 to 1%, preferably between 0.25 to 0.75%, even preferably between 0.4-0.6%
Vitamin C (Sigma, 49752-100G), range from 10 to 100 μg/ml, preferably between 20 to 100 μg/ml, even preferably between 50 to 70 μg/ml
Bovine Albumin Fraction V (7.5% solution) (Thermo, 15260037), range from 0.1% to 1%, preferably between 0.2 to 0.8%, even preferably between 0.4-0.6%
trace elements B (Corning, MT99175CI) basal supplement, 0.1%
trace elements C (Corning, MT99176CI) basal supplement, 0.1%
reduced glutathione (sigma, G6013-5G) range from 1 to 20 μg/ml, preferably between 1 to 10 μg/ml, even preferably between 2 to 5 μg/ml
defined lipids (Invitrogen, 11905031) basal supplement, 0.2%
XAV939 (Sigma X3004), range from 1 to 10 μM, preferably between 2 to 5 μM, even preferably between 2.5 to 4.5 μM
endo-IWR-1(Tocris, Cat. No. 3532), range from 1 to 10 μM, preferably between 2 to 5 μM, even preferably between 2.5 to 4.5 μM
A419259 (Tocris Bioscience, 3748), range from 0.05 to 0.5 μM, preferably between 0.1 to 0.5 μM, even preferably between 0.15 to 0.3 μM
Chiron 99021 (Tocris Bioscience, 4423), range from 0.2 to 2 μM, preferably between 0.5 to 1.5 μM, even preferably between 0.8 to 1.2 μM and
Human Lif. (Stem Cell Institute, University of Cambridge. SCI), range from 1 to 20 ng/ml, preferably between 5 to 15 ng/ml, even preferably between 8 to 12 ng/ml

25. A porcine EPSC media, comprising:
DMEM/F-12 (Gibco, 21331-020), 48%
Neurobasal medium (Life Technologies, 21103-049), 48%
Penicillin-Streptomycin-Glutamine (Gibco, 10378016), 1%
NEAA (Gibco, 11140050), 1%
Sodium Pyruvate (gibco, 11360070), 1%
2-Mercaptoethanol (M6250 Aldrich, Sigma), basal supplement, 110 μM
N2 (Thermo 17502048), range from 0.1 to 1%, preferably between 0.25 to 0.75%, even preferably between 0.4-0.6%
B27 (Thermo 17504044), range from 0.1 to 2%, preferably between 0.5 to 1.5%, even preferably between 0.8-1.0%
ITS-X (thermos, 51500056), range from 0.1 to 1%, preferably between 0.25 to 0.75%, even preferably between 0.4-0.6%
Vitamin C (Sigma, 49752-100G), range from 10 to 100 μg/ml, between 20 to 100 μg/ml, between 50 to 70 μg/ml
Bovine Albumin Fraction V (Thermo, 15260037), range from 0.1% to 1%, between 0.2 to 0.8%, between 0.4-0.6%
trace elements B (Corning, MT99175CI) basal supplement, 0.1%
trace elements C (Corning, MT99176CI) basal supplement, 0.1%
reduced glutathione (sigma, G6013-5G) range from 1 to 20 μg/ml, preferably between 1 to 10 μg/ml, even preferably between 2 to 5 μg/ml
XAV939 (Sigma X3004), range from 1 to 10 μM, preferably between 2 to 5 μM, even preferably between 2.5 to 4.5 μM
endo-IWR-1 (Tocris, Cat. No. 3532), range from 1 to 10 μM, preferably between 1 to 5 μM, even preferably between 1 to 2 μM
WH-4-023 (Tocris, Cat. No. 5413), range from 0.1 to 1.0 μM, between 0.1 to 0.5) μM, between 0.1 to 0.2 μM
Chiron 99021 (Tocris Bioscience, 4423), range from 0.05 to 0.5 μM, preferably between 0.1 to 0.5 μM, even preferably between 0.2 to 0.3 μM
Human Lif (Stem Cell Institute, University of Cambridge. SCI), range from 1 to 20 ng/ml, preferably between 5 to 15 ng/ml, even preferably between 8 to 12 ng/ml, and
Activin A (STEM CELL TECHNOLOGY, Catalog #78001.1) range from 10 to 50 ng/ml, between 15 to 30 ng/ml, between 20 to 25 ng/ml

26. A 500 ml porcine EPSC media, comprising:
482.5 ml DMEM/F-12 (Gibco, Cat. No. 21331-020),
2.5 ml N2 supplement (Thermo Fisher Scientific, Cat. No. 17502048),
5 ml B27 supplement (Thermo Fisher Scientific, Cat. No. 17504044),
5 ml 1× Glutamine Penicillin-Streptomycin (Thermo Fisher Scientific, Cat. No. 11140-050),
5 ml 1×NEAA (Thermo Fisher Scientific, Cat. No. 10378-016),
110 μM 2-mercaptoethanol (Sigma, Cat. No. M6250),
0.2 μM CHIR99021(GSK3i, TOCRIS, Cat. No. 4423),
0.3 μM WH-4-023 (SRC inhibitor, TOCRIS, Cat. No. 5413),
2.5 μM XAV939 (Sigma, Cat. No. X3004) or 2.0 μM IWR-1 (TOCRIS, Cat. No. 3532),
50 μg/ml Vitamin C (Sigma, Cat. No. 49752-100 G),
10 ng/ml LIF (Stem Cell Institute, University of Cambridge. SCI),
20 ng/ml ACTIVIN (SCI),
1 ml ITS-X 200× (thermos, 51500056) and
0.3% FBS (Gibco, Cat. No. 10270).

27. A 500 ml human EPSC media, comprising:
482.5 ml DMEM/F-12 (Gibco, Cat. No. 21331-020),
2.5 ml N2 supplement (Thermo Fisher Scientific, Cat. No. 17502048),
5 ml B27 supplement (Thermo Fisher Scientific, Cat. No. 17504044),
5 ml 1× Glutamine Penicillin-Streptomycin (Thermo Fisher Scientific, Cat. No. 11140-050),
5 ml 1×NEAA (Thermo Fisher Scientific, Cat. No. 10378-016),
110 μM 2-mercaptoethanol (Sigma, Cat. No. M6250),
1.0 μM CHIR99021(GSK3 inhibitor, TOCRIS, Cat. No. 4423),
0.1 μM A-419259 (SRC inhibitor, TOCRIS, Cat. No. 3914),
2.5 μM XAV939 (Sigma, Cat. No. X3004) or 2.5 μM IWR-1 (TOCRIS, Cat. No. 3532),
50 μg/ml Vitamin C (Sigma, Cat. No. 49752-100 G), and
10 ng/ml LIF (SCI).

28. A 500 ml human EPSC media, comprising:
240 ml F12 DMEM (Gibco, 21331-020),
240 ml Neurobasal medium (Life Technologies, 21103-049),
5 ml Penicillin-Streptomycin-Glutamine (100×) (Gibco, 10378016),
5 ml NEAA 100× (Gibco, 11140050),
5 ml Sodium Pyruvate100× (gibco, 11360070),
110 μM 2-Mercaptoethanol (M6250 Aldrich, Sigma),
2.5 ml 200×N2 (Thermo 17502048),
5 ml 100×B27 (Thermo 17504044),
2.5 ml ITS-X 200× (thermos, 51500056),
64 ug/ml Vitamin C (Sigma, 49752-100G),
3 ml Bovine Albumin Fraction V (7.5% solution) (Thermo, 15260037),
Trace elements B, (Corning, MT99175CI) 1000×
Trace elements C, (Corning, MT99176CI) 1000×
165 ul reduced glutathione (sigma, G6013-5G) 10 mg/ml,
defined lipids, (Invitrogen, 11905031) 500×
2.5 μM XAV939 (Sigma X3004),
2.5 μM endo-IWR-1(Tocris, Cat. No. 3532),
0.1 μM A419259 (Tocris Bioscience, 3748),
1.0 μM Chiron 99021 (Tocris Bioscience, 4423), and
10 ng/ml Human Lif.

29. A 500 ml porcine EPSC media, comprising:
240 ml F12 DMEM (Gibco, 21331-020),
240 ml Neurobasal medium (Life Technologies, 21103-049),
5 ml Penicillin-Streptomycin-Glutamine (100×) (Gibco, 10378016),
5 ml NEAA 100× (Gibco, 11140050),
5 ml Sodium Pyruvate100× (gibco, 11360070),
110 μM 2-Mercaptoethanol (M6250 Aldrich, Sigma),
2.5 ml 200×N2 (Thermo 17502048),
5 ml 100×B27 (Thermo 17504044),
2.5 ml ITS-X 200×(thermos, 51500056),
64 ug/ml Vitamin C (Sigma, 49752-100G),
3 ml Bovine Albumin Fraction V (7.5% solution) (Thermo, 15260037),
Trace elements B, (Corning, MT99175CI) 1000×
race elements C, (Corning, MT99176CI) 1000×
165 ul reduced glutathione (sigma, G6013-5G) 10 mg/ml,
2.5 μM XAV939 (Sigma X3004),
1 μM endo-IWR-1(Tocris, Cat. No. 3532),
0.16 μM WH-4-023 (Tocris, Cat. No. 5413),
0.2 μM Chiron 99021 (Tocris Bioscience, 4423),
10 ng/ml Human Lif, and
20 ng/ml Activin A (STEM CELL TECHNOLOGY, Catalog #78001.1).
